(19) United States
(12) Patent Application Publication
(10) Pub. No.: US 2010/0056502 A1
(43) Pub. Date: Mar. 4, 2010

(54) COMPOUNDS

(75) Inventors: Pamela Brown, Hertfordshire (GB); Steven Dabbs, Hertfordshire (GB); David Thomas Davies, Hertfordshire (GB); Neil David Pearson, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 12/532,639

(22) PCT Filed: Mar. 20, 2008
(86) PCT No.: PCT/EP08/53350

§ 371 Date: Sep. 23, 2009

(30) Foreign Application Priority Data
Mar. 23, 2007 (GB) 0705672.4

(51) Int. Cl. A61K 31/542; C07D 513/00; C07D 498/04; A61K 31/5365; C07D 487/00; A61K 31/5025; C07D 453/00; A61K 31/4375

(52) U.S. Cl. 514/224.2; 544/48; 544/101; 514/229.8; 544/236; 514/248; 546/94; 514/294

(57) ABSTRACT

Tricyclic nitrogen containing compounds and their use as antibacterials.

This invention relates to novel compounds, compositions containing them and their use as antibacterials including the treatment of tuberculosis.

WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO06002047, WO06014580, WO06010040, WO06017326, WO06012396, WO06017468, WO06020561, WO06081179, WO06081264, WO06081289, WO06081178, WO06081182, WO01/25227, WO02/40474, WO02/07572, WO2004035569, WO04024712, WO04024713, WO04087647, WO2005016916, WO2005097781, WO06010831, WO04089947, WO06021448, WO06032466, WO06038172, WO06046552, WO06099884, WO06105289, WO06126171, WO06125974, WO06134378, WO06137485, WO07016610, WO07081597, WO07071936, WO07115947, WO07118130, WO07122258, WO08006648, WO08003690 and WO08009700 disclose quinoline, naphthyridine, morpholine, cyclohexane, piperidine and piperazine derivatives having antibacterial activity.

This invention provides a compound of formula (I) or a pharmaceutically acceptable salt and/or N-oxide thereof:

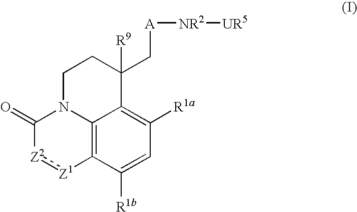

wherein:
Z¹ and Z² together are CH═CH or S or Z¹ is O and Z² is CH₂;
R^{1a} and R^{1b} are independently selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; or aminocarbonyl wherein the amino group is optionally substituted by (C₁₋₄)alkyl;
R² is hydrogen, or (C₁₋₄)alkyl, or together with R⁶ forms Y as defined below;
A is a group (i):

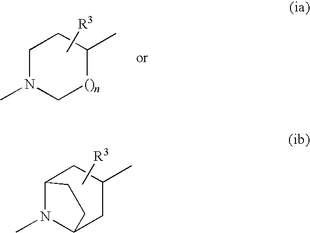

in which: R³ is as defined for R^{1a} or R^{1b} or is oxo and n is 1 or 2:
or A is a group (ii)

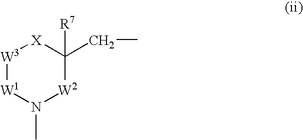

W¹, W² and W³ are CR⁴R⁸;

or W² and W³ are CR⁴R⁸ and W¹ represents a bond between W³ and N;

X is O, CR⁴R⁸, or NR⁶;

one R⁴ is as defined for R^{1a} and R^{1b} and the remainder and R⁸ are hydrogen or one R⁴ and R⁸ are together oxo and the remainder are hydrogen;

R⁶ is hydrogen or (C₁₋₆)alkyl; or together with R² forms Y;

R⁷ is hydrogen; halogen; hydroxy optionally substituted with (C₁₋₆)alkyl; or (C₁₋₆)alkyl;

Y is CR⁴R⁸CH₂; CH₂CR⁴R⁸; (C═O); CR⁴R⁸; CR⁴R⁸(C═O); or (C═O)CR⁴R⁸;

or when X is CR⁴R⁸, R⁸ and R⁷ together represent a bond;

U is selected from CO and CH₂ and
R⁵ is an optionally substituted bicyclic carbocyclic or heterocyclic ring system (B):

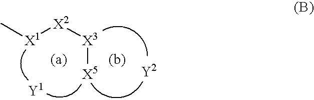

containing up to four heteroatoms in each ring in which

at least one of rings (a) and (b) is aromatic;

X¹ is C or N when part of an aromatic ring, or CR¹⁴ when part of a non-aromatic ring;

X² is N, NR¹³, O, S(O)ₓ, CO or CR¹⁴ when part of an aromatic or non-aromatic ring or may in addition be CR¹⁴R¹⁵ when part of a non aromatic ring;

X³ and X⁵ are independently N or C;

Y¹ is a 0 to 4 atom linker group each atom of which is independently selected from N, NR¹³, O, S(O)_{X}, CO and CR¹⁴ when part of an aromatic or non-aromatic ring or may additionally be CR¹⁴R¹⁵ when part of a non aromatic ring;

Y² is a 2 to 6 atom linker group, each atom of Y² being independently selected from N, NR¹³, O, S(O)_{X}, CO, CR¹⁴ when part of an aromatic or non-aromatic ring or may additionally be CR¹⁴R¹⁵ when part of a non aromatic ring;

each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or

R¹⁴ and R¹⁵ may together represent oxo;

each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; and

each x is independently 0, 1 or 2; and

R⁹ is hydrogen or hydroxy.

In one aspect Z¹ and Z² together are CH═CH or S.

This invention also provides a method of treatment of bacterial infections including tuberculosis in mammals, particularly in man, which method comprises the administration to a mammal in need of such treatment an effective amount of a compound of formula (I), or a pharmaceutically acceptable salt and/or N-oxide thereof.

The invention also provides the use of a compound of formula (I), or a pharmaceutically acceptable salt and/or N-oxide thereof, in the manufacture of a medicament for use in the treatment of bacterial infections including tuberculosis in mammals.

The invention also provides a pharmaceutical composition comprising a compound of formula (I), or a pharmaceutically acceptable salt and/or N-oxide thereof, and a pharmaceutically acceptable carrier.

In a particular aspect each R^{1a} and R^{1b} is independently hydrogen, (C₁₋₄)alkoxy, (C₁₋₄)alkylthio, (C₁₋₄)alkyl, cyano, carboxy, hydroxymethyl or halogen; more particularly hydrogen, methoxy, methyl, cyano, or halogen.

In certain embodiments only one group R^{1a} and R^{1b} is other than hydrogen. In particular embodiments Z¹ and Z² together are CH═CH, R^{1a} is fluoro or methoxy and R^{1b} is hydrogen, or Z¹ and Z² together are S, R^{1a} is fluoro or chloro and R^{1b} is hydrogen. In other embodiments Z¹ and Z² together represent CH₂O, R^{1a} is fluoro and R^{1b} is hydrogen.

In other embodiments R^{1a} and R^{1b} are both hydrogen.

In a particular aspect R² is hydrogen.

Particular examples of R³ include hydrogen; optionally substituted hydroxy; optionally substituted amino; halogen; (C₁₋₄) alkyl; 1-hydroxy-(C₁₋₄) alkyl; optionally substituted aminocarbonyl. More particular R³ groups are hydrogen; CONH₂; 1-hydroxyalkyl e.g. CH₂OH; optionally substituted hydroxy e.g. methoxy; optionally substituted amino; and halogen, in particular fluoro. Most particularly R³ is hydrogen, hydroxy or fluoro.

In a particular aspect, when A is (ia), n is 1. In a further aspect R³ is in the 3- or 4-position. In a more particular aspect, A is (ia), n is 1 and R³ is in the 3-position, and more particularly is cis to the NR² group.

In particular embodiments, A is a group (ia) in which n is 1 and R³ is hydrogen or hydroxy.

More particularly where A is 3-hydroxy-piperidin-4-yl the configuration is (3R,4S) or (3S,4R).

Alternatively and more particularly where A is piperidin-4-yl the configuration is (3R,4S).

In a particular aspect, when A is (ii), X is CR⁴R⁸, R⁸ is H and R⁴ is H or OH. More particularly when R⁴ is OH it is trans to R⁷. In a further aspect W¹ is a bond. In another aspect R⁷ is H. In an additional aspect W¹ is a bond, W² and W³ are both CH₂ and R⁷ is H. Where A is 4-hydroxypyrrolidin-3-ylmethyl, in a particular aspect the configuration is (3S,4S).

In a particular aspect, when A is (ii), X is O, R⁷ is H and W¹, W² and W³ are each CH₂.

In certain embodiments U is CH₂.

In certain embodiments R⁵ is an aromatic heterocyclic ring (B) having 8-11 ring atoms including 2-4 heteroatoms of which at least one is N or NR¹³ in which, in particular embodiments, Y² contains 2-3 heteroatoms, one of which is 5 and 1-2 are N, with one N bonded to X³.

In alternative embodiments the heterocyclic ring (B) has ring (a) aromatic selected from optionally substituted benzo, pyrido, pyridazino and pyrimidino and ring (b) non aromatic and Y² has 3-5 atoms, more particularly 4 atoms, including at least one heteroatom, with O, S, CH₂ or NR¹³ bonded to X⁵ where R¹³ is other than hydrogen, and either NHCO bonded via N to X³, or O, S, CH₂ or NH bonded to X³. In a particular aspect the ring (a) contains aromatic nitrogen, and more particularly ring (a) is pyridine or pyrazine. Examples of rings (B) include optionally substituted:

### (a) and (b) Aromatic

1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, naphthalen-2-yl, 1,3-dioxo-isoindol-2-yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-6-yl, pyrido[1,2-a]pyrimidin-4-one-3-yl (4-oxo-4H-pyrido[1,2-a]pyrimidin-3-yl), benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimdin-4-one-2-yl (4-oxo-4H-pyrido[1,2-a]pyrimidin-2-yl), quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, thiazolo[4,5-b]pyridin-5-yl, [1,2,3]thiadiazolo[5,4-b]pyridin-6-yl, 2H-isoquinolin-1-one-3-yl (1-oxo-1,2-dihydro-isoquinolin-3-yl)

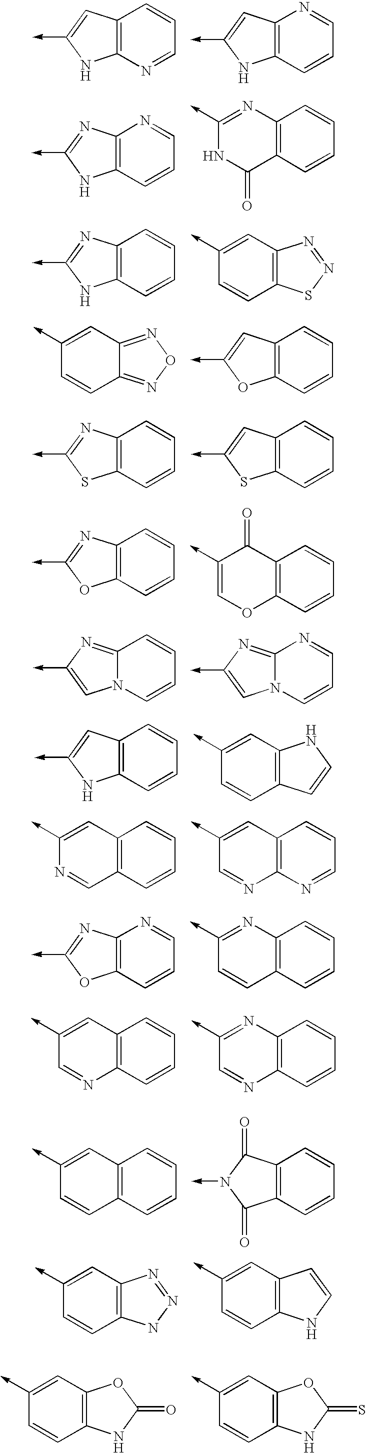

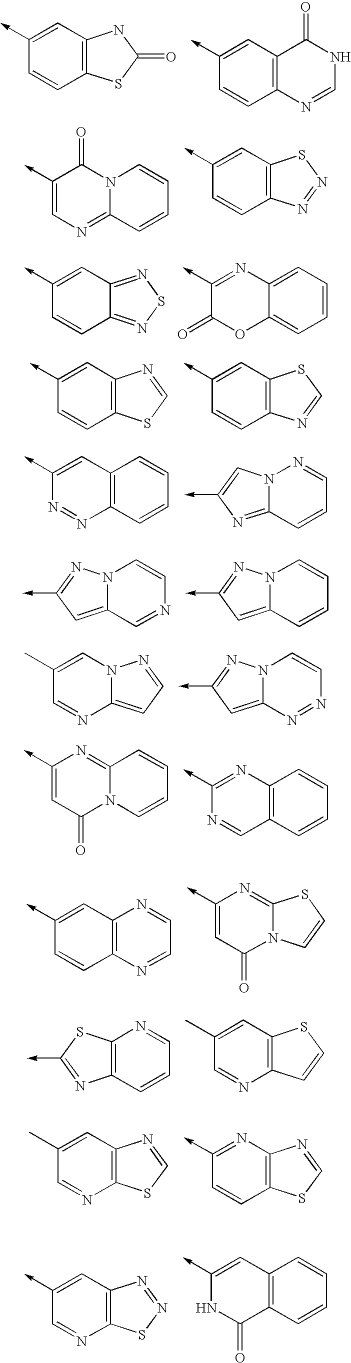

→is the point of attachment

### (a) is Non Aromatic

(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl,

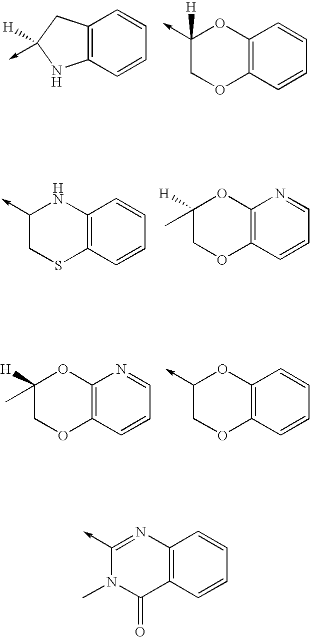

→is the point of attachment

### (b) is Non Aromatic

1,1,3-trioxo-1,2,3,4-tetrahydrol l⁶-benzo[1,4]thiazin-6-yl, benzo[1,3]dioxol-5-yl, 2,3-dihydro-benzo[1,4]dioxin-6-yl, 3-substituted-3H-benzooxazol-2-one-6-yl, 3-substituted-3H-benzooxazole-2-thione-6-yl, 3-substituted-3H-benzothiazol-2-one-6-yl, 4H-benzo[1,4]oxazin-3-one-6-yl (3-oxo-3,4-dihydro-2H-benzo[1,4]oxazin-6-yl), 4H-benzo[1,4]thiazin-3-one-6-yl (3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, 1H-pyrido[2,3-b][1,4]thiazin-2-one-7-yl (2-oxo-2,3-dihydro-1H-pyrido[2,3-b]thiazin-7-yl), 2,3-dihydro-1H-pyrido[2,3-b][1,4]thiazin-7-yl, 2-oxo-2,3-dihydro-1H-pyrido[3,4-b]thiazin-7-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 3,4-dihydro-2H-benzo[1,4]oxazin-6-yl, 3,4-dihydro-2H-benzo[1,4]thiazin-6-yl, 3-oxo-3,4-dihydro-2H-pyrido[3,2-b][1,4]oxazin-6-yl, 3,4-dihydro-2H-pyrido[3,2-b][1,4]thiazin-6-yl, 3-oxo-3,4-dihydro-2H-pyrido[3,2-b][1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl (1,2,3,4-tetrahydro-[1,8]naphthyridin-7-yl), 2-oxo-3,4-dihydro-1H-[1,8]naphthyridin-6-yl, 6-oxo-6,7-dihydro-5H-pyridazino[3,4-b][1,4]thiazin-3-yl (6-oxo-6,7-dihydro-5H-8-thia-1,2,5-triaza-naphthalen-3-yl), 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6,7-dihydro-[1,4]dioxino[2,3-d]pyrimidin-2-yl, [1,3]oxathiolo[5,4-c]pyridin-6-yl, 3,4-dihydro-2H-pyrano[2,3-c]pyridin-6-yl, 2,3-dihydro[1,4]oxathiino[2,3-c]pyridine-7-yl, 6,7-dihydro[1,4]oxathiino[2,3-c]pyridazin-3-yl, 6,7-dihydro[1,4]dioxino[2,3-c]pyridazin-3-yl, 6,7-dihydro-5H-pyrano[2,3-c]pyridazin-3-yl, 5,6-dihydrofuro[2,3-c]pyridazin-3-yl, 2,3-dihydrofuro[2,3-c]pyridin-5-yl, 2-substituted 1H-pyrimido[5,4-b][1,4]oxazin-7(6H)-one, 2-substituted 5,6-dihydropyrido[2,3-d]pyrimidin-7(1H)-one, 7-substituted 2H-chromen-2-one, 7-substituted 2H-pyrano[2,3-b]pyridin-2-one, 2-substituted 6,7-dihydro-5H-pyrano[2,3-d]pyrimidine, 8-substituted 2H-pyrido[1,2-a]pyrimidin-2-one, 2,3-dihydro-1-benzofuran-5-yl.

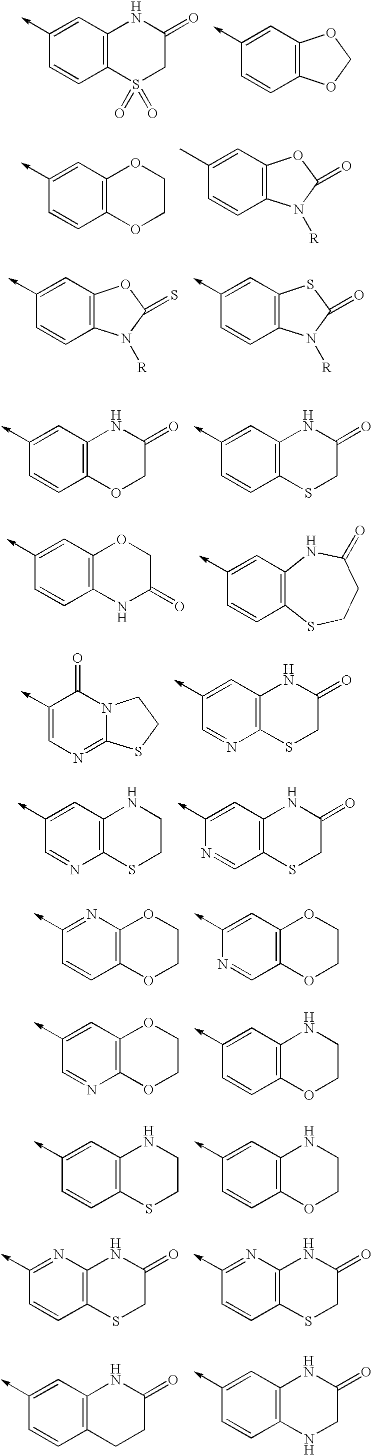

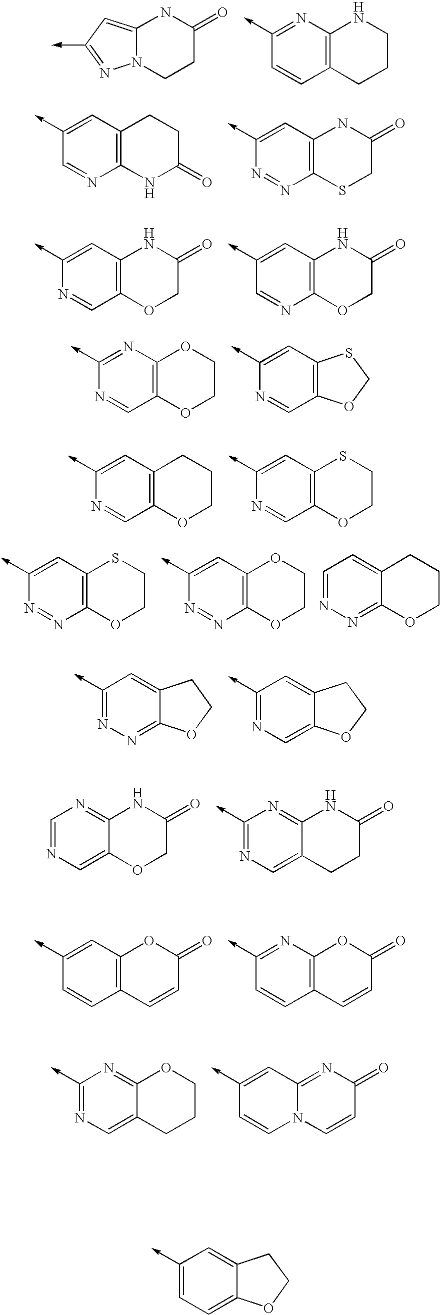

where R is an optional substituent
→is the point of attachment

In some embodiments R¹³ is H if in ring (a) or in addition (C₁₋₄)alkyl such as methyl or isopropyl when in ring (b). More particularly, in ring (b) R¹³ is H when NR¹³ is bonded to X³ and (C₁₋₄)alkyl when NR¹³ is bonded to X⁵.

In further embodiments R¹⁴ and R¹⁵ are independently selected from hydrogen, halo, hydroxy, (C₁₋₄) alkyl, (C₁₋₄)alkoxy, nitro and cyano. More particularly R¹⁵ is hydrogen.

More particularly each R¹⁴ is selected from hydrogen, chloro, fluoro, hydroxy, methyl, methoxy, nitro and cyano. Still more particularly R¹⁴ is selected from hydrogen, fluorine or nitro.

Most particularly R¹⁴ and R¹⁵ are each H.

Particular groups R⁵ include:
[1,2,3]thiadiazolo[5,4-b]pyridin-6-yl
1H-pyrrolo[2,3-b]pyridin-2-yl
2,3-dihydro-[1,4]dioxino[2,3-b]pyridin-6-yl
2,3-dihydro-[1,4]dioxino[2,3-b]pyridin-7-yl
2,3-dihydro-[1,4]dioxino[2,3-c]pyridin-7-yl
2,3-dihydro-benzo[1,4]dioxin-6-yl
2-oxo-2,3-dihydro-1H-pyrido[2,3-b][1,4]oxazin-7-yl
2-oxo-2,3-dihydro-1H-pyrido[2,3-b][1,4]thiazin-7-yl
3,4-dihydro-2H-benzo[1,4]oxazin-6-yl
3-methyl-2-oxo-2,3-dihydro-benzooxazol-6-yl
3-oxo-3,4-dihydro-2H-benzo[1,4]oxazin-6-yl
3-oxo-3,4-dihydro-2H-pyrido[3,2-b][1,4]oxazin-6-yl
3-oxo-3,4-dihydro-2H-benzo[1,4]thiazin-6-yl (4H-benzo[1,4]thiazin-3-one-6-yl)
4-oxo-4H-pyrido[1,2-a]pyrimidin-2-yl
6-nitro-benzo[1,3]dioxol-5-yl
7-fluoro-3-oxo-3,4-dihydro-2H-benzo[1,4]oxazin-6-yl
8-hydroxy-1-oxo-1,2-dihydro-isoquinolin-3-yl
8-hydroxyquinolin-2-yl
benzo[1,2,3]thiadiazol-5-yl
benzo[1,2,5]thiadiazol-5-yl
benzothiazol-5-yl
thiazolo-[5,4-b]pyridin-6-yl
3-oxo-3,4-dihydro-2H-pyrido[3,2-b][1,4]thiazin-6-yl
7-chloro-3-oxo-3,4-dihydro-2H-pyrido[3,2-b][1,4]thiazin-6-yl
7-chloro-3-oxo-3,4-dihydro-2H-pyrido[3,2-b][1,4]oxazin-6-yl
7-fluoro-3-oxo-3,4-dihydro-2H-pyrido[3,2-b][1,4]thiazin-6-yl
2-oxo-2,3-dihydro-1H-pyrido[3,4-b][1,4]thiazin-7-yl
[1,3]oxathiolo[5,4-c]pyridin-6-yl
3,4-dihydro-2H-pyrano[2,3-c]pyridin-6-yl
5-carbonitro-2,3-dihydro-1,4-benzodioxin-7-yl
2,3-dihydro[1,4]oxathiino[2,3-c]pyridin-7-yl
6,7-dihydro[1,4]oxathiino[2,3-c]pyridazin-3-yl
6,7-dihydro[1,4]dioxino[2,3-c]pyridazin-3-yl
2,3-dihydro-1-benzofuran-5-yl
6,7-dihydro-5H-pyrano[2,3-c]pyridazin-3-yl
5,6-dihydrofuro[2,3-c]pyridazin-3-yl
2-substituted 1H-pyrimido[5,4-b][1,4]oxazin-7(6H)-one
2-substituted 4-chloro-1H-pyrimido[5,4-b][1,4]oxazin-7(6H)-one
2-substituted 5,6-dihydropyrido[2,3-d]pyrimidin-7(1H)-one
2-substituted 4-chloro-5,6-dihydropyrido[2,3-d]pyrimidin-7(1H)-one
2-substituted 4-methyl-5,6-dihydropyrido[2,3-d]pyrimidin-7(1H)-one
2-substituted 4-methyloxy-5,6-dihydropyrido[2,3-d]pyrimidin-7(1H)-one
7-substituted 2H-chromen-2-one
7-substituted 2H-pyrano[2,3-b]pyridin-2-one
4-chloro-6,7-dihydro-5H-pyrano[2,3-d]pyrimidin-2-yl
8-substituted 2H-pyrido[1,2-a]pyrimidin-2-one
6,7-dihydro-5H-pyrano[2,3-d]pyrimidin-2-yl
5-chloro-1-benzothiophen-2-yl
6-chloro-1-benzothiophen-2-yl
1-benzothiophen-5-yl
1-methyl-1H-1,2,3-benzotriazol-6-yl
imidazo[2,1-b][1,3]thiazol-6-yl
4-methyl-3,4-dihydro-2H-1,4-benzoxazin-7-yl
1-methyl-1H-indol-2-yl

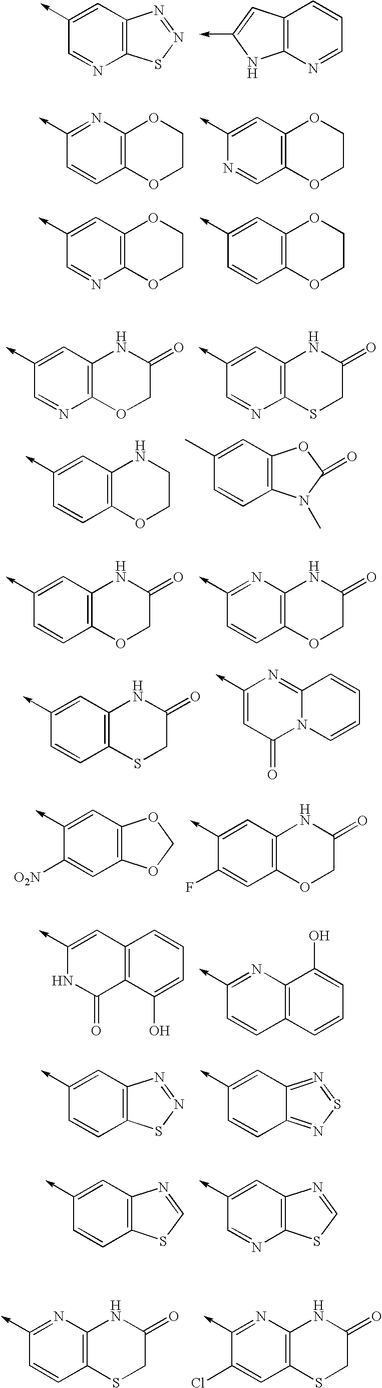

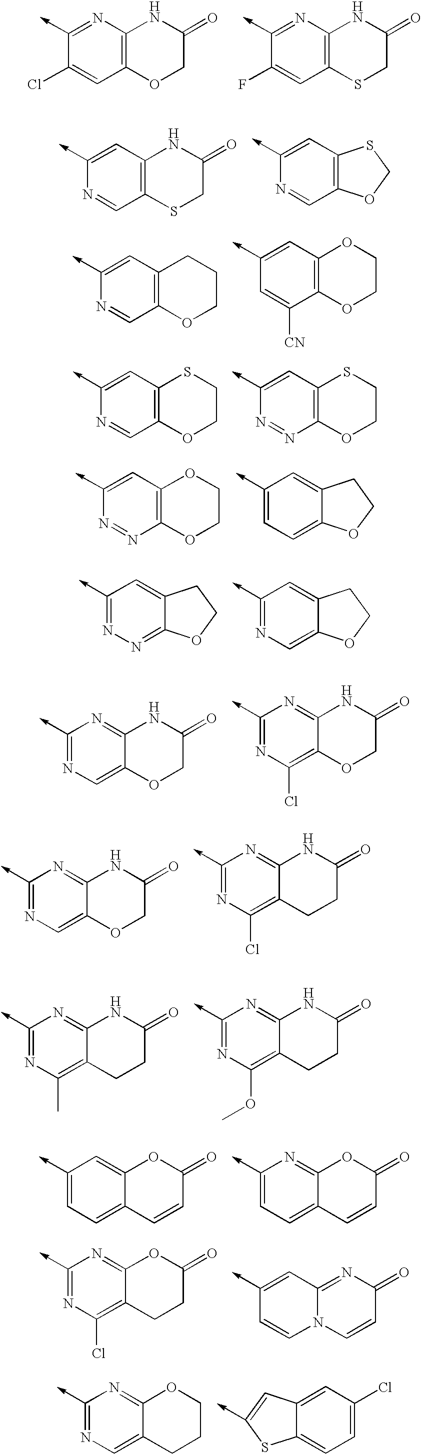

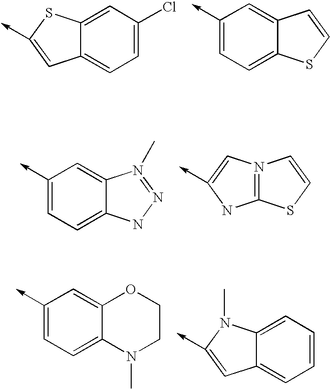

especially
3-oxo-3,4-dihydro-2H-pyrido[3,2-b][1,4]oxazin-6-yl
3-oxo-3,4-dihydro-2H-pyrido[3,2-b][1,4]thiazin-6-yl
6,7-dihydro[1,4]dioxino[2,3-c]pyridazin-3-yl
6,7-dihydro[1,4]oxathiino[2,3-c]pyridazin-3-yl
2,3-dihydro-[1,4]dioxino[2,3-c]pyridin-7-yl
[1,3]oxathiolo[5,4-c]pyridin-6-yl.

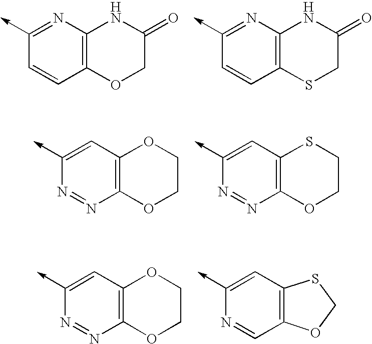

When used herein, the term “alkyl” includes groups having straight and branched chains, for instance, methyl, ethyl, n-propyl, iso-propyl, n-butyl, iso-butyl, sec-butyl, t-butyl, pentyl and hexyl. The term ‘alkenyl’ should be interpreted accordingly.

Halo or halogen includes fluoro, chloro, bromo and iodo.

Haloalkyl moieties include 1-3 halogen atoms.

Compounds within the invention contain a heterocyclyl group and may occur in two or more tautomeric forms depending on the nature of the heterocyclyl group; all such tautomeric forms are included within the scope of the invention.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Furthermore, it will be understood that phrases such as “a compound of formula (I) or a pharmaceutically acceptable salt or N-oxide thereof” are intended to encompass the compound of formula (I), an N-oxide of formula (I), a pharmaceutically acceptable salt of the compound of formula (I) or any pharmaceutically acceptable combination of these. By way of non-limiting example used here for illustrative purpose, “a compound of formula (I) or a pharmaceutically acceptable salt thereof” may include a pharmaceutically acceptable salt of a compound of formula (I) that is further present as a solvate.

Since the compounds of formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10 to 59% of a compound of the formula (I) or pharmaceutically acceptable salt and/or N-oxide thereof.

Particular compounds according to the invention include those mentioned in the examples and their pharmaceutically acceptable salts or N-oxides.

Pharmaceutically acceptable salts of the above-mentioned compounds of formula (I) include the acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. The invention extends to all such derivatives.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. For example the invention includes enantiomers and diastereoisomers at the attachment point of NR² and R³. The different isomeric forms may be separated or resolved one from the other by conventional methods, or any given isomer may be obtained by conventional synthetic methods or by stereospecific or asymmetric syntheses. Certain compounds of formula (I) may also exist in polymorphic forms and the invention includes such polymorphic forms.

In a further aspect of the invention there is provided a process for preparing compounds of formula (I) in which R⁹ is H, and pharmaceutically acceptable salt and/or N-oxide thereof, which process comprises reacting a compound of formula (IIA) with a compound of formula (III):

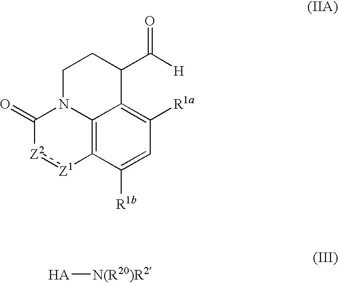

in which:
R²⁰ is UR⁵ or a group convertible thereto and R^{2′} is R² or a group convertible thereto, wherein Z¹ and Z², A, R^{1a}, R^{1b}, R², U and R⁵ are as defined in formula (I), and thereafter optionally or as necessary converting R²⁰ and R^{2′} to UR⁵ and R², interconverting any variable groups, and/or forming a pharmaceutically acceptable salt or N-oxide thereof.

The reaction is a reductive alkylation (see for examples Smith, M. B.; March, J. M. Advanced Organic Chemistry, Wiley-Interscience 2001) with a suitable reducing agent such as sodium cyanoborohydride (in methanol/chloroform/acetic acid), triacetoxyborohydride or (polystyrylmethyl)trimethylammonium cyanoborohydride. If the amine is present as a hydrochloride salt it is preferable to have an excess of sodium acetate present to buffer the reaction. 3A Molecular sieves may also be used to help formation of the initial imine intermediate. The compound of formula (IIA) may be presented as a hemiacetal.

In a further aspect of the invention there is provided a process for preparing compounds of formula (I) in which R⁹ is OH, and pharmaceutically acceptable salt and/or N-oxide thereof, which process comprises reacting a compound of formula (IIB) with a compound of formula (III):

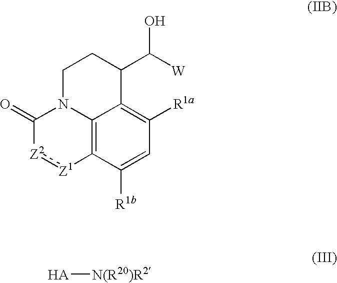

W is a leaving group, R²⁰ is UR⁵ or a group convertible thereto and R^{2′} is R² or a group convertible thereto, wherein Z¹ and Z², A, R^{1a}, R^{1b}, R², U and R⁵ are as defined in formula (I), and thereafter optionally or as necessary converting R²⁰ and R^{2′} to UR⁵ and R², interconverting any variable groups, and/or forming a pharmaceutically acceptable salt or N-oxide thereof.

The leaving group W may be any conventional group such as methanesulfonyl or methylbenzenesulfonyl. The reaction is carried out under conventional conditions for amine coupling such as reacting together in the presence of a suitable base, such as sodium carbonate or triethylamine, in a suitable solvent such as ethanol or N,N-dimethylformamide at temperatures between ambient and 60° C. Where R⁹ is OH, treatment with base can afford an epoxide which can react with amine (III). Such reactions may proceed through this epoxide without the need for isolation.

The reaction of (IIA) or (IIB) and (III) gives a compound of formula (IIC):

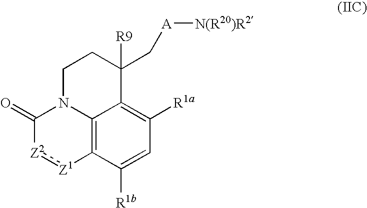

The invention further provides compounds of formula (IIC) in which R²⁰ is hydrogen.

In the reactions above, conveniently one of R²⁰ and R^{2′} is an N-protecting group, such as such as t-butoxycarbonyl, benzyloxycarbonyl, 9-fluorenylmethyloxycarbonyl or trifluoroacetyl. This may be removed by several methods well known to those skilled in the art (for examples see “Protective Groups in Organic Synthesis, T. W. Greene and P. G. M. Wuts, Wiley-Interscience, 1999), for example conventional acid hydrolysis (e.g. trifluoroacetic acid/dichloromethane, hydrochloric acid/dichloromethane/methanol), or potassium carbonate/methanol.

The free amine of formula (IIC) in which R²⁰ is hydrogen may be converted to NR²UR⁵ by conventional means such as amide formation with an acyl derivative R⁵COW, for compounds where U is CO or, where U is CH₂, by alkylation with an alkyl halide R⁵CH₂-halide in the presence of base, acylation/reduction with an acyl derivative R⁵COW or reductive alkylation with an aldehyde R⁵CHO under conventional conditions (see for examples Smith, M. B.; March, J. M. Advanced Organic Chemistry, Wiley-Interscience 2001). Suitable conditions include sodium cyanoborohydride (in methanol/chloroform/acetic acid) or (polystyrylmethyl)trimethylammonium cyanoborohydride. If the amine (III) is a hydrochloride salt then sodium acetate may be added to buffer the reaction. Sodium triacetoxyborohydride is an alternative reducing agent.

The appropriate reagents containing the required R⁵ group are known compounds or may be prepared analogously to known compounds, see for example WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2004/035569, WO2004/089947, WO2003082835, WO06002047, WO06014580, WO06010040, WO06017326, WO06012396, WO06017468, WO06020561, WO06132739, WO06134378, WO06137485, WO06081179, WO06081264, WO06081289, WO06081178, WO06081182, WO07016610, WO07081597, WO07071936, WO07115947, WO07118130, WO07122258, WO08006648, WO08003690 and WO08009700 and EP0559285.

Where R⁵ contains an NH group, this may be protected with a suitable N-protecting group such as t-butoxycarbonyl, benzyloxycarbonyl or 9-fluorenylmethyloxycarbonyl during the coupling of the R⁵ derivative with the free amine of formula (IIB). The protecting group may be removed by conventional methods, such as by treatment with trifluoroacetic acid.

Compounds of formula (IIA) may be prepared by the following Scheme 1:

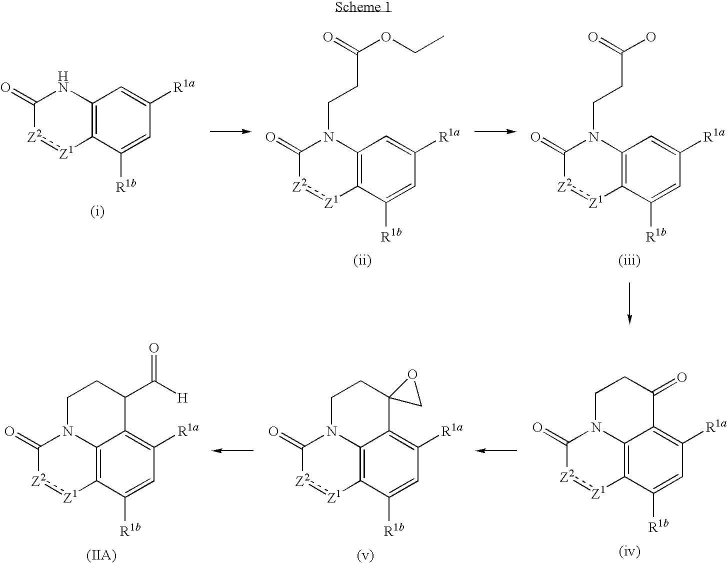

Compounds of structure (ii) may be made by alkylation of compounds of type (i), under conventional alkylation conditions on treatment with a suitable electrophile in the presence of base, optionally under phase-transfer conditions (see for example G. McCort et al, Bioorg Med Chem, 2001, 2129,) or utilising conjugate addition of the amide to ethyl acrylate (for example as described by K. H. Ahn, and S. J. Lee, Tetrahedron Letters 1994, 35, 1835). Compounds (ii) can be converted to compounds (iii) by hydrolysis under conventional conditions, or alternatively, compounds (i) can be converted directly to compounds (iii) by reaction with an appropriate electrophile under the general N-alkylation conditions mentioned herein. Compounds of type (iii) can be converted into compounds of type (iv) by conventional methods of Friedel-Crafts acylation. Examples of this method are treatment of compound (iii) with polyphosphoric acid at temperatures from room temperature to 120° C., or by activation of compound (iii) to the acid chloride using oxalyl chloride in the presence of catalytic DMF, followed by treatment of the acid chloride with a conventional Lewis-acid, for example aluminium trichloride (see for example Smith, M. B.; March, J. M. Advanced Organic Chemistry, Wiley-Interscience 2001). Compounds of type (iv) can be converted into compounds of type (v) on treatment with dimethylsulfoxonium methylide according to standard methods (for example see A. W, Beck et al, J. Chem Soc Perkin 1, 1990, 689). Conversion of epoxides to aldehydes by mild Lewis Acid catalysis is a transformation well known in the literature (see for example J. G. Smith, Synthesis, 1984, 629) and the epoxide (v) can be converted into compound (IIA) on treatment with acidic aluminium oxide.

Alternatively the compound of formula (IIA) may be prepared from (iv) via (vi), see Scheme 2:

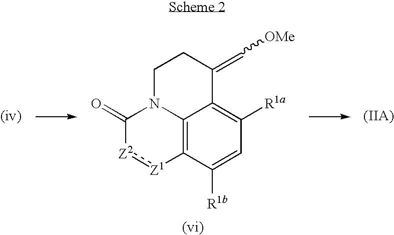

Compounds (iv) may be converted via conventional Wittig methoxymethylenation to (vi) (for an example see D. Boger et al, J. Org Chem 1990, 1919) followed by acid-catalysed hydrolysis, using either mineral or organic acid (e.g. formic acid) or alternatively using chlorotrimethyl silane/sodium iodide (using the method of I Ernest et al, Helv. Chim Acta, 1993, 1539).

Compounds of formula (IIB) may be prepared by the following Scheme 3:

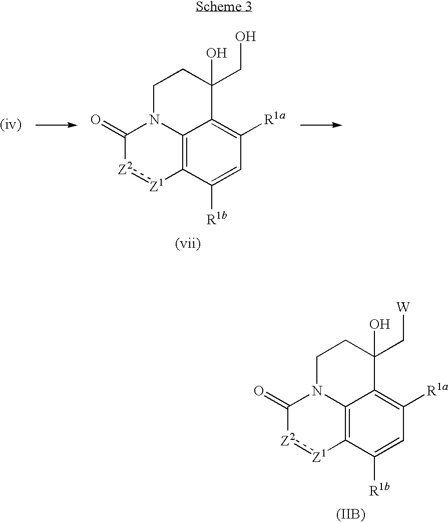

When compounds of type (iv) (Scheme 1) are treated with dimethylsulfoxonium methylide under standard conditions followed by aqueous or acidic work-up, diols of type (vii) can be isolated. The primary alcohol of diols (vii) can be selectively activated towards displacement by reaction with sulphonyl halides (see Wallner, Sabine R, Organic & Biomolecular Chemistry (2005) 3(14), 2652-2656) and this selectivity may be enhanced by the use of catalytic quantities of dibutyltin oxide (see Boger, Dale, Journal of the American Chemical Society (1996) 118(9), 2301-2).

Alternatively the compound of formula (vii) may be prepared by the following Scheme 4:

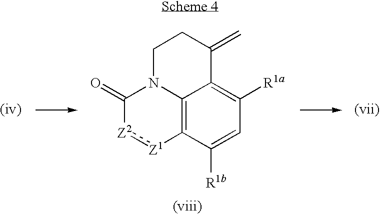

Compounds of type (iv) (Scheme 1) can be converted into compounds (viii) using conventional Wittig chemistry by reaction with methyl triphenylphosphonium bromide in the presence of base (see for example Smith, M. B.; March, J. M. Advanced Organic Chemistry, Wiley-Interscience 2001).

Oxidation of vinyl derivatives (viii) to diols (vii) may be accomplished with standard reagent systems such as osmium tetroxide/N-methyl morpholine-N-oxide

(Zheng, Tao et al, Journal of the American Chemical Society (2005) 127(19), 6946-6947). It will be appreciated that such oxidations can be performed chirally using appropriate chiral oxidising systems such as ADmix alpha or beta (see Pinard, E et al, Bioorganic & Medicinal Chemistry Letters (2001), 11(16), 2173-2176

Interconversions of Z¹, Z², R^{1a}, R^{1b}, R², A and R⁵ are conventional. In compounds which contain an optionally protected hydroxy group, suitable conventional hydroxy protecting groups which may be removed without disrupting the remainder of the molecule include acyl and alkylsilyl groups. N-protecting groups are removed by conventional methods.

Interconversion of R^{1a} and R^{1b} groups may be carried out conventionally, on compounds of formula (I), (IIA), (IIB) or (IIC). For example R^{1a} or R^{1b} methoxy is convertible to R^{1a} or R^{1b} hydroxy by treatment with lithium and diphenylphosphine (general method described in Ireland et al, J. Amer. Chem. Soc., 1973, 7829) or HBr. Alkylation of the hydroxy group with a suitable alkyl derivative bearing a leaving group such as halide, yields R^{1a} or R^{1b} substituted alkoxy. R^{1a} halogen is convertible to other R^{1a} by conventional means, for example to hydroxy, alkylthiol (via thiol) and amino using metal catalysed coupling reactions, for example using copper as reviewed in Synlett (2003), 15, 2428-2439 and Angewandte Chemie, International Edition, 2003, 42(44), 5400-5449. R^{1a} fluoro may be converted to methoxy by treatment with sodium methoxide in a suitable solvent such as methanol and optionally dichloromethane. R^{1a} or R^{1b} halo such as bromo may be converted to cyano by treatment with copper (I) cyanide in N,N-dimethylformamide. R^{1a} or R^{1b} carboxy may be obtained by conventional hydrolysis of R^{1a} or R^{1b} cyano, and the carboxy converted to hydroxymethyl by conventional reduction.

Compounds of formula (i) are known compounds or may be prepared analogously to known compounds. For the quinolinone system where Z¹ and Z² together are CH═CH in formula (i), Scheme 5 may be employed:

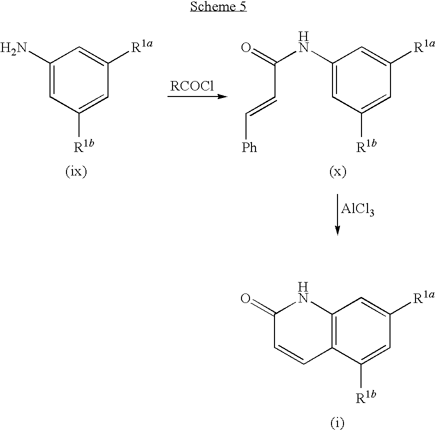

The aniline (ix) is converted to the cinnamide (x), which is cyclised with aluminium chloride (with loss of the phenyl moiety—See M. C. Elliot et al. J. Med. Chem. 47 (22), 5405-5417 (2004), S. R. Inglis et al. Synlett, 5, 898-900 (2004) or Cottet, F.; Marull, M.; Lefebvre, O.; Schlosser, M European Journal of Organic Chemistry (2003), 8, 1559) to give (i).

For the benzothiazole-2-one system (Z¹ and Z² together represent S in formula (i)), conventional methods of synthesis can be used, such as have been reviewed (H. Ulrich and C T Guilford, Science of Synthesis, 2002, 11, 835). In particular, the general 1-pot synthesis of benzothiazol-2-ones described by K. Konichi et al (Synthesis, 1984, 254) can be used.

Compounds of formula (III) HA-N(R²⁰)R^{2′} are known compounds or may be prepared analogously to known compounds, see for example WO2004/035569, WO2004/089947, WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2003082835, WO2002026723, WO06002047, WO06014580, WO06134378, WO06137485, WO07016610, WO07081597, WO07071936, WO07115947, WO07118130, WO07122258, WO08006648, WO08003690 and WO08009700.

Further details for the preparation of compounds of formula (I) are found in the examples.

The antibacterial/antituberculosis compounds according to the invention may be formulated for administration in any convenient way for use in human or veterinary medicine, by analogy with other antibacterials/anti-tubercular compounds.

The pharmaceutical compositions of the invention include those in a form adapted for oral, topical or parenteral use and may be used for the treatment of bacterial infection including tuberculosis in mammals including humans.

The composition may be formulated for administration by any route. The compositions may be in the form of tablets, capsules, powders, granules, lozenges, suppositories, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Such a dosage corresponds to 1.5 to 30 mg/kg per day. Suitably the dosage is from 5 to 20 mg/kg per day.

The compound of formula (I) may be the sole therapeutic agent in the compositions of the invention or a combination with other antibacterials, including anti-tuberculosis compounds. If the other antibacterial is a β-lactam then a β-lactamase inhibitor may also be employed.

Compounds of formula (I) may be used in the treatment of bacterial infections caused by a wide range of organisms including both Gram-negative and Gram-positive organisms, such as upper and/or lower respiratory tract infections, skin and soft tissue infections and/or urinary tract infections. Compounds of formula (I) may be also used in the treatment of tuberculosis caused by Mycobacterium tuberculosis. Some compounds of formula (I) may be active against more than one organism. This may be determined by the methods described herein.

The following examples illustrate the preparation of certain compounds of formula (I) and the activity of certain compounds of formula (I) against various bacterial organisms including Mycobacterium tuberculosis.

### EXAMPLES AND EXPERIMENTAL

### General

### Abbreviations in the Examples:

RT=room temperature
S.T.P=standard temperature and pressure
ES=Electrospray mass spectroscopy
LC-MS=Liquid chromatography mass spectroscopy
HPLC=High Performance Liquid Chromatography (Rt refers to retention time)

Certain reagents are also abbreviated herein. DCM refers to dichloromethane, DMSO refers to dimethylsulfoxide, DMF refers to N,N-dimethylformamide, TFA refers to trifluoroacetic acid, THF refers to tetrahydrofuran, TEA refers to triethylamine, Pd/C refers to palladium on carbon catalyst. Boc refers to t-butoxycarbonyl.

Proton nuclear magnetic resonance (¹H NMR) spectra were recorded at 400 or 250 MHz, and chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform and DMSO-d₆ is hexadeuteriodimethylsulfoxide. Mass spectra were obtained using electrospray (ES) ionization techniques. All temperatures are reported in degrees Celsius.

AD mix alpha is prepared by mixing potassium osmate (K₂OsO₄.2H₂O) (0.52 g), (3a,9R,3′″a,4′″b,9′″R)-9,9′-[1,4-phthalazinediylbis(oxy)]bis[6′-(methyloxy)-10,11-dihydrocinchonan][(DHQ)₂PHAL](5.52 g), then adding potassium ferricyanide [K₃Fe(CN)₆](700 g) and powdered potassium carbonate (294 g). This mixture is stirred in a blender for 30 minutes. This provides approximately 1 kg of AD mix alpha, which is commercially available from Aldrich. See K. Barry Sharpless et al, J. Org. Chem., 1992, 57 (10), 2771. AD mix beta is the corresponding mixture prepared with (9S,9′″S)-9,9′-[1,4-phthalazinediylbis(oxy)]bis[6′-(methyloxy)-10,11-dihydrocinchonan][(DHQD)₂PHAL]. Where AD mix alpha/beta is referred to, this is a 1:1 mixture of the alpha and beta mix.

Chiralpak AD and AD-H columns comprise of silica for preparative columns (5 um particle size AD-H and 10 um particle size AD, 21 mm ID×250 mm L; 20 uM particle size AD, 101 mm ID×250 mm L) coated with Amylose tris (3,5-dimethylphenylcarbamate) (Chiral Technologies USA). Measured retention times are dependent on the precise conditions of the chromatographic procedures. Where quoted below in the Examples they are indicative of the order of elution.

Varian Mega Bond Elut SAX cartridge is an ion-exchange column containing a strongly basic resin (quaternary amine) supplied by Varian (USA).

Reactions involving metal hydrides including lithium hydride, lithium aluminium hydride, di-isobutylaluminium hydride, sodium hydride, sodium borohydride, sodium triacetoxyborohydride, are carried out under argon or other inert gas.

As will be understood by the skilled chemist, references to preparations carried out in a similar manner to, or by the general method of, other preparations, may encompass variations in routine parameters such as time, temperature, workup conditions, minor changes in reagent amounts etc.

### Example 1

### 1-({4-[(2,3-dihydro[1,4]dioxino[2,3-c]pyridin-7-ylmethyl)amino]-1-piperidinyl}methyl)-2,3-dihydro-1H,5H-pyrido[3,2,1-ij]quinolin-5-one dihydrochloride

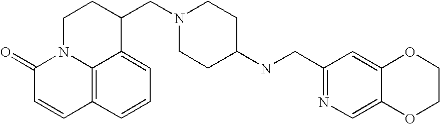

### a) Ethyl 3-(2-oxo-1(2H)-quinolinyl)propanoate

To a suspension of 2-hydroxyquinoline (35 g, 241 mmol), ground KOH (19.5 g, 1.0 equiv), and tetra-n-butyl ammonium bromide (23.25 g, 74.5 mmol) in dry THF (2.0 litre) was added ethyl 3-chloropropionate dropwise with ice-bath cooling. After 30 min the ice-bath was removed and the reaction stirred at room temperature for 42 h. The solvent was evaporated, the residue dissolved in dichloromethane, washed twice with water, dried (MgSO₄) and evaporated. Chromatography on silica gel eluting with 50% ethyl acetate in hexane afforded the desired product as a colourless oil (47.7 g, 81%).

MS (ES+) m/z 246 (MH⁺, 100%), 268 (M+Na, 50%).

### b) 3-(2-oxo-1(2H)-quinolinyl)propanoic acid

Ethyl 3-(2-oxo-1(2H)-quinolinyl)propanoate (10.0 g, 41 mmol) was dissolved in ethanol (150 mL), a solution of sodium hydroxide (3.0 g, 78 mmol) in water (25 mL) added and the mixture heated to reflux for 2 h. The reaction mixture was cooled, evaporated under reduced pressure, and the residue dissolved in water. The solution was washed twice with ethyl acetate and the pH adjusted to 1.0 with 5M HCl. A white precipitate formed which was collected by filtration, washed with water and dried under reduced pressure, to afford the product as a white solid (7.0 g, 79%).

MS (ES+) m/z 218 (MH⁺, 100%), 240 (M+Na, 50%).

### c) 2,3-dihydro-1H,5H-pyrido[3,2,1-ij]quinoline-1,5-dione

A suspension of phosphorus pentoxide (7 g) in polyphosphoric acid (70 g) was heated to 110° C. for 2 hrs with overhead stirring. To the resultant solution was added solid 3-(2-oxo-1(2H)-quinolinyl)propanoic acid, then heating and stirring continued for 20 h. The mixture was poured into ice/water (200 mL), stirred to give a thick precipitate and neutralised with ammonia (aq, 0.880). The resultant precipitate was filtered, dried under reduced pressure, then stirred with 10% methanol in dichloromethane (150 mL) for 30 mins. The mixture was filtered and the filtrate evaporated to dryness to afford the desired compound (4.35 g, 68%).

MS (ES+) m/z 200 (MH⁺, 100%).

### d) 2′,3′-dihydro-5′H-spiro[oxirane-2,1′-pyrido[3,2,1-ij]quinolin]-5′-one

Trimethylsulfoxonium iodide (Aldrich, 0.144 g, 0.65 mmol) was a dissolved in dry DMSO (1.0 mL), cooled in ice, and treated with sodium hydride (60% dispersion in oil, 0.026 g, 0.65 mmol). When effervescence had subsided, the ice bath was removed and the mixture stirred at room temperature for 1 hr. A solution of 2,3-dihydro-1H,5H-pyrido[3,2,1-ij]quinoline-1,5-dione (0.1 g, 0.5 mmol) in dry DMSO (1 mL) was added and the mixture stirred at RT overnight. The resultant red solution was treated cautiously with ice/water and the product extracted into ethyl acetate. The combined organic phases were dried (MgSO4) and evaporated, and the residue chromatographed on silica gel eluting with a gradient of 40-100% ethyl acetate in hexane to afford the desired product as a colourless oil (0.02 g, 20%).

¹H NMR δ(CDCl₃) 2.03 (1H, dt, J 4.0, 13.2 Hz), 2.45 (1H, dt, J 4.4, 12.8 Hz), 3.13 (2H, s), 3.94 (1H, ddd, J 1.6, 3.6, 12.4 Hz), 4.95 (1H, dt, J 4.4, 14.0 Hz), 6.72 (1H, d, J 9.2 Hz), 7.20 (1H, dt, J 7.6 Hz), 7.34 (1H, d, J 7.6 Hz), 7.70 (1H, d, J 9.2 Hz).

MS (ES+) m/z 232 (M+H₂O 100%).

### e) 5-oxo-2,3-dihydro-1H,5H-pyrido[3,2,1-ij]quinoline-1-carbaldehyde

A solution of 2′,3′-dihydro-5′H-spiro[oxirane-2,1′-pyrido[3,2,1-ij]quinolin]-5′-one (0.05 g, 0.23 mmol) in acetone (5 mL) was stirred with acidic alumina (Aldrich, 0.2 g) for 3 days then at 50° C. for 4 h. The mixture was filtered and evaporated to give the crude product as a colourless oil.

MS (ES+) m/z 246 (MH⁺ for methanol hemiacetal, 100%).

### f) Title Compound

5-oxo-2,3-dihydro-1H,5H-pyrido[3,2,1-ij]quinoline-1-carbaldehyde (0.03 g containing approx 0.1 mmol) was dissolved in methanol (3 mL), treated with acetic acid (3 drops), followed by 1,1-dimethylethyl (2,3-dihydro[1,4]dioxino[2,3-c]pyridin-7-ylmethyl)4-piperidinylcarbamate (for a synthesis see WO2004058144 Example 99(h)) (0.035 g). After 10 min the reaction was treated with (polystyrylmethyl)trimethylammonium cyanoborohydride (4.1 mmol/g, 0.2 g), and the mixture stirred at RT overnight. The reaction was filtered and evaporated to an oil. This was dissolved in dichloromethane (1 mL) and TFA (1 mL) and stirred at room temperature for 45 min. The solvent was evaporated and the residue chromatographed on silica gel eluting with a gradient of 1-10% 2M methanolic ammonia in dichloromethane. Product-containing fractions were combined and evaporated to give the free base of the title compound (0.004 g, 9%).

¹H NMR δ(CDCl₃) 1.41-1.52 (2H, m), 1.81-2.04 (4H, m), 2.18 (1H, dt, J 2.4, 11.6 Hz), 2.30-2.2.36 (1H, m), 2.41-2.56 (3H, m), 2.82 (1H, d, J 10.0 Hz), 2.95 (1H, d, J 11.6 Hz), 3.12-3.18 (1H, m), 3.73-3.80 (3H, m), 4.27-4.34 (4H, m), 4.53 (1H, dt, J 4.0, 14.4 Hz), 6.70 (1H, d, J 9.6 Hz), 6.81 (1H, s), 7.14 (1H, t, J 7.6 Hz), 7.41-7.43 (2H, m), 7.67 (1H, d, J 9.6 Hz), 8.10 (1H, s).

MS (ES+) m/z 469(M+Na, 15%), 447 (MH⁺, 40%) and 150 (100%).

The free base of the title compound was dissolved in methanol (0.5 ml), treated with 1M HCl in ether (0.3 ml) and the solution stirred for 20 min. The solution was evaporated to dryness and triturated with diethyl ether to give the title dihydrochloride as a pale yellow solid (4.0 mg)

### Example 2

### 1-({4-[(2,3-dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one hydrochloride

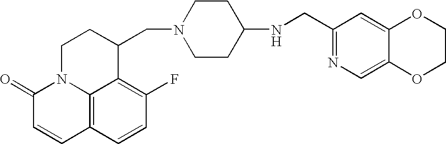

### a) (2E)-N-(3-fluorophenyl)-3-phenyl-2-propenamide

Cinnamoyl chloride (3.6 g, 18 mmol) in ethyl acetate (14 mL) was added to a stirred mixture containing 3-fluoroaniline, ethyl acetate (28 mL) and saturated NaHCO₃ solution (28 mL) and ice (15 g) and stirred for 2 h. The organic layer was then separated and washed with 1N HCl then saturated brine and dried. Chromatography on silica gel eluting with 40% ethyl acetate/40-60 petroleum ether gave the title compound as a white solid (4.14 g, 95%).

MS (ES+) m/z 242 (MH⁺, 100%).

### b) Mixture of 7-fluoro-2(1H)-quinolinone and 5-fluoro-2(1H)-quinolinone

(2E)-N-(3-Fluorophenyl)-3-phenyl-2-propenamide (3.82 g, 15.8 mmol) in chlorobenzene (25 mL) was treated with aluminium trichloride (10.6 g, 79 mmol) portionwise over a 10 min period. The mixture was then heated to 125° C. for 3 h. The mixture was allowed to cool slightly then poured onto ice/water (200 mL) then extracted with 10% methanol/ethyl acetate (2×250 mL) and dried, filtered and evaporated to a small volume giving a pink solid which was filtered off then recrystalised from ethyl acetate/methanol to give mainly the 7-fluoro isomer (1.15 g, 44%). ¹H NMR δ(DMSO-d₆) 6.45 (1H, d, J 9.6 Hz), 6.97-7.07 (1H, m), 7.70-7.77 (1H, m), 7.91 (1H, d, J 9.6 Hz), 11.75-11.91 (1H, brs). The mother liquors were evaporated to dryness to give a 1:1 mixture of the 7-fluoro and 5-fluoro isomers (1 g, 39%).

¹H NMR δ(DMSO-d₆) 6.45 (1H, d, J 9.6 Hz), 6.56 (1H, d, J 9.6 Hz), 6.97-7.07 (1H, m), 7.13 (1H, d, J 7 Hz), 7.45-7.53 (1H, m), 7.70-7.77 (1H, m), 7.91 (1H, d, J 9.6 Hz), 8.01 (1H, d, J 9.6 Hz), 11.75-11.91 (1H, brs), 11.92-12.03 (1H, brs).

### c) Ethyl 3-(7-fluoro-2-oxo-1(2H)-quinolinyl)propanoate and ethyl 3-(5-fluoro-2-oxo-1(2H)-quinolinyl)propanoate

A 1:1 mixture of 7-fluoro-2(1H)-quinolinone and 5-fluoro-2(1H)-quinolinone (7.2 g, 44 mmol) in toluene (140 mL) was treated with cesium fluoride (0.72 g, 4.7 mmol), ethyl acrylate (5.1 mL, 45 mmol) and tetraethyl orthosilicate (9.9 mL, 44 mmol) and heated at 75° C. for 18 h. The resulting mixture was partitioned between ethyl acetate and water and the organic layer dried. Chromatography on silica gel eluting with a gradient of 30-80% ethyl acetate/40-60 petroleum ether gave ethyl 3-(5-fluoro-2-oxo-1(2H)-quinolinyl)propanoate (least polar isomer), (2.63 g).

¹H NMR δ(CDCl₃) 1.23 (3H, t, J 7.2 Hz), 2.71-2.77 (2H, m), 4.17 (2H, q, J 7.2 Hz), 4.55-4.61 (2H, m), 6.7 (1H, d, J 9.6 Hz), 6.89-6.95 (1H, m), 7.21 (1H, d, J 8.8 Hz), 7.47-7.52 (1H, m), 7.97 (1H, d, J 9.6 Hz) and ethyl 3-(7-fluoro-2-oxo-1(2H)-quinolinyl)propanoate (most polar isomer) (4.03 g) ¹H NMR δ (CDCl₃) 1.25 (3H, t, J 7.2 Hz), 2.75-2.80 (2H, m), 4.17 (2H, q, J 7.2 Hz), 4.48-4.57 (2H, m), 6.61 (1H, d, J 9.2 Hz), 6.91-6.97 (1H, m), 7.11-7.17 (1H, m), 7.51-7.57 (1H, m), 7.63 (1H, d, J 9.2 Hz).

### d) 3-(7-Fluoro-2-oxo-1(2H)-quinolinyl)propanoic acid

Ethyl 3-(7-fluoro-2-oxo-1(2H)-quinolinyl)propanoate (5.37 g, 20 mmol) in ethanol (60 mL) was treated with 2M sodium hydroxide solution (20 mL) and stirred at RT for 18 h. The ethanol was then removed in-vacuo and the mixture acidified with 5N hydrochloric acid to pH 1. The resulting white solid was then filtered off and dried in-vacuo to give the title compound (4.29 g, 89%).

MS (ES+) m/z 236 (MH⁺, 100%).

### e) 10-fluoro-2,3-dihydro-1H,5H-pyrido[3,2,1-ij]quinoline-1,5-dione

3-(7-Fluoro-2-oxo-1(2H)-quinolinyl)propanoic acid (4.3 g, 18.3 mmol) was suspended in dry dichloromethane (150 mL), and treated with oxalyl chloride (2.4 mL, 27.5 mmol) followed by 15 drops of dry DMF. After 30 minutes, the reaction had formed a homogeneous solution. The reaction mixture was evaporated to dryness. The crude acid chloride was re-dissolved in dry dichloromethane (150 mL) and treated with aluminium trichloride, (9.8 g, 73.2 mmol). The reaction mixture was left stirring for 48 hrs, then poured into ice/water (400 mL) with stirring and neutralised with solid NaHCO₃. Dichloromethane was added to the stirred mixture followed by Kieselguhr. The resulting mixture was filtered and the filtrate extracted with dichloromethane. The organic layer was dried over anhydrous magnesium sulphate to give the desired compound as a yellow solid, (3.18 g, 80%).

¹H NMR δ(CDCl₃) 2.89-2.96 (2H, m), 4.53-4.60 (2H, m), 6.72 (1H, d, J 10 Hz), 6.98-7.06 (1H, m), 7.68-7.77 (2H, m).

MS (ES+) m/z 218 (MH⁺, 100%).

### f) 10-Fluoro-1-[(methyloxy)methylidene]-2,3-dihydro-1H,5H-pyrido[3,2,1-ij]quinolin-5-one

Methoxymethylenetriphenyl phosphonium chloride (16.75 g, 48.8 mmol) was suspended in dry 1,4-dioxane (140 mL), cooled in ice, and treated with potassium tert-butoxide (5.45 g, 48.8 mmol). The cooling bath was removed and the mixture stirred for a further 30 min. A solution of 10-fluoro-2,3-dihydro-1H,5H-pyrido[3,2,1-ij]quinoline-1,5-dione (2.13 g, 9.77 mmol) in dry 1,4-dioxane was added and the mixture stirred at room temperature for 30 min. Water (500 mL) was added and the resultant solution extracted with ethyl acetate (2×500 mL). The combined organic phases were dried over anhydrous magnesium sulphate and evaporated. The crude product was chromatographed on silica gel eluting with 1-100% ethyl acetate in hexane. The product was obtained as a pale yellow solid (0.81 g, 33%).

MS (ES+) m/z 246 (MH⁺, 100%).

### g) 10-Fluoro-5-oxo-2,3-dihydro-1H,5H-pyrido[3,2,1-ij]quinoline-1-carbaldehyde

10-Fluoro-1-[(methyloxy)methylidene]-2,3-dihydro-1H,5H-pyrido[3,2,1-ij]quinolin-5-one (0.7 g, 2.86 mmol) was added to a solution of anhydrous sodium iodide (0.448 g, 2.9 mmol) in dry acetonitrile (50 mL) at room temperature under an argon atmosphere. Trimethylchlorosilane (0.38 mL, 2.86 mmol) was added dropwise, and the mixture stirred for 40 min. The solution was diluted with ethyl acetate and washed with 10% sodium thiosulphate solution, water, and saturated aqueous sodium chloride. The organic phase was dried over anhydrous magnesium sulphate, and evaporated to give the crude title compound as an oil (0.67 g). 0.2 g of this was purified by chromatography on silica gel eluting with 30-100% ethyl acetate in hexane to afford the pure product as a colourless oil (0.13 g).

MS (ES+) m/z 232 (MH⁺, 100%).

### h) Title Compound

10-Fluoro-5-oxo-2,3-dihydro-1H,5H-pyrido[3,2,1-ij]quinoline-1-carbaldehyde (0.558 g, 2.38 mmol) was dissolved in methanol (95 mL), treated with acetic acid (0.95 mL), followed by 1,1-dimethylethyl (2,3-dihydro[1,4]dioxino[2,3-c]pyridin-7-ylmethyl)4-piperidinylcarbamate (0.832 g) (for a synthesis see WO2004058144 Example 99(h)). After 10 min the reaction was treated with (polystyrylmethyl)trimethylammonium cyanoborohydride (4.1 mmol/g, 2.4 g), and the mixture stirred at room temperature for 4 days. The reaction was filtered, the resin washed with methanol, and the filtrate evaporated. The residue was chromatographed on silica gel eluting with a gradient of 1-10% 2M methanolic ammonia in dichloromethane. Product-containing fractions were combined and evaporated to give a pale yellow oil containing some impurity (total 0.77 g). This was dissolved in dichloromethane (7 mL), treated with TFA (7 mL) and the mixture stirred at room temperature for 30 min, then evaporated to dryness. The residue was chromatographed on silica gel eluting with a gradient of 1-10% 2M methanolic ammonia in dichloromethane. Product-containing fractions were combined and evaporated to give the free base of the title compound as a pale yellow foam (0.446 g, 0.96 mmol, 40%).

¹H NMR δ(CDCl₃) 1.51-1.82 (4H, m), 1.95-2.10 (3H, m), 2.20-2.40 (1H, m), 2.45-2.57 (4H, m), 2.80-2.83 (2H, m), 3.12 (1H, d, J 11.2 Hz), 3.44-3.52 (1H, m), 4.00 (2H, s), 4.27-4.35 (4H, m), 4.76 (1H, dd, J 3.8, 14.9 Hz), 6.64 (1H, d, J 9.6 Hz), 6.84 (1H, s), 6.92 (1H, t, J 8.8 Hz), 7.38-7.41 (2H, m), 7.62 (1H, d, J 9.6 Hz), 8.10 (1H, s).

MS (ES+) m/z 465 (MH⁺, 35%).

The free base of the title compound was dissolved in DCM and converted to the hydrochloride by addition of one equivalent of 1.0M hydrogen chloride in ether to give the title compound (0.022 g).

### Example 3

### 1-({4-[(2,3-dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one dihydrochloride (Enantiomer 1) and

### Example 4

### 1-({4-[(2,3-dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one dihydrochloride (Enantiomer 2)

Racemic 1-({4-[(2,3-dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one (total of 0.386 g) was chromatographed in 4 batches on a Chiralpak AD 21×250 mm column, eluting with a mixture of 50:50:0.1 acetonitrile: methanol: isopropylamine at 20 ml/min. The peak with retention time of 10.2 min was collected evaporated to give Enantiomer 1 (0.136 g) and the peak with retention time 20.3 min collected and evaporated to give Enantiomer 2 (0.149 g).

The free bases were slurried separately in methanol (5 mL) and treated with 2.0 equiv. of aqueous 6M HCl. The solvent was removed under reduced pressure and the solids dried under reduced pressure at 40° C. to give the corresponding title dihydrochloride salts, each with >99% ee.

### Example 5

### 1-({4-[(2,3-dihydro[1,4]dioxino[2,3-c]pyridin-7-ylmethyl)amino]-1-piperidinyl}methyl)-8-fluoro-2,3-dihydro-1H,5H-pyrido[3,2,1-ij]quinolin-5-one hydrochloride

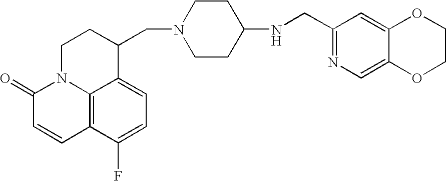

### a) 3-(5-Fluoro-2-oxo-1(2H)-quinolinyl)propanoic acid

The title compound was prepared by the general method of Example 2d) from ethyl 3-(5-fluoro-2-oxo-1(2H)-quinolinyl)propanoate (3.37 g, 12.8 mmol) to give (2.85 g, 94%).

MS (ES+) m/z 236 (MH⁺, 100%).

### b) 8-fluoro-2,3-dihydro-1H,5H-pyrido[3,2,1-ij]quinoline-1,5-dione

3-(5-Fluoro-2-oxo-1(2H)-quinolinyl)propanoic acid (3.26 g, 13.8 mmol) was suspended in dichloromethane, treated with oxalyl chloride (1.27 mL, 13.8 mmol) followed by DMF (10 drops). After 40 min, the reaction was cooled in ice, and treated with aluminium trichloride (7.4 g) portionwise over 10 min. The reaction mixture was allowed to warm to room temperature and stirred for 18 h. The mixture was poured into ice (˜300 mL), cautiously neutralised with solid sodium bicarbonate, then extracted with dichloromethane. The organic extracts were combined, dried over anhydrous magnesium sulphate and evaporated to give the desired product as an off-white solid, (2.42 g, 80%).

¹H NMR δ(CDCl₃) 2.92-2.95 (2H, m), 4.54-4.58 (2H, m), 6.77 (1H, d, J 10 Hz), 7.01 (1H, t, J 8.8 Hz), 7.97 (1H, d, J 10 Hz), 8.17-8.22 (1H, m).

MS (ES+) m/z 218 (MH⁺, 100%).

### c) 8-fluoro-1-[(methyloxy)methylidene]-2,3-dihydro-1H,5H-pyrido[3,2,1-ij]quinolin-5-one

8-Fluoro-2,3-dihydro-1H,5H-pyrido[3,2,1-ij]quinoline-1,5-dione (0.0184 g, 0.84 mmol) was converted into the enol ether by the general method of Example 2f) in 30% yield (0.06 g).

MS (ES+) m/z 246 (MH⁺, 100%).

### d) 8-Fluoro-5-oxo-2,3-dihydro-1H,5H-pyrido[3,2,1-ij]quinoline-1-carbaldehyde

8-Fluoro-1-[(methyloxy)methylidene]-2,3-dihydro-1H,5H-pyrido[3,2,1-ij]quinolin-5-one (0.06 g, 0.24 mmol) was converted into the title compound by the general method of Example 2g), with a reaction time of 20 min. After work-up the crude product was obtained as a pale yellow oil (0.054 g, 100%).

¹H NMR δ(CDCl₃) 2.62-2.68 (1H, m), 3.68-75 (1H, m), 3.70-3.81 (1H, m), 4.54-4.61 (1H, m), 6.77 (1H, d, J 10 Hz), 7.01 (1H, t, J 8.8 Hz), 7.36-7.40 (1H, m), 7.97 (1H, d, J 10 Hz), 9.79 (1H, s).

MS (ES+) m/z 232 (MH⁺, 100%).

### e) Title Compound

The title compound was prepared by the general method of Example 1f) from 8-fluoro-5-oxo-2,3-dihydro-1H,5H-pyrido[3,2,1-ij]quinoline-1-carbaldehyde (0.05 g, 0.22 mmol) and 1,1-dimethylethyl (2,3-dihydro[1,4]dioxino[2,3-c]pyridin-7-ylmethyl)4-piperidinylcarbamate (0.075 g, 0.22 mmol) (for a synthesis see WO2004058144 Example 99(h)) to give the free base of the title compound (0.029 g, 28%).

¹H NMR δ(CDCl₃) 1.40-1.61 (2H, m), 1.75-2.08 (4H, m), 2.09-2.16 (1H, m), 2.23-2.32 (1H, m), 2.37-2.49 (2H, m), 2.50-2.60 (1H, m), 2.78-2.88 (1H, m), 2.89-2.96 (1H, m), 3.05-3.14 (1H, m), 3.69-3.78 (1H, m), 3.80 (2H, s), 4.25-4.38 (4H, m), 4.44-4.52 (1H, m), 6.71 (1H, d, J 9.6 Hz), 6.80-6.85 (2H, m), 7.31-7.40 (1H, m), 7.93 (1H, d, J 9.6 Hz), 8.10 (1H, s).

MS (ES+) m/z 465 (MH⁺, 40%).

The free base of the title compound was dissolved in dichloromethane (3 mL), treated with 1M HCl in ether (0.075 mL) and the solution stirred for several seconds. The solution was evaporated to dryness to give (0.036 g) of the mono hydrochloride salt.

### Example 6

### 1-({4-[(2,3-dihydro[1,4]dioxino[2,3-c]pyridin-7-ylmethyl)amino]-1-piperidinyl}methyl)-8,10-difluoro-2,3-dihydro-1H,5H-pyrido[3,2,1-ij]quinolin-5-one dihydrochloride

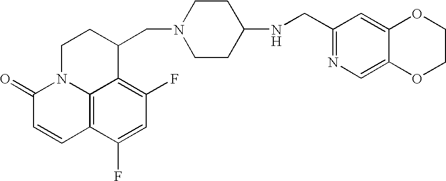

### a) (2E)-N-(3,5-difluorophenyl)-3-phenyl-2-propenamide

The title compound was prepared by the general method of Example 2a) from 3,5-difluoroaniline (15 g, 116 mmol) to give (30 g, 100%).

MS (ES+) m/z 260 (MH⁺, 100%).

### b) 5,7-Difluoro-2(1H)-quinolinone

The title compound was prepared by the general method of Example 2b) from (2E)-N-(3,5-difluorophenyl)-3-phenyl-2-propenamide (30 g, 116 mmol) to give the title compound (0.5 g, 2.4%).

MS (ES+) m/z 182 (MH⁺, 100%).

### c) Ethyl 3-(5,7-difluoro-2-oxo-1(2H)-quinolinyl)propanoate

The title compound was prepared by the general method of Example 2c) from 5,7-difluoro-2(1H)-quinolinone (0.5 g, 2.7 mmol) to give (0.53 g, 68%).

MS (ES+) m/z 282 (MH⁺, 35%), 304 (M+Na, 65%).

### d) 3-(5,7-Difluoro-2-oxo-1(2H)-quinolinyl)propanoic acid

The title compound was prepared by the general method of Example 2d) from ethyl 3-(5,7-difluoro-2-oxo-1(2H)-quinolinyl)propanoate (0.53 g, 1.9 mmol) to give (0.279 g, 59%).

MS (ES+) m/z 254 (MH⁺, 75%).

### e) 8,10-Difluoro-2,3-dihydro-1H,5H-pyrido[3,2,1-ij]quinoline-1,5-dione

The title compound was prepared by the general method of Example 2e) from 3-(5,7-difluoro-2-oxo-1(2H)-quinolinyl)propanoic acid (0.233 g, 0.92 mmol) to give (0.196 g, 90%).

MS (ES+) m/z 236 (MH⁺, 100%).

### f) 8,10-Difluoro-1-[(methyloxy)methylidene]-2,3-dihydro-1H,5H-pyrido[3,2,1-ij]quinolin-5-one

The title compound was prepared by the general method of Example 2f) from 8,10-difluoro-2,3-dihydro-1H,5H-pyrido[3,2,1-ij]quinoline-1,5-dione (0.16 g, 0.68 mmol) to give (0.024 g, 13%).

MS (ES+) m/z 264 (MH⁺, 100%).

### g) 8,10-Difluoro-5-oxo-2,3-dihydro-1H,5H-pyrido[3,2,1-ij]quinoline-1-carbaldehyde

The title compound was prepared by the general method of Example 2g) from 8,10-difluoro-1-[(methyloxy)methylidene]-2,3-dihydro-1H,5H-pyrido[3,2,1-ij]quinolin-5-one (0.024 g, 0.09 mmol) to give (0.018 g, 79%).

MS (ES+) m/z 282 (MH⁺ for methanol hemiacetal, 100%).

### h) Title Compound

The free base of the title compound was prepared by the general method of Example 1f) from 8,10-difluoro-5-oxo-2,3-dihydro-1H,5H-pyrido[3,2,1-ij]quinoline-1-carbaldehyde (0.01 g, 0.004 mmol) and 1,1-dimethylethyl (2,3-dihydro[1,4]dioxino[2,3-c]pyridin-7-ylmethyl)4-piperidinylcarbamate (0.014 g, 0.004 mmol) (for a synthesis see WO2004058144 Example 99(h)) to give product (0.006 g, 31%).

¹H NMR δ(CDCl₃) 1.38-1.85 (6H, m), 1.99-2.06 (1H, m), 2.15-2.21 (1H, m), 2.35-2.48 (2H, m), 2.51-2.59 (1H, m), 2.65-2.73 (1H, m), 2.74-2.80 (1H, m), 3.02-3.09 (1H, m), 3.38-3.45 (1H, m), 3.90 (2H, s), 4.25-4.38 (4H, m), 4.69-4.77 (1H, m), 6.63-6.69 (2H, m), 6.82 (1H, s), 7.88 (1H, d, J 9.6 Hz), 8.10 (1H, s). MS (ES+) m/z 483 (MH⁺, 35%).

This was dissolved in methanol and treated with excess 1M HCl in ether and the solution stirred for several seconds. The solution was evaporated to dryness to give (0.007 g) of the dihydrochloride salt.

### Example 7

### 1-({4-[(6,7-dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one hydrochloride (Enantiomer 1)

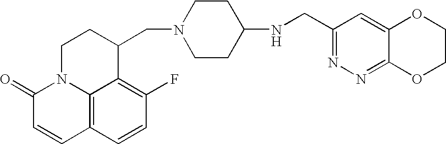

### a) 1-[(4-Amino-1-piperidinyl)methyl]-10-fluoro-2,3-dihydro-1H,5H-pyrido[3,2,1-ij]quinolin-5-one (racemate)

The title compound was prepared by the general method of Example 1f) from 10-fluoro-5-oxo-2,3-dihydro-1H,5H-pyrido[3,2,1-ij]quinoline-1-carbaldehyde (for a preparation see Example 2g)) (0.758 g, 3.28 mmol) and 4-(N-Boc-amino)piperidine (0.656 g, 3.28 mmol) to give the racemic Boc protected intermediate product (0.392 g). This was deprotected in the usual manner using TFA and a basic workup to give (0.232 g).

¹H NMR δ(CDCl₃) 1.38-1.52 (2H, m), 1.71-1.91 (3H, m), 1.98-2.05 (1H, m), 2.17-2.28 (1H, m), 2.37-2.61 (5H, m), 2.68-2.75 (2H, m), 3.02-3.10 (1H, m), 3.43-3.52 (2H, m), 4.70-4.80 (1H, m), 6.64 (1H, d, J 9.6 Hz), 6.89-6.95 (1H, m), 7.35-7.43 (1H, m), 7.65 (1H, d, J 9.6 Hz).

### b) Resolution by chiral HPLC to give 1-[(4-Amino-1-piperidinyl)methyl]-10-fluoro-2,3-dihydro-1H,5H-pyrido[3,2,1-ij]quinolin-5-one (Enantiomer 1) and (Enantiomer 2)

1-[(4-Amino-1-piperidinyl)methyl]-10-fluoro-2,3-dihydro-1H,5H-pyrido[3,2,1-ij]quinolin-5-one (racemate) (0.232 g) was resolved by chiral HPLC using a Chiralpak AD-H column (21×250 mm), mobile phase A was 0.1% isopropylamine in acetonitrile and mobile phase B was 0.11% isopropylamine in methanol. The column was run isocratically using 15% mobile phase B in mobile phase A over multiple injections, this gave (0.045 g) Enantiomer 1, retention time 4.6 mins and (0.054 g) Enantiomer 2, retention time 8.5 mins.

### c) 3,4,6-Trichloropyridazine

This was prepared by a slight variation on the method of Kasnar et al, Nucleosides & Nucleotides (1994), 13(1-3), 459-79.

Hydrazine sulphate salt (51 g) was suspended in water (250 ml), heated to reflux and bromomaleic anhydride (90.38 g) was added dropwise. The mixture was heated at reflux for 4 hours then cooled to room temperature. The reaction was repeated with 29 g hydrazine sulphate, 53 g bromomaleic anhydride and 130 ml water. The precipitates were collected by filtration, washed with water and acetone and dried as a combined batch in vacuo to afford 4-bromo-1,2-dihydro-3,6-pyridazinedione as a white solid (113 g). The solid in two batches was treated with phosphorus oxychloride (2×200 ml) and heated to reflux for 3.5 hours. The mixture was cooled, evaporated and azeotroped with toluene. The residue was partitioned between dichloromethane and saturated aqueous sodium bicarbonate solution and extracted with DCM twice more. The organic extracts were dried and evaporated. This residue was re-dissolved in dichloromethane, and chromatographed on silica gel (300 g) (DCM as eluent) to give a white solid (101.5 g, 87%).

(LC-MS analysis showed ca 20-30% impurity, isomers of bromo-dichloropyridazine).

MS (+ve ion electrospray) m/z 184/185/186 (MH+), trichloropyridazine.

MS (+ve ion electrospray) m/z 228/229/231 (MH+), bromo-dichloropyridazine.

### d) 2-[(3,6-Dichloro-4-pyridazinyl)oxy]ethanol

A solution of ethylene glycol (55 ml) in tetrahydrofuran (200 ml) was treated at around 0° C. (ice bath cooling) with sodium hydride (60% dispersion in oil, 5.9 g) over 40 minutes. After the addition was complete, 3,4,6-trichloropyridazine (27 g) containing isomers of bromo-dichloropyridazine as impurity was added portionwise and washed in with more dry THF (50 ml) and the mixture was stirred at 0° C. for 1 hour and then at room temperature overnight. The mixture was concentrated (to 1/3 volume) then diluted with aqueous sodium bicarbonate solution and extracted with chloroform (5×) and ethyl acetate (3×). The combined organic extracts were washed with water, dried over sodium sulphate and evaporated and the solids filtered off and washed with CHCl₃ (×3) and dried in a vacuum oven overnight at 40° C. affording a white solid (25.5 g, 83%), containing some bromo-derivative (10-15%).

MS (+ve ion electrospray) m/z 209/211 (MH+).

MS (+ve ion electrospray) m/z 255/7 (MH+), bromo-derivative.

### e) 3-Chloro-6,7-dihydro[1,4]dioxino[2,3-c]pyridazine

A solution of 2-[(3,6-dichloro-4-pyridazinyl)oxy]ethanol containing some bromo-derivative (15.46 g; 0.0703 mol) in dry 1,4-dioxane (1.2 L) was treated with lithium hydride (2.3 g; 0.28 mol) in portions and stirred at room temperature for 1 hour under argon, then heated at 110° C. overnight. The reaction mixture was quenched with wet 1,4-dioxane, then iced-water. The solution was evaporated to half volume, taken to pH 8 with 5M hydrochloric acid and evaporated to dryness. Water was added and the residue was extracted 5× with chloroform, dried (sodium sulphate) and evaporated to afford a white solid (12.4 g, ca.77%) (containing ca. 15% of a bromo species).

MS (+ve ion electrospray) m/z 173/5 (Cl MH+); 217/9 (Br MH+)

### f) 3-Ethenyl-6,7-dihydro[1,4]dioxino[2,3-c]pyridazine

A solution of 3-chloro-6,7-dihydro[1,4]dioxino[2,3-c]pyridazine (13.6 g, 0.079 mol) containing ca. 15% of a bromo species in dimethoxyethane (400 ml) was degassed under argon for 10 min then tetrakis(triphenylphosphine)palladium (0) (2 g), potassium carbonate (10.33 g), 2,4,6-trivinylcyclotriboroxane pyridine complex (11.32 g) and water (55 ml) were added. The mixture was heated at 95° C. for 48 hours and cooled and evaporated to dryness. The mixture was treated with aqueous sodium bicarbonate solution and extracted (5×) with DCM. Extracts were dried (sodium sulphate), evaporated and the residue chromatographed on silica gel (500 g), eluting with 0-100% ethyl acetate-hexane, affording the product (6.43 g, 50%); [also some impure fractions (1.8 g)].

MS (+ve ion electrospray) m/z 165 (MH+).

### g) 6,7-Dihydro[1,4]dioxino[2,3-c]pyridazine-3-carbaldehyde

A solution of 3-ethenyl-6,7-dihydro[1,4]dioxino[2,3-c]pyridazine (11.58 g) in 1,4-dioxane/water (600 ml/180 ml), cooled in ice, was treated with an aqueous solution of osmium tetroxide (4% w/v, 25 ml) and sodium periodate (43 g). This mixture was allowed to warm to room temperature and after 7 hours under stirring the mixture was evaporated to dryness and azeotroped with 1,4-dioxane. Silica gel, 1,4-dioxane and chloroform were added and the mixture was evaporated to dryness overnight, then added to a silica column (400 g) and chromatographed, eluting with chloroform then 0-100% ethyl acetate in hexane, to afford a white solid (7.55 g, 64%).

MS (+ve ion electrospray) m/z 167 (MH+).

### h) Title Compound

1-[(4-Amino-1-piperidinyl)methyl]-10-fluoro-2,3-dihydro-1H,5H-pyrido[3,2,1-ij]quinolin-5-one (Enantiomer 1) (0.045 g, 0.14 mmol) and 6,7-dihydro[1,4]dioxino[2,3-c]pyridazine-3-carbaldehyde (0.024 g, 0.14 mmol) were dissolved in dichloromethane (5 mL) and methanol (4 mL) and treated with acetic acid (2 drops) and (polystyrylmethyl)trimethylammonium cyanoborohydride (4.1 mmol/g, 0.14 g) and stirred at RT for 18 hrs. The mixture was then filtered and the filtrate evaporated to dryness. Chromatography on silica gel eluting with a gradient of 2-10% 2M methanolic ammonia in dichloromethane to give the free base of the title compound (0.056 g, 85%).

¹H NMR δ(CDCl₃) 1.35-1.51 (2H, m), 1.72-1.82 (1H, m), 1.83-1.95 (1H, m), 1.96-2.05 (1H, m), 2.15-2.23 (1H, m), 2.35-2.51 (5H, m), 2.71-2.78 (1H, m), 3.03-3.11 (1H, m), 3.42-3.53 (2H, m), 4.01 (2H, s), 4.36-4.39 (2H, m), 4.49-4.54 (2H, m), 4.71-4.78 (1H, m), 6.61 (1H, d, J 9.6 Hz), 6.88-6.93 (1H, m), 7.05 (1H, s), 7.35-7.41 (1H, m), 7.63 (1H, d, J 9.6 Hz).

MS (ES+) m/z 466 (MH⁺, 25%).

The free base of the title compound was dissolved in dichloromethane (3.5 mL) and treated with 1M HCl in ether (0.41 mL) and the solution stirred for several seconds. The solution was evaporated to dryness to give the title hydrochloride salt (0.062 g).

### Example 8

### 1-({4-[(6,7-dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one hydrochloride (Enantiomer 2)

The title compound was prepared from 1-[(4-Amino-1-piperidinyl)methyl]-10-fluoro-2,3-dihydro-1H,5H-pyrido[3,2,1-ij]quinolin-5-one (Enantiomer 2) (for a preparation see Example 7b)) (0.054 g, 0.17 mmol) and 6,7-dihydro[1,4]dioxine[2,3-c]pyridazine-3carbaldehyde (for a preparation see Example 7g)) (0.028 g, 0.17 mmol) (using the general method of Example 7h) to give the free base (0.051 g, 64%).

¹H NMR δ(CDCl₃) 1.35-1.51 (2H, m), 1.72-1.82 (1H, m), 1.83-1.95 (1H, m), 1.96-2.05 (1H, m), 2.15-2.23 (1H, m), 2.35-2.51 (5H, m), 2.71-2.78 (1H, m), 3.03-3.11 (1H, m), 3.42-3.53 (2H, m), 4.01 (2H, s), 4.36-4.39 (2H, m), 4.49-4.54 (2H, m), 4.71-4.78 (1H, m), 6.61 (1H, d, J 9.6 Hz), 6.88-6.93 (1H, m), 7.05 (1H, s), 7.35-7.41 (1H, m), 7.63 (1H, d, J 9.6 Hz).

MS (ES+) m/z 466 (MH⁺, 20%).

The free base of the title compound was dissolved in dichloromethane (3.5 mL) and treated with 1M HCl in ether (0.41 mL) and the solution stirred for several seconds. The solution was evaporated to dryness to give the title hydrochloride salt (0.061 g).

### Example 9

### 1-({4-[(6,7-dihydro[1,4]oxathiino [2,3-c]pyridazin-3-ylmethyl)amino]-1-piperidinyl}methyl)-10-fluoro-2,3-dihydro-1H,5H-pyrido[3,2,1-ij]quinolin-5-one dihydrochloride

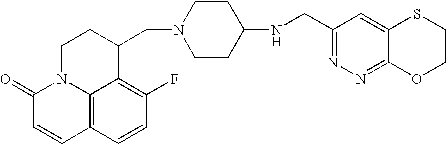

### a) 2-[(3,6-Dichloro-4-pyridazinyl)thio]ethanol

A solution of 3,4,6-trichloropyridazine (25 g) in tetrahydrofuran (200 ml) and triethylamine (19 ml) was treated at 0° C. (ice bath cooling) with 2-mercaptoethanol (8.33 ml) over 5 minutes. After the addition was complete, the mixture was stirred at RT for 72 hours. The mixture was stirred with aqueous sodium bicarbonate solution and DCM and the solid was collected, washed with water, ether and pentane and dried in vacuo, giving (22.9 g). The combined aqueous and organic fraction was evaporated to half volume giving further solid, which was washed and dried as above (5.0 g). The total yield of solid (27.9 g; 91%) contained some bromo-analogue (5-10%) by NMR.

### b) 3-Chloro-6,7-dihydro[1,4]oxathiino[2,3-c]pyridazine

A solution of 2-[(3,6-dichloro-4-pyridazinyl)thio]ethanol (13 g) (previously dried at 50° C. in vacuo) in dry 1,4-dioxane (250 ml) was treated with lithium hydride (3 g) in portions and heated at 105-110° C. for 24 h. The reaction mixture was cooled and quenched with iced-water. The solution was taken to pH 10-11 with 5M hydrochloric acid and evaporated. Water was added and the mixture was extracted 4× with DCM, dried (sodium sulphate), evaporated, and chromatographed on silica gel, eluting with 0-100% ethyl acetate-hexane, to afford a white solid (1.61 g) (containing ca. 10% of the bromo species).

MS (+ve ion electrospray) m/z 189/91 (Cl MH+); 233/5 (Br MH+).

¹H NMR δ(CDCl₃, 400 MHz) 3.23 (2H, m), 4.67 (2H, m), 7.26 (1H, s) (for major chloro-compound).

### c) 3-Ethenyl-6,7-dihydro[1,4]oxathiino[2,3-c]pyridazine

A solution of 3-chloro-6,7-dihydro[1,4]oxathiino[2,3-c]pyridazine (1.0 g) in dimethoxyethane (2 ml) was degassed under argon then tetrakis(triphenylphosphine)palladium (0) (135 mg), potassium carbonate (0.695 g), triethenylboroxin pyridine complex (0.8 g) and water (3.7 ml) were added. The mixture was heated overnight at 105° C. More triethenylboroxin pyridine complex (0.4 g) and tetrakis(triphenylphosphine)palladium (0) (30 mg) were added and heating was continued for 24 hours. The mixture was cooled, treated with aqueous sodium bicarbonate solution, extracted (4×) with DCM, dried (sodium sulphate), evaporated and chromatographed on silica gel (70 g), eluting with 0-100% ethyl acetate-hexane, affording a solid (0.56 g) (87% pure by LC-MS).

MS (+ve ion electrospray) m/z 181 (MH+).

### d) 6,7-Dihydro[1,4]oxathiino[2,3-c]pyridazine-3-carbaldehyde

A solution of 3-ethenyl-6,7-dihydro[1,4]oxathiino[2,3-c]pyridazine (320 mg) in 1,4-dioxane/water (20 ml/5 ml) was treated with an aqueous solution of osmium tetroxide (4% w/v, 2 ml) and sodium periodate (1.0 g), initially stirred in an ice-bath, then allowed to warm to RT. After 2.5 h the mixture was evaporated to dryness and dissolved in 1,4-dioxane and chloroform. Silica gel was added and the mixture was evaporated to dryness, added to a silica column (50 g) and chromatographed, eluting with 0-100% ethyl acetate in hexane, to afford a white solid (116 mg, 36%).

MS (+ve ion electrospray) m/z 183 (MH+).

### e) Title Compound

The title compound was prepared by the general method of Example 7h) from racemic 1-[(4-amino-1-piperidinyl)methyl]-10-fluoro-2,3-dihydro-1H,5H-pyrido[3,2,1-ij]quinolin-5-one (for a preparation see Example 7a)) (0.05 g, 0.16 mmol) and 6,7-dihydro[1,4]oxathiino[2,3-c]pyridazine-3-carbaldehyde (0.029 g, 0.16 mmol) to give the free base as a solid (0.044 g, 58%).

¹H NMR δ(CDCl₃) 1.35-1.51 (2H, m), 1.72-2.02 (4H, m), 2.15-2.38 (1H, m), 2.35-2.71 (6H, m), 3.05-3.15 (1H, m), 3.20-3.25 (2H, m), 3.42-3.53 (2H, m), 4.01 (2H, s), 4.63-4.65 (2H, m), 4.71-4.81 (1H, m), 6.65 (1H, d, J 9.5 Hz), 6.89-6.96 (1H, m), 7.37-7.43 (2H, m), 7.64 (1H, d, J 9.5 Hz).

MS (ES+) m/z 482 (MH⁺, 100%).

The free base of the title compound was dissolved in methanol, treated with 1 M HCl in ether (0.27 mL, 3 equiv.) and the solution stirred for several seconds. The solution was evaporated to dryness to give the title dihydrochloride salt.

### Example 10

### 10-fluoro-1-[(4-{[(3-oxo-3,4-dihydro-2H-pyrido[3,2-b][1,4]thiazin-6-yl)methyl]amino}-1-piperidinyl)methyl]-2,3-dihydro-1H,5H-pyrido[3,2,1-ij]quinolin-5-one dihydrochloride

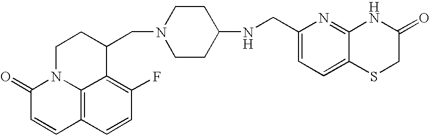

The title compound was prepared by the general method of Example 7h) from racemic 1-[(4-amino-1-piperidinyl)methyl]-10-fluoro-2,3-dihydro-1H,5H-pyrido[3,2,1-ij]quinolin-5-one (for a preparation see Example 7a)) (0.05 g, 0.16 mmol) and 3-oxo-3,4-dihydro-2H-pyrido[3,2-b][1,4]thiazine-6-carboxaldehyde (0.03 g, 0.16 mmol) (for a synthesis see WO2004058144 Example 7(d)) to give the free base as a solid (0.037 g, 47%).

¹H NMR δ(CDCl₃) 1.35-1.52 (2H, m), 1.72-2.22 (6H, m), 2.25-2.35 (1H, m), 2.42-2.64 (4H, m), 2.70-2.83 (1H, m), 3.05-3.15 (1H, m), 3.45-3.52 (4H, m), 3.86 (2H, s), 4.72-4.80 (1H, m), 6.65 (1H, d, J 9.5 Hz), 6.88-7.01 (2H, m), 7.37-7.43 (1H, m), 7.56-7.65 (1H, m).

MS (ES+) m/z 494 (MH⁺, 100%).

The free base of the title compound was dissolved in dichloromethane, treated with 1 M HCl in ether (˜3 eqs. 0.2 mL) and the solution stirred for several seconds. The solution was evaporated to dryness to give the title dihydrochloride salt.

### Example 11

### 10-fluoro-1-[(4-{[(3-oxo-3,4-dihydro-2H-pyrido[3,2-b][1,4]oxazin-6-yl)methyl]amino}-1-piperidinyl)methyl]-2,3-dihydro-1H,5H-pyrido[3,2,1-ij]quinolin-5-one dihydrochloride

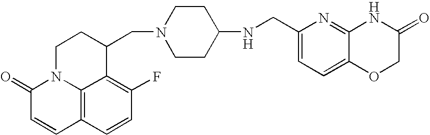

The title compound was prepared by the general method of Example 7h) from racemic 1-[(4-amino-1-piperidinyl)methyl]-10-fluoro-2,3-dihydro-1H,5H-pyrido[3,2,1-ij]quinolin-5-one (for a preparation see Example 7a)) (0.05 g, 0.16 mmol) and 3-oxo-3,4-dihydro-2H-pyrido[3,2-b][1,4]oxazine-6-carboxaldehyde (0.028 g, 0.16 mmol) (for a synthesis see WO2003087898 Example 31(e)) to give the free base as a solid (0.025 g, 32%).

¹H NMR δ(CDCl₃) 1.45-1.62 (2H, m), 1.72-2.22 (6H, m), 2.41-2.62 (4H, m), 2.70-2.90 (1H, m), 3.11-3.23 (1H, m), 3.44-3.60 (3H, m), 3.81-3.92 (1H, s), 4.60-4.63 (2H, m), 4.72-4.83 (1H, m), 6.64 (1H, d, J 9.5 Hz), 6.88-6.97 (2H, m), 7.37-7.42 (1H, m), 7.62 (1H, d, J 9.5 Hz).

MS (ES+) m/z 477 (MH⁺, 100%).

The free base of the title compound was dissolved in DCM, treated with 1 M HCl in ether (˜3 eqs. 0.18 mL) and the solution stirred for several seconds. The solution was evaporated to dryness to give the title dihydrochloride salt.

### Example 12

### 1-({4-[(2,3-dihydro[1,4]dioxino[2,3-c]pyridin-7-ylmethyl)amino]-1-piperidinyl}methyl)-10-(methyloxy)-2,3-dihydro-1H,5H-pyrido[3,2,1-ij]quinolin-5-one dihydrochloride

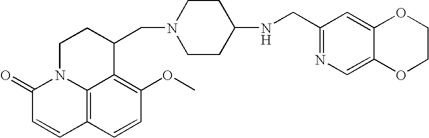

### a) 10-(methyloxy)-2,3-dihydro-1H,5H-pyrido[3,2,1-ij]quinoline-1,5-dione

10-Fluoro-5-oxo-2,3-dihydro-1H,5H-pyrido[3,2,1-ij]quinoline-1-carbaldehyde (0.011 g, 0.5 mmol) was dissolved in dichloromethane (10 mL) and treated with a solution of sodium methoxide (25% w/w in methanol, 0.12 mL, 0.5 mmol) at room temperature under an argon atmosphere. After 10 min, ethyl formate was added and the solution left to stir for 3 days. The reaction mixture was diluted with dichloromethane, and washed with water followed by 1 M sodium hydroxide solution. The organic phase was dried and evaporated to give the product as a yellow solid (0.092 g, 80%). MS (ES+) m/z 230 (MH⁺, 100%), 252 (MNa+, 30%).

### b) 10-(methyloxy)-5-oxo-2,3-dihydro-1H,5H-pyrido[3,2,1-ij]quinoline-1-carbaldehyde

10-(methyloxy)-2,3-dihydro-1H,5H-pyrido[3,2,1-ij]quinoline-1,5-dione (0.092 g, 0.4 mmol) was converted to the enol ether by the general method of Example 2f). After overnight reaction the enol ether formation had not gone to completion, and the reaction mixture was added to another 5 equivalents of phosphorane prepared from methoxymethylenetriphenylphosphonium chloride (2.0 mmol) and potassium tert-butoxide (2.0 mmol), and stirred for a further 24 hrs. After this time, the reaction mixture was worked up as generally described for Example 2f). Chromatography of the crude product on silica gel eluting with 1-100% ethyl acetate in hexane afforded the desired enol ether in approx. 1:1 ratio with triphenylphosphine oxide (0.012 g). This material was treated with sodium iodide (0.076 g, 2 equiv) and trimethyl chlorosilane (0.062 mL, 2 equiv) by the general method of Example 2g). After work-up and chromatography on silica gel eluting with ethyl acetate/hexane mixtures, a sample of the title compound was obtained as a colourless oil (0.01 g).

¹H NMR δ(CDCl₃) 1.90-1.98 (1H, m), 2.62-2.68 (1H, m), 3.44-3.51, 3.96 (3H, s), 4.11-4.14 (1H, m), 4.68-4.74 (1H, m), 6.54 (1H, d, J 9.2 Hz), 6.90 (1H, d, J 8.4 Hz), 7.50 (1H, d, J 8.4 Hz), 7.61 (1H, d, J 9.2 Hz), 9.71 (1H, s). MS (ES+) m/z 244 (MH⁺, 100%).

### c) Title Compound

10-(methyloxy)-5-oxo-2,3-dihydro-1H,5H-pyrido[3,2,1-ij]quinoline-1-carbaldehyde (0.01 g, 0.04 mmol) was reacted with 1,1-dimethylethyl (2,3-dihydro[1,4]dioxino[2,3-c]pyridin-7-ylmethyl)4-piperidinylcarbamate (0.0016 g) (for a synthesis see WO2004058144 Example 99(h)) followed by deprotection with trifluoroacetic acid and chromatography as described in Example 1f). The free base of the title compound was obtained as a colourless oil (0.003 g,)

¹H NMR δ(CDCl₃) 1.47-1.57 (2H, m), 1.68-2.05 (m, contains 4H and water), 2.19-2.28 (1H, m), 2.38-2.40 (2H, m), 2.50-2.63 (2H, m), 2.73-2.78 (1H, m), 3.15 (1H, d, J 11.7 Hz), 3.42-3.55 (2H, m), 3.91 (3H, s), 4.26-4.35 (4H, m), 4.71-4.78 (1H, m), 6.54 (1H, d, J 9.5 Hz), 6.80-6.84 (2H, m), 7.40 (1H, d, J 8.5 Hz), 7.58 (1H, d, J 9.5 Hz), 8.10 (1H, s).

MS (ES+) m/z 477 (MH⁺, 100%).

The free base of the title compound was dissolved in dichloromethane (0.5 mL), treated with 1M HCl in ether (0.1 mL) and the solution stirred for 10 min. The solution was evaporated to dryness to give the title dihydrochloride as a pale yellow solid (0.0035 g).

### Example 13

1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one hydrochloride (diastereomer 1),

### Example 14

1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one dihydrochloride (diastereomer 1) and

### Example 15

1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one hydrochloride (diastereomer 2)

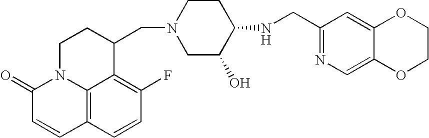

### a) Phenylmethyl (3R,4S)-4-amino-3-hydroxy-1-piperidinecarboxylate

cis-4-tert-Butoxycarbonylamino-3-hydroxy-piperidine-1-carboxylic acid benzyl ester Enantiomer 1 (for a synthesis see WO2004058144, Example 5(c)) (10.0 gm, 28.5 mmol) was dissolved in dry dichloromethane (50 ml) and trifluoroacetic acid (25 ml) added dropwise, then the reaction was stirred for 1 h under argon. The solvent was removed under reduced pressure, and the residue partioned between 10% methanol in dichloromethane and water. Potassium carbonate was added to this solution until the system became saturated. The phases were separated and the aqueous phase extracted with 10% methanol in dichloromethane. The combined organic phases were dried over anhydrous magnesium sulphate and evaporated under reduced pressure, to give the product as a white solid. (5.98 g, 84%)

¹H NMR δ(CDCl₃,) 1.54-1.62 (1H, m), 1.67-1.73 (1H, m), 1.75-1.95 (3H, m), 2.93-2.97 (2H, m), 3.06-3.10 (1H, m), 3.67 (1H, s), 3.98-4.10 (2H, m), 5.16 (1H, s), 7.32-7.40 (5H, m).

### b) Phenylmethyl (3R,4S)-4-[(2,3-dihydro[1,4]dioxino[2,3-c]pyridin-7-ylmethyl)amino]-3-hydroxy-1-piperidinecarboxylate

Phenylmethyl (3R,4S)-4-amino-3-hydroxy-1-piperidinecarboxylate (5.75 g, 23 mmol) and 2,3-dihydro[1,4]dioxino[2,3-c]pyridine-7-carboxaldehyde (3.71 g, 23 mmol) (for a synthesis see WO2004058144 Example 2(c) or WO2003087098 Example 19(d)) were dissolved in methanol/chloroform 1:1 (200 ml). Molecular sieves were added and the reaction heated to 65° C. for 4 h, then cooled to room temperature. Sodium triacetoxyborohydride (9.75 g, 46 mmol) was added and the reaction stirred for 15 min. The reaction mixture was filtered through kieselguhr and the solid washed with methanol. The filtrate was concentrated under reduced pressure and the residue partitioned between saturated NaHCO₃ and 20% methanol in dichloromethane. The aqueous phase was extracted with 20% methanol in dichloromethane. The organic phases were combined and dried over anhydrous magnesium sulphate and concentrated under reduced pressure. The residue was chromatographed on silica gel eluting with a gradient of 0-5% methanol in dichloromethane. Product-containing fractions were combined and evaporated to give the desired product as a solid (4.3 g, 46%).

MS (ES+) m/z 400 (MH+, 100%).

### c) Phenylmethyl (3R,4S)-4-((2,3-dihydro[1,4]dioxino[2,3-c]pyridin-7-ylmethyl){[(1,1-dimethylethyl)oxy]carbonyl}amino)-3-hydroxy-1-piperidinecarboxylate

Phenylmethyl (3R,4S)-4-[(2,3-dihydro[1,4]dioxino[2,3-c]pyridin-7-ylmethyl)amino]-3-hydroxy-1-piperidinecarboxylate (4.3 g, 11 mmol) was dissolved in methanol. Sodium hydrogen carbonate (2.8 g, 33 mmol) was added and the reaction stirred in an ice-bath at 0° C. for 30 min. After this time the reaction mixture was allowed to warm to 5-10° C. and di-tert-butyl dicarbonate (2.64 g, 12.1 mmol) added portionwise. After addition, the reaction mixture was stirred overnight at room temperature. The reaction mixture was filtered, the filtrate concentrated under reduced pressure, and chromatographed on silica gel eluting with 0-75% ethyl acetate in hexane. Product-containing fractions were combined and evaporated to give the title compound as a solid (3.5 g, 64%).

MS (ES+) 500 m/z (MH+, 100%).

### d) 1,1-dimethylethyl (2,3-dihydro[1,4]dioxino[2,3-c]pyridin-7-ylmethyl)[(3R,4S)-3-hydroxy-4-piperidinyl]carbamate

Phenylmethyl(3R,4S)-4-((2,3-dihydro[1,4]dioxino[2,3-c]pyridin-7-ylmethyl){[(1,1-dimethylethyl)oxy]carbonyl}amino)-3-hydroxy-1-piperidinecarboxylate (3.5 g, 7.0 mmol) was dissolved in ethanol (60 ml) and 10% Pd/C (paste) (2.85 g) was added. The reaction mixture was hydrogenated at atmospheric pressure overnight. The reaction mixture was filtered through kieselguhr, and washed with ethanol. The filtrate was concentrated under reduced pressure to give a white solid which was chromatographed on silica gel eluting with a gradient of 0-10% 2M methanolic ammonia in DCM. Product-containing fractions were combined and evaporated to give the desired compound as a solid (2.2 g, 86%).

MS (ES+) 366 m/z (MH+, 50%).

### e) (1R,S)-1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one

10-Fluoro-5-oxo-2,3-dihydro-1H,5H-pyrido[3,2,1-ij]quinoline-1-carbaldehyde (for a preparation see Example 2g)) (0.60 g 2.6 mmol) was dissolved in methanol (165 mL), treated with acetic acid (5 drops), followed by 1,1-dimethylethyl (2,3-dihydro[1,4]dioxino[2,3-c]pyridin-7ylmethyl)[(3R,4S)-3-hydroxy-4-piperidinyl]carbamate (0.95 g, 2.6 mmol). After 10 min the reaction was treated with (polystyrylmethyl)trimethylammonium cyanoborohydride (4.1 mmol/g, 5.1 g), and the mixture stirred at room temperature overnight. The reaction was filtered and evaporated to an oil. This was dissolved in dichloromethane (50 ml) and TFA (9.8 ml) and stirred at room temperature for 1 h. The solvent was evaporated and the residue chromatographed on silica gel eluting with a gradient of 0-10% methanol in dichloromethane. Product-containing fractions were combined and evaporated to give the product as a white solid (0.6 g, 55%).

¹H NMR δ(250 MHz CDCl₃,) 1.73-1.90 (3H, m), 2.21-2.34 (3H, m), 2.42-2.62 (5H, m), 2.74-2.83 (1H, m), 3.11-3.22 (1H, m), 3.43-3.63 (2H, m), 3.86 (2H, s), 3.89 (1H, s), 4.24-4.44 (4H, m), 4.73-4.80 (1H, m), 6.62-6.60 (1H, d, J, 9.5 Hz), 6.86-6.96 (2H, m), 7.37 7.41 (1H, m). 7.61-7.65 (1H, d, J, 9.5 Hz) 8.10 (1H, s

MS (ES+) m/z 481 (MH⁺, 100%).

### f) Title Compounds

(1RS)-1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one (0.047 g) was chromatographed on a Chiralpak AD-H 21×250 mm column, eluting with a mixture of 40:40:20:0.1 acetonitrile:methanol:isopropanol:isopropylamine at 13 ml/min. The peak with retention time of 19.9 min was collected and evaporated to give the free base of title Diastereomer 1 (0.0106 g) and the peak with retention time 28.6 min collected and evaporated to give the free base of title Diastereomer 2 (0.0056 g) The free bases were each dissolved in methanol and treated with 1.0 equiv. of aqueous 6M HCl. After stirring at room temperature for 1 hr, the solvent was removed under reduced pressure and the solids dried under reduced pressure at 40° C. to give the title compounds as the corresponding title mono-hydrochloride salts, each with a diastereomeric purity of >99%.

To form the dihydrochloride salt of Diastereomer 1 (Example 14), the free base of the title compound from Example 13 (Diastereomer 1) was slurried in methanol (5 mL) and treated with 2.0 equiv. of aqueous 6M HCl. The solvent was removed under reduced pressure and the solids dried under reduced pressure at 40° C.

### Example 16

### 1-({(3R,4S)-4-[(2,3-dihydro[1,4]dioxino[2,3-c]pyridin-7-ylmethyl)amino]-3-hydroxy-1-piperidinyl}methyl)-8-fluoro-2,3-dihydro-1H,5H-pyrido[3,2,1-ij]quinolin-5-one hydrochloride

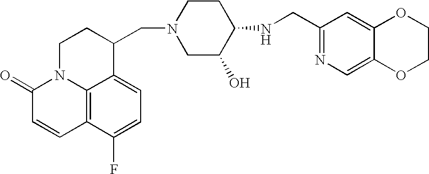

### a) Phenylmethyl (3R,4S)-4-[(2,3-dihydro[1,4]dioxino[2,3-c]pyridin-7-ylmethyl)amino]-3-hydroxy-1-piperidinecarboxylate

cis-4-tert-Butoxycarbonylamino-3-hydroxy-piperidine-1-carboxylic acid benzyl ester enantiomer 1 (for a synthesis see WO2004058144 Example 5(b)) (10.0 g, 28.5 mmol) was dissolved in dry dichloromethane (50 ml) and treated dropwise with TFA (25 ml). After stirring under argon for 1 h, the excess TFA was removed in vacuo and the residue partitioned between 10% methanol in DCM and water. Solid potassium carbonate was added until the aqueous layer was saturated, and the aqueous phase then washed with 10% methanol in DCM (×3). The combined organic layers were dried and evaporated to a white solid. (5.98 g). This material (5.75 g, 23 mmol) was dissolved in chloroform (100 ml) and methanol (100 ml), treated with 2,3-dihydro[1,4]dioxino[2,3-c]pyridine-7-carboxaldehyde (0.94 g, 5.7 mmole) (for a synthesis see WO2004058144, Example 2(c) or WO2003087098 Example 19(d)) (3.71 g, 22.5 mmol). 3A sieves were added and the reaction heated to 65° C. for 4 h. The reaction was cooled and treated with sodium triacetoxy borohydride (9.75, 46 mmol). After 15 min, the reaction was filtered through Kieselguhr and the filtrate concentrated in vacuo. The residues was partitioned between saturated sodium bicarbonate and 20% methanol in DCM. The aqueous layer was extracted with 20% methanol in DCM and the combined organic layers dried and evaporated. Chromatography of the residue on silica gel eluting with 0-5% methanol in DCM gave the title product (4.3 g, 46%)

MS (ES+) m/z 400 (MH⁺, 100%).

### b) Phenylmethyl (3R,4S)-4-((2,3-dihydro[1,4]dioxino[2,3-c]pyridin-7-ylmethyl){[(1,1-dimethylethyl)oxy]carbonyl}amino)-3-hydroxy-1-piperidinecarboxylate

Phenylmethyl (3R,4S)-4-[(2,3-dihydro[1,4]dioxino[2,3-c]pyridin-7-ylmethyl)amino]-3-hydroxy-1-piperidinecarboxylate (4.3 g, 11 mmol) was dissolved in methanol (80 ml) and treated with sodium bicarbonate (2.8 g, 33 mmol) at 0° C. After 30 min the reaction was allowed to warm to 5-10° C. and treated portionwise with di-tert-butyl dicarbonate (2.64 g, 12.1 mmol), and the reaction mixture was stirred at room temperature overnight under an argon atmosphere. The mixture was filtered, the filtrate evaporated and the residue chromatographed on silica gel eluring with 75%-100% ethyl acetate in hexane to give the title compound as a solid (3.5 g, 64%).

MS (ES+) m/z 500 (MH⁺, 100%).

### c) 1,1-dimethylethyl(2,3-dihydro[1,4]dioxino[2,3-c]pyridin-7-ylmethyl)[(3R,4S)-3-hydroxy-4-piperidinyl]carbamate

Phenylmethyl (3R,4S)-4-((2,3-dihydro[1,4]dioxino[2,3-c]pyridin-7-ylmethyl){[(1,1-dimethylethyl)oxy]carbonyl}amino)-3-hydroxy-1-piperidinecarboxylate (3.5 g, 7.0 mmol) was dissolved in ethanol (60 ml) and hydrogenated at atmospheric pressure with 10% Pd/C (2.85 g) overnight. The reaction mixture was filtered through Kieselguhr, the solid washed with ethanol and the filtrate concentrated in vacuo. The residue was chromatographed on silica gel eluring with 1-10% 2M methanolic ammonia in DCM to give the title compound as a solid (2.2 g, 86%).

MS (ES+) m/z 366 (MH⁺, 50%), 388 (MNa⁺, 20%).

### d) Title Compound

The title compound was prepared by the general method of Example 1f) from 8-fluoro-5-oxo-2,3-dihydro-1H,5H-pyrido[3,2,1-ij]quinoline-1-carbaldehyde (for a preparation see Example 5d)) (0.08 g, 0.35 mmole) and 1,1-dimethylethyl (2,3-dihydro[1,4]dioxino[2,3-c]pyridin-7-ylmethyl)[(3R,4S)-3-hydroxy-4-piperidinyl]carbamate (0.126 g, 0.35 mmole) to give the free base of the title compound (0.073 g, 44%).

¹H NMR δ(CDCl₃) 1.68-1.81 (2H, m), 1.93-2.03 (1H, m), 2.05-2.12 (1H, m), 2.15-2.65 (7H, m), 2.75-2.95 (2H, m), 2.97-3.02 (1H, m), 3.05-3.17 (1H, m), 3.68-3.78 (1H, m), 3.82 (2H, s), 4.24-4.37 (4H, m), 4.45-4.55 (1H, m), 6.72 (1H, d, J 6 Hz), 6.81-6.87 (2H, m), 7.29-7.40 (1H, m), 7.93 (1H, d, J 6 Hz), 8.11 (1H, s).

MS (ES+) m/z 481 (MH⁺, 40%).

The free base of the title compound was dissolved in dichloromethane (7 mL) and treated with 1.2 equivalents of 1M hydrogen chloride in diethyl ether then evaporated to dryness. Trituration with diethyl ether and filtration to give the title compound as an off-white solid.

### Example 17

### 10-fluoro-1-[((3R,4S)-3-hydroxy-4-{[(3-oxo-3,4-dihydro-2H-pyrido[3,2-b][1,4]oxazin-6-yl)methyl]amino}-1-piperidinyl)methyl]-2,3-dihydro-1H,5H-pyrido[3,2,1-ij]quinolin-5-one dihydrochloride

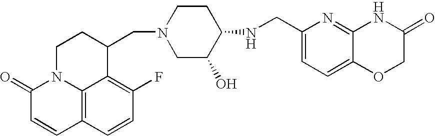

### a) 1,1-Dimethylethyl{(3R,4S)-1-[(10-fluoro-5-oxo-2,3-dihydro-1H,5H-pyrido[3,2,1-ij]quinolin-1-yl)methyl]-3-hydroxy-4-piperidinyl}carbamate

cis-(3-Hydroxy-piperidin-4-yl)-carbamic acid tert-butyl ester Enantiomer 1 (WO 2004058144 Example 5(c) (0.402 g, 2.0 mmol) and 10-Fluoro-5-oxo-2,3-dihydro-1H,5H-pyrido[3,2,1-ij]quinoline-1-carbaldehyde (for a preparation see Example 2g)) (0.460 g, 2.0 mmol) were dissolved in methanol (80 ml) and treated with acetic acid (0.8 ml). (Polystyrylmethyl)trimethylammonium cyanoborohydride (4.1 mmol/g, 2.0 g, 8.4 mmol) was added, and the mixture stirred at room temperature for 18 h. The reaction mixture was decanted from the supported reagent, evaporated, and the residue chromatographed on silica gel eluting with a gradient of 0-10% 2M methanolic ammonia in dichloromethane. Product-containing fractions were combined and evaporated to give the desired product as a pale yellow foam (668 mg, 77%).

MS (ES+) m/z 432 (MH+, 100%).

### b) 1-{[(3R,4S)-4-Amino-3-hydroxy-1-piperidinyl]methyl}-10-fluoro-2,3-dihydro-1H,5H-pyrido[3,2,1-ij]quinolin-5-one

1,1-Dimethylethyl{(3R,4S)-1-[(10-fluoro-5-oxo-2,3-dihydro-1H,5H-pyrido[3,2,1-ij]quinolin-1-yl)methyl]-3-hydroxy-4-piperidinyl}carbamate (668 mg, 1.55 mmol) was dissolved in dichloromethane (20 ml) and treated with trifluoroacetic acid (10 ml). The solvent was evaporated and the residue chromatographed on silica gel eluting with a gradient of 0-20% 2M methanolic ammonia in dichloromethane. Product-containing fractions were combined and evaporated to give the desired product as a colourless oil (0.330 g, 64%).

MS (ES+) m/z 332 (MH+, 100%).

### c) Title Compound

1-{[(3R,4S)-4-amino-3-hydroxy-1-piperidinyl]methyl}-10-fluoro-2,3-dihydro-1H,5H-pyrido[3,2,1-ij]quinolin-5-one (0.2 mmol, 0.066 g) and 3-oxo-3,4-dihydro-2H-pyrido[3,2-b][1,4]oxazine-6-carboxaldehyde (for a synthesis see WO2003087098 Example 31(e)) (0.2 mmol, (0.033 g) were dissolved in methanol (5 ml) containing acetic acid (0.1 ml). (Polystyrylmethyl)trimethylammonium cyanoborohydride (4.1 mmol/g, 0.2 g, 0.84 mmol) was added, and the mixture stirred at room temperature for 18 h. The reaction mixture was filtered, the filtrate evaporated, and the residue chromatographed on silica gel eluting with a gradient of 0-10% 2M methanolic ammonia in dichloromethane. Product-containing fractions were combined and evaporated to give the free base of the title compound as a pale yellow foam (0.075 g, 76%).

¹H NMR δ(CDCl₃) 1.80-1.86 (3H, m), 2.02-2.08 (1H, m), 2.20-2.23 (1H, m), 2.41-2.66 (4H, m), 2.76-3.18 (2H, m), 3.46-3.54 (2H, m), 3.86-3.95 (3H, m), 4.60 (2H, s), 4.76 (1H, dd, J 4.0, 14.8 Hz), 6.64 (1H, d, J 9.6 Hz), 6.88-6.97 (2H, m), 7.20 (1H, dd, J 1.6, 8.0 Hz), 7.38-7.42 (1H, m) 7.63 (1H, d, J 9.6 Hz).

MS (ES+) m/z 494 (MH⁺, 100%).

The free base of the title compound was dissolved in methanol (3.0 ml), treated with 1M HCl in ether (4.0 equiv) and the solution stirred for 20 min. The solution was evaporated to dryness and triturated with diethyl ether to give the title dihydrochloride salt as a pale yellow solid.

### Example 18

### 10-fluoro-1-[((3R,4S)-3-hydroxy-4-{[(3-oxo-3,4-dihydro-2H-pyrido[3,2-b][1,4]thiazin-6-yl)methyl]amino}-1-piperidinyl)methyl]-2,3-dihydro-1H,5H-pyrido[3,2,1-ij]quinolin-5-one dihydrochloride

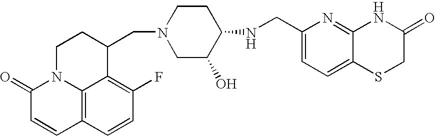

The title compound was prepared from 1-{[(3R,4S)-4-amino-3-hydroxy-1-piperidinyl]methyl}-10-fluoro-2,3-dihydro-1H,5H-pyrido[3,2,1-ij]quinolin-5-one (for a preparation see Example 17b)) (0.2 mmol, 0.066 g) and 3-oxo-3,4-dihydro-2H-pyrido[3,2-b][1,4]thiazine-6-carboxaldehyde (for a synthesis see WO2004058144 Example 7(d)) (0.2 mmol, (0.033 g) by the general method of Example 17c). The free base of the title compound was obtained as a pale yellow foam (0.078 g, 77%).

¹H NMR δ(CDCl₃) 1.80-1.86 (3H, m), 2.03-2.08 (1H, m), 2.10-2.23 (1H, m), 2.36-2.62 (4H, m), 2.78-3.17 (2H, m), 3.46 (2H, s), 3.47-3.54 (2H, m), 3.85-3.96 (3H, m), 4.75-4.79 (1H, m), 6.64 (1H, d, J 9.6 Hz), 6.92 (1H, dt, J 2.0, 8.8 Hz), 7.01 (1H, d, J 7.6 Hz), 7.40 (1H, dd, J 6.0, 8.8 Hz), 7.57-7.59 (1H, m) 7.63 (1H, d, J 9.6 Hz). MS (ES+) m/z 510 (MH⁺, 100%).

The free base of the title compound was dissolved in methanol (3.0 ml), treated with 1M HCl in ether (4.0 equiv) and the solution stirred for 20 min. The solution was evaporated to dryness and triturated with diethyl ether to give the title dihydrochloride salt as a pale yellow solid.

### Example 19

### 1-({(3R,4S)-4-[(6,7-dihydro[1,4]dioxino[2,3-c]pyridazin-3-ylmethyl)amino]-3-hydroxy-1-piperidinyl}methyl)-10-fluoro-2,3-dihydro-1H,5H-pyrido[3,2,1-ij]quinolin-5-one dihydrochloride

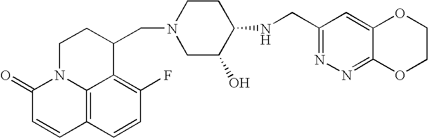

The title compound was prepared from 1-{[(3R,4S)-4-amino-3-hydroxy-1-piperidinyl]methyl}-10-fluoro-2,3-dihydro-1H,5H-pyrido[3,2,1-ij]quinolin-5-one (for a preparation see Example 17b)) (0.2 mmol, 0.066 g) and 6,7-dihydro[1,4]dioxino[2,3-c]pyridazine-3-carbaldehyde (for a preparation see Example 7g)) (0.2 mmol, (0.033 g) by the general method of Example 17c). The free base of the title compound was obtained as a pale yellow foam (0.072 g, 75%).

¹H NMR δ(CDCl₃) 1.73-1.88 (3H, m), 2.03-2.06 (1H, m), 2.18-2.22 (1H, m), 2.36-2.56 (4H, m), 2.76-3.14 (2H, m), 3.46-3.54 (2H, m), 3.89 (1H, br s), 4.00-4.08 (2H, m), 4.37-4.40 (2H, m), 4.51-4.54 (2H, m), 4.77 (1H, d, J 12.4 Hz), 6.64 (1H, d, J 9.6 Hz), 6.93 (1H, dt, J 2.0, 8.8 Hz), 7.01 (1H, s,), 7.40 (1H, dd, J 6.0, 8.8 Hz), 7.64 (1H, d, J 9.6 Hz).

MS (ES+) m/z 482 (MH⁺, 100%).

The free base of the title compound was dissolved in methanol (3.0 ml), treated with 1M HCl in ether (4.0 equiv) and the solution stirred for 20 min. The solution was evaporated to dryness and triturated with diethyl ether to give the title dihydrochloride salt as a pale yellow solid.

### Example 20

### 1-({(3R,4S)-4-[(6,7-dihydro[1,4]oxathiino [2,3-c]pyridazin-3-ylmethyl)amino]-3-hydroxy-1-piperidinyl}methyl)-10-fluoro-2,3-dihydro-1H,5H-pyrido[3,2,1-ij]quinolin-5-one dihydrochloride

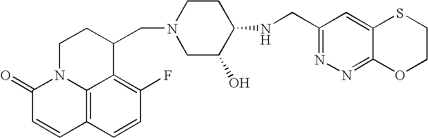

The title compound was prepared from 1-{[(3R,4S)-4-amino-3-hydroxy-1-piperidinyl]methyl}-10-fluoro-2,3-dihydro-1H,5H-pyrido[3,2,1-ij]quinolin-5-one (for a preparation see Example 17b)) (0.2 mmol, 0.066 g) and 6,7-dihydro[1,4]oxathiino[2,3-c]pyridazine-3-carbaldehyde (for a preparation see Example 9d) or WO2007081597 Example 17(d)) (0.2 mmol, (0.033 g) by the general method of Example 17c). The free base of the title compound was obtained as a pale yellow foam (0.051 g, 52%).

¹H NMR δ(CDCl₃) 1.63-1.88 (3H, m), 2.02-2.42 (4H, m), 2.45-2.57 (4H, m), 2.79 and 2.97 (1H, 2×d J 10.0 Hz), 2.98 and 3.13 (1H, 2×d, J 11 Hz), 3.20-3.32 (2H, m), 3.45-3.58 (3H, m), 3.89 (1H, br.s.), 3.93-4.06 (2H, m), 4.64-4.66 (2H, m), 4.77 (1H, d, J 12.4 Hz), 6.65 (1H, d, J 9.6 Hz), 6.93 (1H, t, J 8.8 Hz), 7.39-7.43 (2H, m), 7.64 (1H, d, J 9.6 Hz).

MS (ES+) m/z 498 (MH⁺, 100%).

The free base of the title compound was dissolved in methanol (3.0 ml), treated with 1M HCl in ether (4.0 equiv) and the solution stirred for 20 min. The solution was evaporated to dryness and triturated with diethyl ether to give the title dihydrochloride salt as a pale yellow solid.

### Example 21

### 10-fluoro-1-({(3R,4S)-3-hydroxy-4-[([1,3]oxathiolo[5,4-c]pyridin-6-ylmethyl)amino]-1-piperidinyl}methyl)-2,3-dihydro-1H,5H-pyrido[3,2,1-ij]quinolin-5-one dihydrochloride

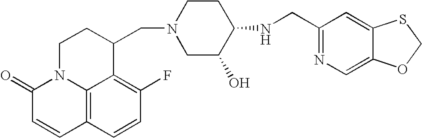

The title compound was prepared from 1-{[(3R,4S)-4-amino-3-hydroxy-1-piperidinyl]methyl}-10-fluoro-2,3-dihydro-1H,5H-pyrido[3,2,1-ij]quinolin-5-one (for a preparation see Example 17b)) (0.2 mmol, 0.066 g) and [1,3]oxathiolo[5,4-c]pyridine-6-carbaldehyde (for a synthesis see WO2004058144, Example 61) by the general method of Example 17c). The free base of the title compound was obtained as a pale yellow foam (0.067 g, 70%).

¹H NMR δ(CDCl₃) 1.63-1.85 (3H, m), 2.02-2.38 (2H, m), 2.42-2.60 (6H, m), 2.75 and 2.87 (1H, 2×d J 10.0 Hz), 2.98 and 3.13 (1H, 2×d, J 11 Hz), 3.46-3.53 (2H, m), 3.88 (3H, appears as br.s.), 4.77 (1H, d, J 14.8 Hz), 5.73 (2H, s), 6.64 (1H, d, J 9.6 Hz), 6.93 (1H, t, J 8.8 Hz), 7.24 (1H, d, J 2.0 Hz) 7.40 (1H, dd, J 6.0, 8.8 Hz), 7.63 (1H, d, J 9.6 Hz), 8.001H, s.)

MS (ES+) m/z 483 (MH⁺, 100%), 505(MNa+, 30%).

The free base of the title compound was dissolved in methanol (3.0 ml), treated with 1M HCl in ether (4.0 equiv) and the solution stirred for 20 min. The solution was evaporated to dryness and triturated with diethyl ether to give the title dihydrochloride salt as a pale yellow solid.

### Example 22

### 1-({4-[(2,3-dihydro[1,4]dioxino[2,3-c]pyridin-7-ylmethyl)amino]-1-piperidinyl}methyl)-10-fluoro-1-hydroxy-2,3-dihydro-1H,5H-pyrido[3,2,1-ij]quinolin-5-one hydrochloride

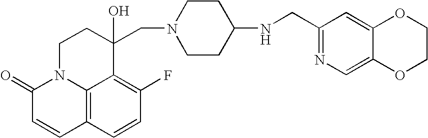

### a) 10-Fluoro-1-hydroxy-1-(hydroxymethyl)-2,3-dihydro-1H,5H-pyrido[3,2,1-ij]quinolin-5-one

To a mixture of trimethylsulfoxonium iodide (0.79 g, 3.6 mmole) and dimethylsulphoxide (8 mL) at RT was added 60% sodium hydride/oil (0.143 g, 3.6 mmole) and stirred for 30 min. 10-Fluoro-2,3-dihydro-1H,5H-pyrido[3,2,1-ij]quinoline-1,5-dione (for a preparation see Example 2e)) (0.6 g, 2.8 mmole) was added and the mixture stirred for a further 2 h. 2M Hydrochloric acid (10 mL) and water (10 mL) was added and the mixture extracted with ethyl acetate which gave an emulsion which was filtered through kieselguhr washing with more ethyl acetate. The combined organic phases were washed with more water then saturated brine and dried over sodium sulphate, filtered and evaporated to dryness. Chromatography on silica gel with a gradient of 0-15% methanol/dichloromethane gave the title compound (0.083 g, 12%).

MS (ES+) m/z 250 (MH⁺, 100%).

### b) (10-Fluoro-1-hydroxy-5-oxo-2,3-dihydro-1H,5H-pyrido[3,2,1-ij]quinolin-1-yl)methyl 4-methylbenzenesulfonate

10-Fluoro-1-hydroxy-1-(hydroxymethyl)-2,3-dihydro-1H,5H-pyrido[3,2,1-ij]quinolin-5-one (0.083 g, 0.33 mmole) was suspended in a mixture of dichloromethane (3 mL), THF (3 mL) and dimethylformamide (0.3 mL) and treated with triethylamine (0.07 mL, 0.5 mmole), 4-methylbenzenesulfonyl chloride (0.064 g, 0.33 mmole) and dibutyltin(IV) oxide and stirred at RT for 18 h. Water was added and the mixture separated into two layers. The organic phase was separated then washed with saturated sodium bicarbonate solution, dried over magnesium sulphate filtered and evaporated to dryness to give the title compound as an orange oil (0.107 g, 80%).

MS (ES+) m/z 404 (MH⁺, 100%).

### c) Title Compound

A mixture of (10-fluoro-1-hydroxy-5-oxo-2,3-dihydro-1H,5H-pyrido[3,2,1-ij]quinolin-1-yl)methyl 4-methylbenzenesulfonate (0.107 g, 0.26 mmole) ethanol (15 mL) and 1,1-dimethylethyl (2,3-dihydro[1,4]dioxino[2,3-c]pyridin-7-ylmethyl)4-piperidinylcarbamate (0.083 g, 0.24 mmole) (for a synthesis see WO2004058144 Example 99(h)) was treated with sodium carbonate (0.081 g, 0.77 mmole) and stirred at RT for 18 h then at 50° C. for 2 h. A further amount of 1,1-dimethylethyl (2,3-dihydro[1,4]dioxino[2,3-c]pyridin-7-ylmethyl)4-piperidinylcarbamate (0.028 g, 0.08 mmole) was added and heating continued for another 2 h. The mixture was evaporated to dryness and the residue partitioned between water and dichloromethane. The organic phase was dried over magnesium sulphate, filtered and evaporated to dryness to give the Boc protected compound which was dissolved in dichloromethane (15 mL) and treated with TFA (1 mL) and stirred at RT overnight. The mixture was then evaporated to dryness and the residue partitioned between saturated potassium carbonate in water and dichloromethane. The organic phase was separated and the combined extracts after 2 further extractions were dried over magnesium sulphate filtered and evaporated to dryness. Chromatography on silica gel eluting with a gradient of 0-15% methanol/dichloromethane gave the free base of the title compound (0.054 g, 54%).

¹H NMR δ(CDCl₃) 1.45-1.91 (6H, m), 2.12-2.18 (1H, m), 2.20-2.27 (1H, m), 2.3-2.45 (2H, m), 2.47-2.53 (1H, m), 2.7 (1H, d, J 14 Hz), 2.75-2.95 (3H, m), 3.7-3.77 (1H, m), 3.8 (2H, s), 4.22-4.35 (4H, m), 4.59-4.67 (1H, m), 6.62 (1H, d, J 9.6 Hz), 6.81 (1H, s), 6.89-6.95 (1H, m), 7.39-7.45 (1H, m), 7.62 (1H, d, J 9.6 Hz), 8.1 (1H, s).

MS (ES+) m/z 481 (MH⁺, 40%).

The free base of the title compound was dissolved in dichloromethane (5 mL) and treated with 1.2 equivalents of 1M hydrogen chloride in diethyl ether then evaporated to dryness to give the title compound

### Example 23

### 1-({4-[(2,3-dihydro[1,4]dioxino[2,3-c]pyridin-7-ylmethyl)amino]-1-piperidinyl}methyl)-8-fluoro-1-hydroxy-2,3-dihydro-1H,5H-pyrido[3,2,1-ij]quinolin-5-one hydrochloride

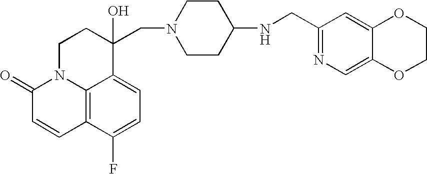

### a) 8-Fluoro-1-methylidene-2,3-dihydro-1H,5H-pyrido[3,2,1-ij]quinolin-5-one

To a solution of methyltriphenylphosphonium bromide (2.1 g, 6 mmole) in dry THF (25 mL) was added 2.5M n-butyllithium in hexane (2.4 mL) and stirred at RT under argon for 2 h. 8-Fluoro-2,3-dihydro-1H,5H-pyrido[3,2,1-ij]quinoline-1,5-dione (for a preparation see Example 5b)) (1.09 g, 5 mmole) was then added and the mixture stirred at RT for 2 days. The mixture was then evaporated to dryness and the residue partitioned between ethyl acetate and water. The organic phase was separated and washed with saturated brine then dried over magnesium sulphate, filtered and evaporated to dryness. Chromatography on silica gel eluting with 0-100% ethyl acetate/hexane gave the title compound as a colourless oil (0.08 g).

MS (ES+) m/z 216 (MH+, 100%).

### b) 8-Fluoro-1-hydroxy-1-(hydroxymethyl)-2,3-dihydro-1H,5H-pyrido[3,2,1-ij]quinolin-5-one

8-Fluoro-1-methylidene-2,3-dihydro-1H,5H-pyrido[3,2,1-ij]quinolin-5-one (0.08 g, 0.37 mmole) was dissolved in t-butylalcohol (1 mL) and water (1 mL) and treated with a 1:1 mixture of AD-mix-α and AD-mix-β (0.39 g) and stirred at RT for 2 h. The mixture was then partitioned between 20% methanol in dichloromethane and water. The organic phase was separated and the aqueous phase re-extracted. The combined organic phases were dried over magnesium sulphate filtered and evaporated to give the title compound as an off-white solid (0.052 g).

MS (ES+) m/z 272(MNa+, 20%), 250 (MH+, 100).

### c) (8-Fluoro-1-hydroxy-5-oxo-2,3-dihydro-1H,5H-pyrido[3,2,1-ij]quinolin-1-yl)methyl 4-methylbenzenesulfonate

The title compound was prepared by the general method of Example 22b) from 8-fluoro-1-hydroxy-1-(hydroxymethyl)-2,3-dihydro-1H,5H-pyrido[3,2,1-ij]quinolin-5-one (0.062 g, 0.25 mmole) to give (0.083 g).

MS (ES+) m/z 404 (MH⁺, 100%).

### d) Title Compound

The title compound was prepared by the general method of Example 22c) from 8-fluoro-1-hydroxy-5-oxo-2,3-dihydro-1H,5H-pyrido[3,2,1-ij]quinolin-1-yl)methyl 4-methylbenzenesulfonate (0.083 g, 0.21 mmole) and 1,1-dimethylethyl (2,3-dihydro[1,4]dioxino[2,3-c]pyridin-7-ylmethyl)4-piperidinylcarbamate (0.059 g, 0.17 mmole) (for a synthesis see WO2004058144 Example 99(h)) to give the free base as a white solid (0.032 g).

¹H NMR δ(CDCl₃) 1.45-1.55 (2H, m), 1.61-1.95 (4H, m), 2.07-2.13 (1H, m), 2.18-2.23 (1H, m), 2.31-2.49 (2H, m), 2.51-2.58 (2H, m), 2.71 (1H, d, J 14 Hz), 2.73-2.79 (1H, m), 2.84-2.91 (1H, m), 3.71-3.79 (1H, m), 3.81 (2H, s), 4.25-4.38 (4H, m), 4.58-4.63 (1H, m), 6.71 (1H, d, J 9.6 Hz), 6.81 (1H, s), 6.86-6.91 (1H, m), 7.65-7.69 (1H, m), 7.95 (1H, d, J 9.6 Hz), 8.09 (1H, s).

MS (ES+) m/z 481 (MH⁺, 35%).

The free base of the title compound was dissolved in dichloromethane (5 mL) and treated with 1.2 equivalents of 1M hydrogen chloride in diethyl ether then evaporated to dryness to give the title compound

### Example 24

### 1-[(2-{[(2,3-Dihydro [1,4]dioxino[2,3-c]pyridin-7-ylmethyl)amino]methyl}-4-morpholinyl)methyl]-10-fluoro-2,3-dihydro-1H,5H-pyrido[3,2,1-ij]quinolin-5-one hydrochloride

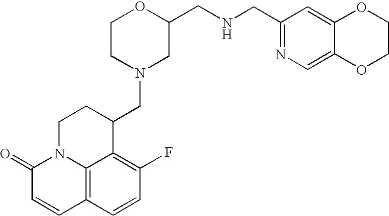

### a) 1-(2,3-Dihydro [1,4]dioxino[2,3-c]pyridin-7-yl)-N-{[4-(phenylmethyl)-2-morpholinyl]methyl}methanamine

2,3-Dihydro[1,4]dioxino[2,3-c]pyridine-7-carbaldehyde (0.8 g, 4.85 mmole) (for a synthesis see WO2004058144, Example 2(c) or WO2003087098 Example 19(d)) and 1-[4-(phenylmethyl)-2-morpholinyl]methanamine (1 g, 4.85 mmole) were dissolved in methanol (50 mL) and acetic acid (2 drops) was added along with (polystyrylmethyl)trimethylammonium cyanoborohydride (4.1 mmole/g, 4.74 g, 19.4 mmole). The mixture was stirred at RT for 18 h then filtered and the filtrate evaporated to dryness. The resulting residue was chromatographed on silica gel eluting with a gradient of 7-20% methanol/dichloromethane to give the title compound (1.49 g).

MS (ES+) m/z 356 (MH⁺, 50%).

### b) 1,1-Dimethylethyl (2,3-dihydro[1,4]dioxino[2,3-c]pyridin-7-ylmethyl){[4-(phenylmethyl)-2-morpholinyl]methyl}carbamate

1-(2,3-Dihydro [1,4]dioxino[2,3-c]pyridin-7-yl)-N-{[4-(phenylmethyl)-2-morpholinyl]methyl}methanamine (1.49 g, mmole) was dissolved in dichloromethane (100 mL) and treated with di-tert-butyl dicarbonate (0.92 g, 4.2 mmole) and stirred at RT for 18 h. The mixture was evaporated to dryness and chromatographed on silica gel eluting with a gradient of 0-10% methanol/dichloromethane then re chromatographed on more silica gel eluting with a gradient of 50-100% ethyl acetate/petroleum ether to give the title compound (0.611 g).

MS (ES+) m/z 456 (MH⁺, 25%).

### c) 1,1-Dimethylethyl (2,3-dihydro[1,4]dioxino[2,3-c]pyridin-7-ylmethyl)(2-morpholinylmethyl)carbamate

1,1-Dimethylethyl (2,3-dihydro[1,4]dioxino[2,3-c]pyridin-7-ylmethyl){[4-(phenylmethyl)-2-morpholinyl]methyl}carbamate (0.61 g, 1.3 mmole) was hydrogenated at S.T.P in ethanol (80 mL) over a 20% palladium hydroxide on carbon catalyst for 18 h. The catalyst was removed by filtration and the filtrate evaporated to dryness to give the title compound (0.614 g).

MS (ES+) m/z 366 (MH⁺, 15%).

### d) Title Compound

10-Fluoro-5-oxo-2,3-dihydro-1H,5H-pyrido[3,2,1-ij]quinoline-1-carbaldehyde (for a preparation see Example 2g)) (0.233 g, 1 mmole) and 1,1-dimethylethyl (2,3-dihydro[1,4]dioxino[2,3-c]pyridin-7-ylmethyl)(2-morpholinylmethyl)carbamate (0.614 g, 1.68 mmole) were dissolved in methanol (70 mL), treated with acetic acid (2 drops) and (polystyrylmethyl)trimethylammonium cyanoborohydride (Novabiochem) (4.1 mmole/g, 1.0 g, 4.1 mmole), and the mixture stirred at RT for 60 h. The mixture was then filtered and the resin washed with DCM and the filtrate evaporated to dryness to give the Boc protected intermediate which was used without further purification. Deprotection with TFA (5.2 mL) in dichloromethane (40 mL) at RT overnight. The mixture was then evaporated to dryness and the residue partitioned between saturated potassium carbonate in water and 10% methanol/dichloromethane. The combined organic phases were separated and dried over magnesium sulphate filtered and evaporated to dryness. Chromatography on silica gel eluting with a gradient of 0-20% methanol/dichloromethane gave the free base of the title compound (0.34 g).

¹H NMR δ(CDCl₃) 1.72-1.83 (1H, m), 1.89-1.95 (1H, m), 2.05-2.22 (1H, m), 2.29-2.75 (5H, m), 2.86-2.95 (1H, m), 3.43-3.54 (2H, m), 3.63-3.90 (6H, m), 4.23-4.34 (4H, m), 4.72-4.79 (1H, m), 6.61 (1H, d, J 9.6 Hz), 6.79-6.83 (1H, m), 6.87-6.93 (1H, m), 7.39-7.44 (1H, m), 7.62 (1H, d, J 9.6 Hz), 8.08-8.11 (1H, m).

MS (ES+) m/z 481 (MH⁺, 60%).

The free base of the title compound was dissolved in dichloromethane (5 mL) and treated with 1.2 equivalents of 1M hydrogen chloride in diethyl ether then evaporated to dryness to give the title compound

### Example 25

### 7-Chloro-6-({4-[(2,3-dihydro[1,4]dioxino[2,3-c]pyridin-7-ylmethyl)amino]-1-piperidinyl}methyl)-5,6-dihydro-4H-[1,3]thiazolo[5,4,3-ij]quinolin-2-one dihydrochloride

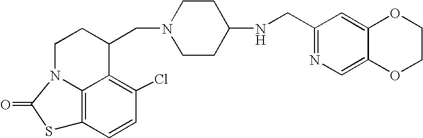

### a) Ethyl 3-(5-chloro-2-oxo-1,3-benzothiazol-3(2H)-yl)propanoate

5-Chloro-1,3-benzothiazol-2(3H)-one (2.0 g, 11 mmole) in toluene (100 mL) was treated with ethyl acrylate (1.3 mL, 12 mmole) tetraethyl orthosilicate (2.5 mL, 11 mmole) and cesium fluoride (0.18 g, 1.2 mmole) and heated under reflux for 18 h. The mixture was allowed to cool then partitioned between ethyl acetate (2×100 mL) and water (100 mL). The organic layer was separated, dried over sodium sulphate, filtered and evaporated to dryness. Chromatography on silica gel eluting with a gradient of 10-50% ethyl acetate/40-60 petroleum ether gave the title compound (2.24 g, 73%).

MS (ES+) m/z 308 and 310 (M+Na, 40 and 14%).

### b) 3-(5-Chloro-2-oxo-1,3-benzothiazol-3(2H)-yl)propanoic acid

Ethyl 3-(5-chloro-2-oxo-1,3-benzothiazol-3(2H)-yl)propanoate (2.24 g, 7.8 mmole) in ethanol (50 ml) was treated with 2N sodium hydroxide solution (8 mL) and stirred at RT for 2 h. The ethanol was then removed in-vacuo and water (20 mL) added. The mixture was then acidified with 5N hydrochloric acid. The resulting precipitate was filtered off and dried in-vacuo to give the title compound (1.9 g, 94%).

MS (ES+) m/z 270 and 272 (M+Na, 80 and 25%).

### c) 7-Chloro-4H-[1,3]thiazolo[5,4,3-ij]quinoline-2,6(5H)-dione

3-(5-Chloro-2-oxo-1,3-benzothiazol-3(2H)-yl)propanoic acid (0.5 g, 1.9 mmole) in dichloromethane (50 mL) was treated with oxalyl chloride (0.36 mL, 4 mmole) and DMF (2 drops) and stirred at RT for 2 h. The mixture was then evaporated to dryness and re-dissolved in dichloromethane (50 mL). Aluminium chloride (0.31 g, 2.3 mmole) was then added and the mixture stirred at RT for 2 h. A further amount of aluminium chloride (0.31 g, 2.3 mmole) was added and the mixture stirred at RT for 2 days. Water (50 mL) was added and the mixture extracted with 10% methanol/dichloromethane (2×30 mL). The combined organic phases were dried over sodium sulphate, filtered and evaporated to dryness. Chromatography on silica gel eluting with a gradient of 0-5% methanol/dichloromethane gave the title compound (0.42 g, 90%).

MS (ES+) m/z 240 and 242 (MH⁺, 100 and 30%).

### d) 7-Chloro-6-[(methyloxy)methylidene]-5,6-dihydro-4H-[1,3]thiazolo[5,4,3-ij]quinolin-2-one

Triphenyl(methoxymethyl)phosphonium chloride (1.8 g, 5.2 mmole) in 1,4-dioxane (50 mL) was treated with potassium tert-butoxide (0.59 g, 5.2 mmole) and stirred at RT for 30 mins. A solution of 7-chloro-4H-[1,3]thiazolo[5,4,3-ij]quinoline-2,6(5H)-dione (0.42 g, 1.7 mmole) in 1,4-dioxane (20 mL) was then added and the mixture stirred at RT for a further 18 h. The mixture was then partitioned between water (150 mL) and ethyl acetate (2×200 mL) and the combined organics dried over sodium sulphate, filtered and evaporated to dryness. Chromatography on silica gel eluting with a gradient of 5-50% ethyl acetate/40-60 petroleum ether gave the title compound (0.42 g, 89%).

MS (ES+) m/z 268 and 270 (MH⁺, 100 and 30%).

### e) 7-Chloro-2-oxo-5,6-dihydro-4H-[1,3]thiazolo[5,4,3-ij]quinoline-6-carbaldehyde

7-Chloro-6-[(methyloxy)methylidene]-5,6-dihydro-4H-[1,3]thiazolo[5,4,3-ij]quinolin-2-one (0.42 g, 1.6 mmole) in dry acetonitrile (20 mL) was treated with chlorotrimethylsilane (0.3 mL, 2.4 mmole) and sodium iodide (0.353 g, 2.4 mmole) and stirred at 40° C. for 18 h. The mixture was then evaporated to dryness and the residue partitioned between water and ethyl acetate. The organic phase was then washed with a solution of sodium metabisulphite and then saturated brine then separated and dried over sodium sulphate, filtered and evaporated to dryness. Chromatography on silica gel eluting with a gradient of 5-40% ethyl acetate/40-60 petroleum ether gave the title compound (0.26 g, 65%).

MS (ES+) m/z 254 and 256 (MH⁺, 100 and 30%).

### f) 6-[(4-Amino-1-piperidinyl)methyl]-7-chloro-5,6-dihydro-4H-[1,3]thiazolo[5,4,3-ij]quinolin-2-one

The title compound was prepared by the general method of Example 1f) from 7-chloro-2-oxo-5,6-dihydro-4H-[1,3]thiazolo[5,4,3-ij]quinoline-6-carbaldehyde (0.26 g, 1 mmole) and 4-(N-Boc-amino)piperidine (0.2 g, 1 mmol). After deprotection with TFA, the resulting TFA salt was passed through a Varian Mega Bond elut SAX cartridge in methanol to give the free base (0.17 g).

MS (ES+) m/z 338 and 340 (MH⁺, 100 and 30%).

### g) Title Compound

The title compound was prepared from 6-[(4-amino-1-piperidinyl)methyl]-7-chloro-5,6-dihydro-4H-[1,3]thiazolo[5,4,3-ij]quinolin-2-one (0.085 g, 0.25 mmole) and 2,3-dihydro[1,4]dioxino[2,3-c]pyridine-7-carboxaldehyde (0.041 g, 0.25 mmole) (for a synthesis see WO2004058144, Example 2(c) or WO2003087098 Example 19(d)) by the general method of Example 17c) to give the free base (0.04 g).

¹H NMR δ(CDCl₃) 1.49-1.63 (2H, m), 1.68-1.81 (1H, m), 1.88-2.05 (3H, m), 2.19-2.31 (1H, m), 2.32-2.38 (1H, m), 2.45-2.52 (1H, m), 2.59-2.82 (3H, m), 3.11-3.18 (1H, m), 3.41-3.55 (2H, m), 3.89 (2H, s), 4.22-4.37 (5H, m), 6.82 (1H, s), 7.11 (1H, d, J 8.4 Hz), 7.21 (1H, d, J 8.4 Hz), 8.09 (1H, s).

MS (ES+) m/z 487 and 489 (MH⁺, 50 and 17%).

The free base of the title compound was dissolved in methanol and treated with excess 1M hydrogen chloride in diethyl ether then evaporated to dryness to give the title compound

### Example 26

### 7-Chloro-6-({4-[(6,7-dihydro[1,4]dioxino[2,3-c]pyridazin-3-ylmethyl)amino]-1-piperidinyl}methyl)-5,6-dihydro-4H-[1,3]thiazolo[5,4,3-ij]quinolin-2-one dihydrochloride

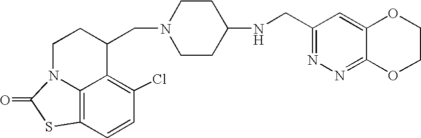

The title compound was prepared from 6-[(4-amino-1-piperidinyl)methyl]-7-chloro-5,6-dihydro-4H-[1,3]thiazolo[5,4,3-ij]quinolin-2-one (0.085 g, 0.25 mmole) and 6,7-dihydro[1,4]dioxino[2,3-c]pyridazine-3-carbaldehyde (for a preparation see Example 7g)) (0.041 g, 0.25 mmole) by the general method of Example 17c) to give the free base (0.075 g).

¹H NMR δ(CDCl₃) 1.51-1.67 (2H, m), 1.71-1.81 (1H, m), 1.92-2.11 (3H, m), 2.25-2.39 (2H, m), 2.45-2.53 (1H, s), 2.55-2.63 (1H, m), 2.69-2.79 (2H, m), 3.15-3.23 (1H, m), 3.39-3.54 (2H, m), 4.11 (2H, s), 4.21-4.29 (1H, s), 4.33-4.38 (2H, m), 4.48-4.53 (2H, s), 7.09 (1H, s), 7.11 (1H, d, J 2.8 Hz), 7.21 (1H, d, J 2.8 Hz).

MS (ES+) m/z 488 and 490 (MH⁺, 50 and 17%).

The free base of the title compound was dissolved in methanol and treated with excess 1M hydrogen chloride in diethyl ether. The resulting precipitate was separated from the supernatant liquid by decantation then dried to give the title compound.

### Example 27

### 6-({4-[(2,3-Dihydro [1,4]dioxino[2,3-c]pyridin-7-ylmethyl)amino]-1-piperidinyl}methyl)-5,6-dihydro-4H-[1,3]thiazolo[5,4,3-ij]quinolin-2-one dihydrochloride

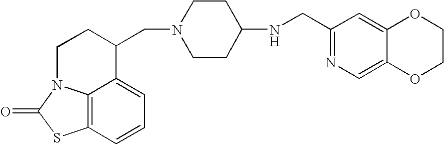

### a) 1,1-Dimethylethyl {1-[(2-oxo-5,6-dihydro-4H-[1,3]thiazolo[5,4,3-ij]quinolin-6-yl)methyl]-4-piperidinyl}carbamate

1,1-Dimethylethyl {1-[(7-chloro-2-oxo-5,6-dihydro-4H-[1,3]thiazolo[5,4,3-ij]quinolin-6-yl)methyl]-4-piperidinyl}carbamate (obtained by the general method of Example 25f) before deprotection with TFA) (0.1 g, 0.23 mmole) was hydrogenated in ethanol (30 mL) at S.T.P over 10% palladium on carbon paste (0.2 g) for 24 h. More catalyst (0.2 g) was added and the mixture hydrogenated for a further 24 h. The catalyst was removed by filtration and the filtrate evaporated to dryness to give the title compound (0.075 g).

MS (ES+) m/z 404 (MH⁺, 100%).

### b) 6-[(4-Amino-1-piperidinyl)methyl]-5,6-dihydro-4H-[1,3]thiazolo[5,4,3-ij]quinolin-2-one

1,1-Dimethylethyl {1-[(2-oxo-5,6-dihydro-4H-[1,3]thiazolo[5,4,3-ij]quinolin-6-yl)methyl]-4-piperidinyl}carbamate (0.075 g, 0.17 mmole) was dissolved in dichloromethane (10 mL) and treated with TFA (5 mL) and stirred at RT for 4 h. Reaction was proceeding slowly so was evaporated to dryness and the residue dissolved in 1,4-dioxane (10 mL) and treated with 5N hydrochloric acid (2 mL) and stirred at RT for 1 h. The mixture was then evaporated to dryness and the residue partitioned between saturated potassium carbonate solution and 10% methanol/dichloromethane. The organic phase was separated and dried over sodium sulphate, filtered and evaporated to dryness to give the title compound as an oil (0.04 g).

MS (ES+) m/z 304 (MH⁺, 100%).

### c) Title Compound

The title compound was prepared from 6-[(4-amino-1-piperidinyl)methyl]-5,6-dihydro-4H-[1,3]thiazolo[5,4,3-ij]quinolin-2-one (0.04 g, 0.12 mmole) and 2,3-dihydro[1,4]dioxino[2,3-c]pyridine-7-carboxaldehyde (0.02 g, 0.12 mmole) (for a synthesis see WO2004058144, Example 2(c) or WO2003087098 Example 19(d)) by the general method of Example 17c) to give the free base (0.035 g).

¹H NMR δ(CDCl₃) 1.45-1.68 (2H, m), 1.89-2.11 (3H, m), 2.12-2.25 (2H, m), 2.38-2.52 (2H, m), 2.58-2.69 (1H, m), 2.77-2.88 (1H, m), 2.93-3.01 (1H, m), 3.02-3.13 (1H, m), 3.68-3.91 (4H, m), 3.95-4.07 (1H, m), 4.21-4.37 (4H, m), 6.82 (1H, s), 7.05 (1H, t, J 4 Hz), 7.18 (1H, d, J 11.7 Hz), 7.21-7.28 (1H, m), 8.11 (1H, s).

MS (ES+) m/z 453 (MH⁺, 100%).

The free base of the title compound was dissolved in methanol and treated with excess 1M hydrogen chloride in diethyl ether then evaporated to dryness to give the title compound

### Example 28

### 6-({4-[(2,3-Dihydro [1,4]dioxino[2,3-c]pyridin-7-ylmethyl)amino]-1-piperidinyl}methyl)-7-fluoro-5,6-dihydro-4H-[1,3]thiazolo[5,4,3-ij]quinolin-2-one dihydrochloride

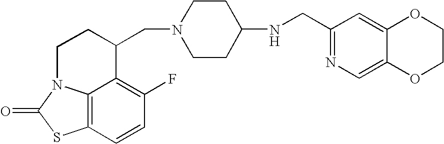

### a) 5-Fluoro-1,3-benzothiazol-2(3H)-one

A mixture of 1-chloro-4-fluoro-2-nitrobenzene (1.75 g, 10 mmole), sulphur flakes (1.6 g), THF (40 mL), water (1.1 mL) and TEA (5.6 mL) were placed in a stainless steel pressure bomb which was then pressurised with carbon monoxide to a pressure of 1000 kPA (15 bar) and heated at 90° C. for 12 h. After cooling and venting of the vessel the contents were evaporated to dryness. Chromatography on silica gel eluting with a methanol/DCM gradient gave the title compound (1 g, 59%).

MS (ES+) m/z 170 (MH⁺, 100%).

### b) Ethyl 3-(5-fluoro-2-oxo-1,3-benzothiazol-3(2H)-yl)propanoate

The title compound was prepared by the general method of Example 25a) from 5-fluoro-1,3-benzothiazol-2(3H)-one (0.44 g) to give the title compound (0.6 g, 86%).

MS (ES+) m/z 292 (M+Na, 100%).

### c) 3-(5-Fluoro-2-oxo-1,3-benzothiazol-3(2H)-yl)propanoic acid

Ethyl 3-(5-fluoro-2-oxo-1,3-benzothiazol-3(2H)-yl)propanoate (0.88 g, 3.3 mmole) was heated under reflux in 5N hydrochloric acid (40 mL) for 18 h. The mixture was allowed to cool and the product crystallised out. Filtration and drying gave the title compound as a crystalline solid (0.7 g, 89%).

MS (ES+) m/z 264 (M+Na, 30%).

### d) 7-Fluoro-4H-[1,3]thiazolo[5,4,3-ij]quinoline-2,6(5H)-dione

The title compound was prepared by the general method of Example 25c) from 3-(5-fluoro-2-oxo-1,3-benzothiazol-3(2H)-yl)propanoic acid (0.211 g, 0.87 mmole) to give product (0.184 g, 87%).

MS (ES+) m/z 224 (MH⁺, 100%).

### e) 7-Fluoro-6-[(methyloxy)methylidene]-5,6-dihydro-4H-[1,3]thiazolo[5,4,3-ij]quinolin-2-one

The title compound was prepared by the general method of Example 25d) from 7-fluoro-4H-[1,3]thiazolo[5,4,3-ij]quinoline-2,6(5H)-dione (0.184 g, 0.76 mmole) to give product (0.058 g, 30%).

MS (ES+) m/z 252 (MH⁺, 100%).

### f) 7-Fluoro-2-oxo-5,6-dihydro-4H-[1,3]thiazolo[5,4,3-ij]quinoline-6-carbaldehyde

The title compound was prepared by the general method of Example 25e) from 7-fluoro-6-[(methyloxy)methylidene]-5,6-dihydro-4H-[1,3]thiazolo[5,4,3-ij]quinolin-2-one (0.4 g, 1.6 mmole) to give product (0.36 g, 95%).

MS (ES+) m/z 270 (MH⁺ of methanol hemiacetal, 100%).

### g) 1,1-Dimethylethyl (2,3-dihydro[1,4]dioxino[2,3-c]pyridin-7-ylmethyl){1-[(7-fluoro-2-oxo-5,6-dihydro-4H-[1,3]thiazolo[5,4,3-ij]quinolin-6-yl)methyl]-4-piperidinyl}carbamate

The title compound was prepared by the general method of Example 17a) from 7-fluoro-2-oxo-5,6-dihydro-4H-[1,3]thiazolo[5,4,3-ij]quinoline-6-carbaldehyde (0.295 g, 1.2 mmole) and 1,1-dimethylethyl (2,3-dihydro[1,4]dioxino[2,3-c]pyridin-7-ylmethyl)4-piperidinylcarbamate (0.43 g, 1.2 mmole) (for a synthesis see WO2004058144, Example 99(h)) to give, after chromatography on silica gel eluting with an ethyl acetate/40-60 petroleum ether gradient, product (0.37 g, 52%).

MS (ES+) m/z 571 (MH⁺, 100%).

### h) Title Compound

1,1-Dimethylethyl (2,3-dihydro[1,4]dioxino[2,3-c]pyridin-7-ylmethyl){1-[(7-fluoro-2-oxo-5,6-dihydro-4H-[1,3]thiazolo[5,4,3-ij]quinolin-6-yl)methyl]-4-piperidinyl}carbamate (0.37 g, 0.65 mmole) in 1,4-dioxane (5 mL) was treated with 5N hydrochloric acid (1 mL) and stirred at RT for 18 h then at 40° C. for 4 h. The mixture was evaporated to dryness to give the title compound (0.346 g, 100%).

¹H NMR δ(DMSO-d₆) 1.79-1.91 (1H, m), 2.13-2.39 (3H, m), 2.79-2.86 (1H, m), 2.91-2.98 (1H, m), 2.99-3.17 (2H, m), 3.24-3.39 (1H, m), 3.41-3.58 (2H, m), 3.71-3.82 (2H, m), 3.83-4.39 (contains 5H+H₂O, m), 4.41-4.45 (2H, m), 4.46-4.51 (2H, m), 7.08 (1H, t, J 8.8 Hz), 7.49 (1H, s), 7.61-7.69 (1H, m), 8.38 (1H, s), 9.88-10.16 (2H, brs), 10.73-10.89 (1H, brs).

MS (ES+) m/z 471 (MH⁺, 80%).

### Example 29

### 6-({4-[(2,3-Dihydro [1,4]dioxino[2,3-c]pyridin-7-ylmethyl)amino]-1-piperidinyl}methyl)-7-fluoro-5,6-dihydro-4H-[1,3]thiazolo[5,4,3-ij]quinolin-2-one hydrochloride (Enantiomer 1) and

### Example 30

### 6-({4-[(2,3-Dihydro [1,4]dioxino[2,3-c]pyridin-7-ylmethyl)amino]-1-piperidinyl}methyl)-7-fluoro-5,6-dihydro-4H-[1,3]thiazolo[5,4,3-ij]quinolin-2-one hydrochloride (Enantiomer 2)

Racemic 6-({4-[(2,3-Dihydro[1,4]dioxino[2,3-c]pyridin-7-ylmethyl)amino]-1-piperidinyl}methyl)-7-fluoro-5,6-dihydro-4H-[1,3]thiazolo[5,4,3-ij]quinolin-2-one dihydrochloride (0.326 g) was chromatographed on a Chiralpak AD-H 21×250 mm column (multiple injections) eluting with a mixture of 15:65:20:0.1 methanol:acetonitrile:isopropyl alcohol:isopropylamine at 1 ml/min. The peak with retention time of 5.5 min was collected evaporated to give Enantiomer 1 (0.122 g) and the peak with retention time 8.8 min collected and evaporated to give Enantiomer 2 (0.132 g).

The free bases were slurried separately in methanol (5 mL) and treated with 1.0 equiv. of aqueous 6M HCl. The solvent was removed under reduced pressure and the solids dried under reduced pressure at 40° C. to give the corresponding hydrochloride salts, each with >99% ee.

### Example 31

### 6-({4-[(6,7-Dihydro [1,4]dioxino[2,3-c]pyridazin-3-ylmethyl)amino]-1-piperidinyl}methyl)-7-fluoro-5,6-dihydro-4H-[1,3]thiazolo[5,4,3-ij]quinolin-2-one dihydrochloride

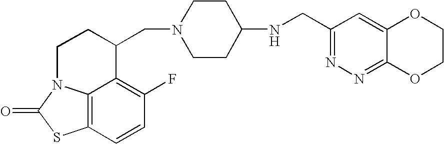

### a) 1,1-Dimethylethyl {1-[(7-fluoro-2-oxo-5,6-dihydro-4H-[1,3]thiazolo[5,4,3-ij]quinolin-6-yl)methyl]-4-piperidinyl}carbamate

The title compound was prepared from 7-fluoro-2-oxo-5,6-dihydro-4H-[1,3]thiazolo[5,4,3-ij]quinoline-6-carbaldehyde (for a preparation see Example 28f)) (0.06 g, 0.25 mmole) and 4-(N-Boc-amino)piperidine (0.05 g, 0.25 mmole) by the general method of Example 17a) to give product (0.068 g, 64%).

MS (ES+) m/z 422 (MH⁺, 100%).

### b) 6-[(4-Amino-1-piperidinyl)methyl]-7-fluoro-5,6-dihydro-4H-[1,3]thiazolo[5,4,3-ij]quinolin-2-one

The title compound was prepared from 1,1-dimethylethyl {1-[(7-fluoro-2-oxo-5,6-dihydro-4H-[1,3]thiazolo[5,4,3-ij]quinolin-6-yl)methyl]-4-piperidinyl}carbamate (0.068 g, 0.16 mmole) by the general method of Example 27b) to give product (0.05 g).

MS (ES+) m/z 322 (MH⁺, 100%).

### c) Title Compound

The title compound was prepared from 6-[(4-amino-1-piperidinyl)methyl]-7-fluoro-5,6-dihydro-4H-[1,3]thiazolo[5,4,3-ij]quinolin-2-one (0.05 g, 0.15 mmole) and 6,7-dihydro[1,4]dioxino[2,3-c]pyridazine-3-carbaldehyde (for a preparation see Example 7g)) (0.026 g, 0.15 mmole) by the general method of Example 17c) to give the free base (0.046 g). This was dissolved in methanol and treated with excess 1M hydrogen chloride in diethyl ether then evaporated to dryness to give the title compound.

¹H NMR δ(DMSO-d₆) 1.75-1.97 (1H, m), 2.15-2.46 (3H, m), 2.72-3.19 (4H, m), 3.29-3.65 (3H, m), 3.69-3.85 (2H, m), 3.91-4.65 (contains 9H+H₂O, m), 7.08 (1H, app. t, J 10 Hz), 7.53-7.65 (2H, m), 10.14 (2H, br.s), 10.83 (1H, br.s).

MS (ES+) m/z 472 (MH⁺, 45%).

### Example 32

### 7-Fluoro-6-[(4-{[(3-oxo-3,4-dihydro-2H-pyrido[3,2-b][1,4]oxazin-6-yl)methyl]amino}-1-piperidinyl)methyl]-5,6-dihydro-4H-[1,3]thiazolo[5,4,3-ij]quinolin-2-one dihydrochloride

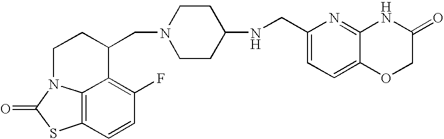

The title compound was prepared from 6-[(4-amino-1-piperidinyl)methyl]-7-fluoro-5,6-dihydro-4H-[1,3]thiazolo[5,4,3-ij]quinolin-2-one (for a preparation see Example 31b)) (0.043 g, 0.13 mmole) and 3-oxo-3,4-dihydro-2H-pyrido[3,2-b][1,4]oxazine-6-carboxaldehyde (0.023 g, 0.13 mmole) (for a synthesis see WO2003087098 Example 31(e)) by the general method of Example 17c) to give the free base (0.057 g, 89%).

¹H NMR δ(CDCl₃) 1.38-1.57 (2H, m), 1.65-2.06 (4H, m), 2.13-2.27 (1H, m), 2.35-2.43 (2H, m), 2.45-2.58 (2H, m), 2.67-2.79 (1H, m), 2.98-3.11 (1H, m), 3.34-3.58 (2H, m), 3.81 (2H, s), 4.22-4.32 (1H, s), 4.61 (2H, s), 6.83 (1H, app. t, J 8.8 Hz), 6.93 (1H, d, J 8 Hz), 7.15-7.26 (2H, m).

MS (ES+) m/z 484 (MH⁺, 100%).

The free base of the title compound was dissolved in methanol and treated with excess 4M hydrogen chloride in 1,4-dioxane then evaporated to dryness to give the title compound

### Example 33

### 7-Fluoro-6-[(4-{[(3-oxo-3,4-dihydro-2H-pyrido[3,2-b][1,4]thiazin-6-yl)methyl]amino}-1-piperidinyl)methyl]-5,6-dihydro-4H-[1,3]thiazolo[5,4,3-ij]quinolin-2-one dihydrochloride

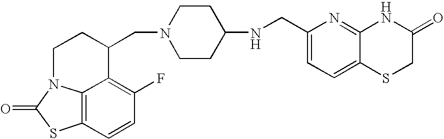

The title compound was prepared from 6-[(4-amino-1-piperidinyl)methyl]-7-fluoro-5,6-dihydro-4H-[1,3]thiazolo[5,4,3-ij]quinolin-2-one (for a preparation see Example 31b)) (0.043 g, 0.13 mmole) and 3-oxo-3,4-dihydro-2H-pyrido[3,2-b][1,4]thiazine-6-carboxaldehyde (0.026 g, 0.13 mmole) (for a synthesis see WO2004058144 Example 7(d)) by the general method of Example 17c) to give the free base (0.064 g, 95%).

¹H NMR δ(CDCl₃) 1.37-1.57 (2H, m), 1.71-2.25 (5H, m), 2.35-2.43 (2H, m), 2.45-2.58 (2 h, m), 2.68-2.79 (1H, m), 2.97-3.11 (1H, m), 3.35-3.57 (4H, m, including 3.41 (2H, s)), 3.82 (2H, s), 4.19-4.31 (1H, m), 6.83 (1H, app. t, J 9 Hz), 7.99 (1H, d, J 7.8 Hz), 7.15-7.23 (1H, m), 7.57 (1H, d, J 7.8 Hz).

MS (ES+) m/z 500 (MH⁺, 100%).

The free base of the title compound was dissolved in methanol and treated with excess 4M hydrogen chloride in 1,4-dioxane then evaporated to dryness to give the title compound.

### Example 34

### 6-({(3R,4S)-4-[(2,3-dihydro[1,4]dioxino[2,3-c]pyridin-7-ylmethyl)amino]-3-hydroxy-1-piperidinyl}methyl)-7-fluoro-5,6-dihydro-4H-[1,3]thiazolo[5,4,3-ij]quinolin-2-one dihydrochloride

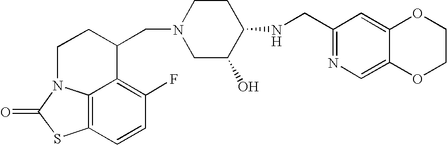

### a) 1,1-Dimethylethyl {(3R,4S)-1-[(7-fluoro-2-oxo-5,6-dihydro-4H-[1,3]thiazolo[5,4,3-ij]quinolin-6-yl)methyl]-3-hydroxy-4-piperidinyl}carbamate

The title compound was prepared from 7-fluoro-2-oxo-5,6-dihydro-4H-[1,3]thiazolo[5,4,3-ij]quinoline-6-carbaldehyde (for a preparation see Example 28f)) (0.093 g, 0.39 mmole) and 1,1-dimethylethyl [(3R,4S)-3-hydroxy-4-piperidinyl]carbamate (0.085 g, 0.39 mmole) (cis-(3-hydroxy-piperidin-4-yl)-carbamic acid tert-butyl ester Enantiomer 1 (WO 2004058144 Example 5(c)) by the general method of Example 17a) to give product (0.12 g, 70%).

MS (ES+) m/z 438 (MH⁺, 100%).

### b) 6-{[(3R,4S)-4-Amino-3-hydroxy-1-piperidinyl]methyl}-7-fluoro-5,6-dihydro-4H-[1,3]thiazolo[5,4,3-ij]quinolin-2-one

The title compound was prepared from 1,1-dimethylethyl {(3R,4S)-1-[(7-fluoro-2-oxo-5,6-dihydro-4H-[1,3]thiazolo[5,4,3-ij]quinolin-6-yl)methyl]-3-hydroxy-4-piperidinyl}carbamate (0.12 g, 0.27 mmole) by the general method of Example 27b) to give product (0.12 g).

MS (ES+) m/z 338 (MH⁺, 100%).

### c) Title Compound

The title compound was prepared from 6-{[(3R,4S)-4-amino-3-hydroxy-1-piperidinyl]methyl}-7-fluoro-5,6-dihydro-4H-[1,3]thiazolo[5,4,3-ij]quinolin-2-one (0.27 mmole) and 2,3-dihydro[1,4]dioxino[2,3-c]pyridine-7-carboxaldehyde (0.04 g, 0.24 mmole) (for a synthesis see WO2004058144 Example 2(c) or WO2003087098 Example 19(d)) by the general method of Example 17c) to give the free base (0.064 g, 49%).

¹H NMR δ(CDCl₃) 1.72-1.89 (2H, m), 1.95-2.11 (1H, m), 2.15-2.28 (1H, m), 2.35-2.53 (2H, m), 2.75-3.06 (3H, m), 3.09-3.19 (1H, m), 3.31-3.68 (7H, m), 3.95 (2H, s), 4.21-4.39 (4H, m), 6.84 (1H, t, J 8.8 Hz), 6.85 (1H, s), 7.15-7.25 (1H, m), 8.11 (1H, s).

MS (ES+) m/z 487 (MH⁺, 100%).

The free base of the title compound was dissolved in methanol and treated with excess 4M hydrogen chloride in 1,4-dioxane then evaporated to dryness to give the title compound.

### Example 35

### 7-({4-[(2,3-Dihydro[1,4]dioxino[2,3-c]pyridin-7-ylmethyl)amino]-1-piperidinyl}methyl)-8-fluoro-6,7-dihydro-5H-[1,4]oxazino [2,3,4-ij]quinolin-3(2H)-one dihydrochloride

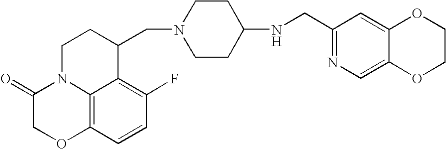

### a) 3-(6-Fluoro-3-oxo-2,3-dihydro-4H-1,4-benzoxazin-4-yl)propanoic acid

A solution of 6-fluoro-2H-1,4-benzoxazin-3(4H)-one (0.5 g, 3 mmoles) in DMF (5 mL) was treated with sodium hydride 60% dispersion in oil (0.14 g, 3.6 mmoles) and stirred at RT for 30 mins. Propiolactone (0.24 g, 3.3 mmoles) was then added and the mixture stirred at RT for a further 3 h. 5M Hydrochloric acid (5 mL) was then added and the mixture evaporated to dryness. The residue was partitioned between ethyl acetate (20 mL) and water (2×20 mL), the organic layer was then extracted with 2N NaOH (2×7 mL) and the combined basic extracts acidified with concentrated hydrochloric acid. The resulting solid was filtered off and washed with water (2×5 mL) then dried in-vacuo at 50° C. to give a white solid (0.582 g).

MS (ES+) m/z 240 (MH⁺ 25%).

### b) 8-Fluoro-5H-[1,4]oxazino [2,3,4-ij]quinoline-3,7(2H,6H)-dione

A suspension of 3-(6-fluoro-3-oxo-2,3-dihydro-4H-1,4-benzoxazin-4-yl)propanoic acid (0.2 g, 0.83 mmoles) in DCM (20 mL) was treated with oxalyl chloride (0.15 mL, 1.7 mmoles) and DMF (1 drop) and stirred at RT for 1 h. The mixture was evaporated to dryness and the residue re-dissolved in DCM (20 mL) and treated with aluminium chloride (0.23 g, 1.7 mmoles) and stirred at RT for 30 mins. Water (30 mL) was added and the mixture extracted with 10% methanol/DCM (2×30 mL). The organic extracts were dried over Na₂SO₄, filtered and evaporated to dryness. The residue was chromatographed on silica gel eluting with a gradient of 0-10% methanol/DCM to give the title compound as an off-white solid (0.18 g).

MS (ES+) m/z 222 (MH⁺ 100%).

### c) 8-Fluoro-7-[(methyloxy)methylidene]-6,7-dihydro-5H-[1,4]oxazino[2,3,4-ij]quinolin-3(2H)-one

A suspension of triphenyl(methoxymethyl) phosphonium chloride (0.84 g, 2.4 mmoles) in 1,4-dioxane (20 mL) was treated with potassium tert-butoxide (0.275 g, 2.4 mmoles) and stirred at RT under an argon atmosphere for 30 mins. 8-Fluoro-5H-[1,4]oxazino[2,3,4-ij]quinoline-3,7(2H,6H)-dione (0.18 g, 0.8 mmoles) was dissolved in toluene (20 mL) then evaporated to dryness then dissolved in 1,4-dioxane (20 mL) and added to the mixture and stirred at RT for a further 18 h. Water (50 mL) was then added and the mixture extracted with ethyl acetate (2×50 mL). The combined extracts were washed with saturated sodium chloride solution (50 mL) then dried over Na₂SO₄, filtered and evaporated to dryness. Chromatography on silica gel eluting with a gradient of 0-50% ethyl acetate/40-60 petroleum ether gave the title compound as a pale orange crystalline solid (0.134 g).

MS (ES+) m/z 250 (MH⁺ 100%).

### d) 8-Fluoro-3-oxo-2,3,6,7-tetrahydro-5H-[1,4]oxazino[2,3,4-ij]quinoline-7-carbaldehyde

To a solution of 8-fluoro-7-[(methyloxy)methylidene]-6,7-dihydro-5H-[1,4]oxazino[2,3,4-ij]quinolin-3(2H)-one (0.134 g, 0.54 mmoles) in acetonitrile (7 mL) was added sodium iodide (0.24 g, 1.6 mmoles) and trimethylsilyl chloride (0.3 mL) and the mixture stirred at RT for 18 h. Water (20 mL) was added along with sodium metabisulphite (2 g) then the mixture was extracted with ethyl acetate (2×30 mL). The combined organics were dried over Na₂SO₄, filtered and evaporated to dryness to give the title compound as a pale orange oil (0.105 g).

MS (ES+) m/z 236 (MH⁺ 100%).

### e) 1,1-Dimethylethyl (2,3-dihydro[1,4]dioxino[2,3-c]pyridin-7-ylmethyl){1-[(8-fluoro-3-oxo-2,3,6,7-tetrahydro-5H-[1,4]oxazino[2,3,4-ij]quinolin-7-yl)methyl]-4-piperidinyl}carbamate

To a solution of 8-fluoro-3-oxo-2,3,6,7-tetrahydro-5H-[1,4]oxazino[2,3,4-ij]quinoline-7-carbaldehyde (0.2 g, 0.85 mmoles) in chloroform (7 mL) and methanol (7 mL) was added 1,1-dimethylethyl (2,3-dihydro[1,4]dioxino[2,3-c]pyridin-7-ylmethyl)4-piperidinylcarbamate (for a synthesis see WO2004058144 Example 99(h)) (0.296 g, 0.85 mmoles) and acetic acid (3 drops). (Polystyrylmethyl)trimethylammonium cyanoborohydride 4.0 mmoles/gm (1.75 g, 7 mmoles) was added and the mixture stirred at RT for 18 h. The mixture was filtered to remove resin washing with 1:1 methanol/DCM (3×10 mL). The combined filtrate and washings were evaporated to dryness then chromatographed on silica gel eluting with a gradient of 0-12% methanol/DCM to give a mixture containing the title compound as a light brown oil (0.18 g).

MS (ES+) m/z 569 (MH⁺ 30%).

### f) Title Compound

A solution of 1,1-dimethylethyl (2,3-dihydro[1,4]dioxino[2,3-c]pyridin-7-ylmethyl){1-[(8-fluoro-3-oxo-2,3,6,7-tetrahydro-5H-[1,4]oxazino[2,3,4-ij]quinolin-7-yl)methyl]-4-piperidinyl}carbamate (0.18 g, 0.3 mmoles) in DCM (20 mL) was treated with trifluoroacetic acid (20 mL) and left to stand at RT for 18 h. The mixture was then evaporated to dryness and the residue partitioned between 20% potassium carbonate in water (10 mL) and 10% methanol/DCM (2×15 mL). The combined organics were then dried over Na₂SO₄ filtered and evaporated to dryness. Chromatography on silica gel eluting with a gradient of 0-8% 2M ammonia/methanol/DCM gave the free base of the title compound as a light brown oil (0.075 g).

NMR 250 MHz (CDCl₃) δ: 1.35-1.75 (3H, m), 1.81-2.04 (4H, m), 2.10-2.28 (1H, m), 2.31-2.59 (4H, m), 2.65-2.75 (1H, m), 2.97-3.08 (1H, m), 3.18-3.35 (2H, m), 3.79 (2H, s), 4.22-4.42 (5H, m), 4.57 (2H, s), 6.62 (1H, t, J 8.7 Hz), 6.73-6.81 (1H, m), 6.83 (1H, s), 8.11 (1H, s).

MS (ES+) m/z 469 (MH⁺, 40%).

The free base of the title compound was dissolved in 1:1 DCM/methanol (10 mL) and treated with an excess of 4.0M hydrogen chloride in 1,4-dioxane then evaporated to dryness to give the title compound as a light brown solid (0.086 g).

### Example 36

### 7-({4-[(6,7-Dihydro [1,4]dioxino[2,3-c]pyridazin-3-ylmethyl)amino]-1-piperidinyl}methyl)-8-fluoro-6,7-dihydro-5H-[1,4]oxazino [2,3,4-ij]quinolin-3(2H)-one dihydrochloride

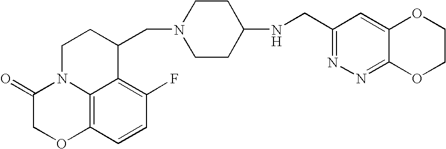

### a) 1,1-Dimethylethyl (6,7-dihydro[1,4]dioxino[2,3-c]pyridazin-3-ylmethyl){1-[(8-fluoro-3-oxo-2,3,6,7-tetrahydro-5H-[1,4]oxazino[2,3,4-ij]quinolin-7-yl)methyl]-4-piperidinyl}carbamate

To a solution of 8-fluoro-3-oxo-2,3,6,7-tetrahydro-5H-[1,4]oxazino[2,3,4-ij]quinoline-7-carbaldehyde (for a preparation see Example 35(d)) (0.2 g, 0.85 mmoles) in chloroform (7 mL) and methanol (7 mL) was added 1,1-dimethylethyl (6,7-dihydro[1,4]dioxino[2,3-c]pyridazin-3-ylmethyl)4-piperidinylcarbamate (for a preparation see WO2008009700 Example 44A (c)) (0.298 g, 0.85 mmoles) and acetic acid (3 drops). (Polystyrylmethyl)trimethylammonium cyanoborohydride 4.0 mmoles/gm (1.75 g, 7 mmoles) was added and the mixture stirred at RT for 18 h. The mixture was filtered to remove resin washing with 1:1 methanol/DCM (3×10 mL). The combined filtrate and washings were evaporated to dryness then chromatographed on silica gel eluting with a gradient of 0-12% methanol/DCM to give a mixture containing the title compound as a light brown oil (0.15 g).

MS (ES+) m/z 570 (MH⁺ 80%).

### b) Title Compound

A solution of 1,1-dimethylethyl (6,7-dihydro[1,4]dioxino[2,3-c]pyridazin-3-ylmethyl){1-[(8-fluoro-3-oxo-2,3,6,7-tetrahydro-5H-[1,4]oxazino[2,3,4-ij]quinolin-7-yl)methyl]-4-piperidinyl}carbamate in DCM (20 mL) was treated with trifluoroacetic acid (20 mL) and left to stand at RT for 18 h. The mixture was then evaporated to dryness and the residue partitioned between 20% potassium carbonate in water (10 mL) and 10% methanol/DCM (2×15 mL). The combined organics were then dried over Na₂SO₄ filtered and evaporated to dryness. Chromatography on silica gel eluting with a gradient of 0-8% 2M ammonia/methanol/DCM gave the free base of the title compound as a light brown oil (0.06 g).

NMR 250 MHz (CDCl₃) δ: 1.33-1.58 (2H, m), 1.65-2.11 (5H, m), 2.10-2.28 (1H, m), 2.35-2.62 (4H, m), 2.65-2.75 (1H, m), 2.97-3.08 (1H, m), 3.18-3.35 (2H, m), 4.01 (2H, s), 4.33-4.68 (7H, m), 6.62 (1H, t, J 8.7 Hz), 6.71-6.80 (1H, m), 7.04 (1H, s).

MS (ES+) m/z 470 (MH⁺, 80%).

The free base of the title compound was dissolved in 1:1 DCM/methanol (10 mL) and treated with an excess of 4.0M hydrogen chloride in 1,4-dioxane then evaporated to dryness to give the title compound as a light brown solid (0.069 g).

### Example 37

### 8-Fluoro-7-[(4-{[(3-oxo-3,4-dihydro-2H-pyrido[3,2-b][1,4]oxazin-6-yl)methyl]amino}-1-piperidinyl)methyl]-6,7-dihydro-5H-[1,4]oxazino [2,3,4-ij]quinolin-3(2H)-one dihydrochloride

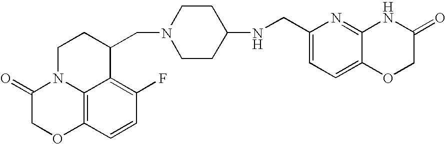

### a) 1,1-Dimethylethyl {1-[(8-fluoro-3-oxo-2,3,6,7-tetrahydro-5H-[1,4]oxazino[2,3,4-ij]quinolin-7-yl)methyl]-4-piperidinyl}carbamate

To a solution of 8-fluoro-3-oxo-2,3,6,7-tetrahydro-5H-[1,4]oxazino[2,3,4-ij]quinoline-7-carbaldehyde (for a preparation see Example 35(d)) (0.2 g, 0.85 mmoles) in chloroform (7 mL) and methanol (7 mL) was added 4-N-Boc aminopiperidine (0.17 g, 0.85 mmoles) and acetic acid (3 drops). (Polystyrylmethyl)trimethylammonium cyanoborohydride 4.0 mmoles/gm (1.75 g, 7 mmoles) was added and the mixture stirred at RT for 18 h. The mixture was filtered to remove resin washing with 1:1 methanol/DCM (3×10 mL). The combined filtrate and washings were evaporated to dryness then chromatographed on silica gel eluting with a gradient of 0-12% methanol/DCM to give a mixture containing the title compound as a light brown oil (0.25 g).

MS (ES+) m/z 420 (MH⁺, 100%).

### b) 7-[(4-Amino-1-piperidinyl)methyl]-8-fluoro-6,7-dihydro-5H-[1,4]oxazino[2,3,4-ij]quinolin-3(2H)-one

A solution of 1,1-dimethylethyl {1-[(8-fluoro-3-oxo-2,3,6,7-tetrahydro-5H-[1,4]oxazino[2,3,4-ij]quinolin-7-yl)methyl]-4-piperidinyl}carbamate (0.25 g, 0.6 mmoles) in DCM (20 mL) was treated with trifluoroacetic acid (20 mL) and left to stand at RT for 18 h. The mixture was then evaporated to dryness and the residue partitioned between 20% potassium carbonate in water (10 mL) and 10% methanol/DCM (2×15 mL). The combined organics were then dried over Na₂SO₄ filtered and evaporated to dryness. Chromatography on silica gel eluting with a gradient of 0-8% 2M ammonia/methanol/DCM gave the title compound as a light brown oil (0.116 g).

MS (ES+) m/z 320 (MH⁺, 100%).

### c) Title Compound

To a solution of 7-[(4-amino-1-piperidinyl)methyl]-8-fluoro-6,7-dihydro-5H-[1,4]oxazino[2,3,4-ij]quinolin-3(2H)-one (0.058 g, 0.18 mmoles) in methanol (4 mL) was added 3-oxo-3,4-dihydro-2H-pyrido[3,2-b][1,4]oxazine-6-carbaldehyde (for a synthesis see WO2003087098 Example 31(e)) (0.032 g, 0.18 mmoles) and acetic acid (2 drops). (Polystyrylmethyl)trimethylammonium cyanoborohydride 4.0 mmoles/gm (0.35 g, 1.4 mmoles) was added and the mixture stirred at RT for 18 h. The mixture was filtered to remove resin washing with 1:1 methanol/DCM (3×10 mL). The combined filtrate and washings were evaporated to dryness then chromatographed on silica gel eluting with a gradient of 0-8% 2M ammonia/methanol/DCM to give the free base of the title compound as a colourless oil (0.081 g).

NMR 250 MHz (CDCl₃) δ: 1.37-1.74 (4H, m), 1.81-2.05 (3H, m), 2.15-2.28 (1H, m), 2.35-2.58 (3H, m), 2.68-2.78 (1H, m), 2.95-3.38 (3H, m), 3.85 (2H, s), 4.35-4.45 (1H, m), 4.58 (H, s), 4.65 (2H, s), 6.62 (1H, t, J 9 Hz), 6.74-6.79 (1H, m), 6.94 (1H, d, J 8 Hz), 7.2 (1H, d, J 8 Hz).

MS (ES+) m/z 482 (MH⁺, 100%).

The free base of the title compound was dissolved in 1:1 DCM/methanol (5 mL) and treated with an excess of 4.0M hydrogen chloride in 1,4-dioxane then evaporated to dryness to give the title compound as a white solid (0.084 g).

### Example 38

### 8-Fluoro-7-{[(3S,4S)-3-hydroxy-4-({[(3-oxo-3,4-dihydro-2H-pyrido[3,2-b][1,4]oxazin-6-yl)methyl]amino}methyl)-1-pyrrolidinyl]methyl}-6,7-dihydro-5H-[1,4]oxazino [2,3,4-ij]quinolin-3(2H)-one dihydrochloride

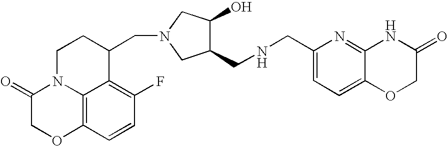

### a) Phenylmethyl ({(3S,4S)-1-[(8-fluoro-3-oxo-2,3,6,7-tetrahydro-5H-[1,4]oxazino[2,3,4-ij]quinolin-7-yl)methyl]-4-hydroxy-3-pyrrolidinyl}methyl)carbamate

To a solution/suspension of 8-fluoro-3-oxo-2,3,6,7-tetrahydro-5H-[1,4]oxazino[2,3,4-ij]quinoline-7-carbaldehyde (for a preparation see Example 35(d)) (0.76 g, 3.2 mmoles) in methanol (50 mL) was added phenylmethyl {[(3R,4S)-4-hydroxy-3-pyrrolidinyl]methyl}carbamate (for a synthesis see WO2008009700 Example 61(a)) (0.8 g, 3.2 mmoles) and acetic acid (catalytic). (Polystyrylmethyl)trimethylammonium cyanoborohydride 4.0 mmoles/gm (5 g, 19.2 mmoles) was added and the mixture stirred at RT for 18 h then heated at 40° C. for 72 h. The mixture was filtered to remove resin washing with 1:1 methanol/DCM (2×50 mL). The combined filtrate and washings were evaporated to dryness then chromatographed on silica gel eluting with a gradient of 0-8% 2M ammonia/methanol/DCM to give the title compound as a pale cream foam (0.45 g).

MS (ES+) m/z 470 (MH⁺ 100%).

### b) 7-{[(3S,4S)-3-(Aminomethyl)-4-hydroxy-1-pyrrolidinyl]methyl}-8-fluoro-6,7-dihydro-5H-[1,4]oxazino[2,3,4-ij]quinolin-3(2H)-one

Phenylmethyl ({(3S,4S)-1-[(8-fluoro-3-oxo-2,3,6,7-tetrahydro-5H-[1,4]oxazino[2,3,4-ij]quinolin-7-yl)methyl]-4-hydroxy-3-pyrrolidinyl}methyl)carbamate (0.45 g, 0.95 mmoles) was hydrogenated at S.T.P in ethanol (30 mL) over a 10% palladium on charcoal paste (0.3 g) for 4 h. The catalyst was removed by filtration and the filtrate evaporated to dryness to give the title compound as a white foam (0.3 g).

MS (ES+) m/z 336 (MH⁺ 100%).

### c) Title Compound

To a solution of 7-{[(3S,4S)-3-(Aminomethyl)-4-hydroxy-1-pyrrolidinyl]methyl}-8-fluoro-6,7-dihydro-5H-[1,4]oxazino[2,3,4-ij]quinolin-3(2H)-one (0.1 g, 0.3 mmoles) in methanol (5 mL) and acetic acid (2 drops) was added 3-oxo-3,4-dihydro-2H-pyrido[3,2-b][1,4]oxazine-6-carbaldehyde (for a synthesis see WO2003087098 Example 31(e)) (0.05 g, 0.28 mmoles). (Polystyrylmethyl)trimethylammonium cyanoborohydride 4.0 mmoles/gm (0.6 g, 2.4 mmoles) was added and the mixture stirred at RT for 18 h. The mixture was filtered to remove resin washing with 1:1 methanol/DCM (3×10 mL). The combined filtrate and washings were evaporated to dryness then chromatographed on silica gel eluting with a gradient of 0-10% 2M ammonia/methanol/DCM to give the free base of the title compound as a colourless oil (0.075 g).

NMR 250 MHz (CDCl₃) δ: 2.35-3.02 (12H, m), 3.35-3.69 (2H, m), 3.78-3.82 (2H, m), 4.35-4.48 (2H, m), 4.58 (2H, s), 4.64 (2H, s), 6.62 (1H, t, J 9 Hz), 6.74-6.79 (1H, m), 6.88 (1H, d, J 8 Hz), 7.21 (1H, d, J 8 Hz).

MS (ES+) m/z 498 (MH⁺, 100%).

The free base of the title compound was dissolved in 1:1 DCM/methanol (5 mL) and treated with an excess of 4.0M hydrogen chloride in 1,4-dioxane then evaporated to dryness to give the title compound as a white solid (0.08 g).

### Example 39

### 8-Fluoro-7-{[(3S,4S)-3-hydroxy-4-({[(3-oxo-3,4-dihydro-2H-pyrido[3,2-b][1,4]thiazin-6-yl)methyl]amino}methyl)-1-pyrrolidinyl]methyl}-6,7-dihydro-5H-[1,4]oxazino [2,3,4-ij]quinolin-3(2H)-one dihydrochloride

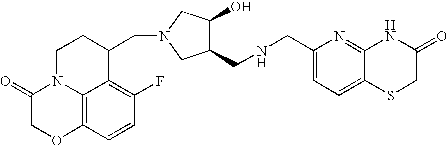

To a solution of 7-{[(3S,4S)-3-(aminomethyl)-4-hydroxy-1-pyrrolidinyl]methyl}-8-fluoro-6,7-dihydro-5H-[1,4]oxazino[2,3,4-ij]quinolin-3(2H)-one (for a preparation see Example 38(b)) (0.1 g, 0.3 mmoles) in methanol (5 mL) and acetic acid (2 drops) was added 3-oxo-3,4-dihydro-2H-pyrido[3,2-b][1,4]thiazine-6-carbaldehyde (for a synthesis see WO2004058144 Example 7(d)) (0.055 g, 0.28 mmoles). (Polystyrylmethyl)trimethylammonium cyanoborohydride 4.0 mmoles/gm (0.6 g, 2.4 mmoles) was added and the mixture stirred at RT for 18 h. The mixture was filtered to remove resin washing with 1:1 methanol/DCM (3×10 mL). The combined filtrate and washings were evaporated to dryness then chromatographed on silica gel eluting with a gradient of 0-10% 2M ammonia/methanol/DCM to give the free base of the title compound as a colourless oil (0.093 g).

NMR 250 MHz (CDCl₃) δ: 1.61-1.77 (2H, m), 2.32-3.05 (12H, m), 3.17-3.35 (2H, m), 3.85-3.90 (2H, m), 4.38-4.51 (2H, m), 4.59 (2H, s), 6.62 (1H, t, J 9 Hz), 6.74-6.79 (1H, m), 6.88 (1H, d, J 8 Hz), 7.61 (1H, d, J 8 Hz).

MS (ES+) m/z 514 (MH⁺, 100%).

The free base of the title compound was dissolved in 1:1 DCM/methanol (5 mL) and treated with an excess of 4.0M hydrogen chloride in 1,4-dioxane then evaporated to dryness to give the title compound as a white solid (0.107 g).

### Biological Activity

### Antimicrobial Activity Assay:

Whole-cell antimicrobial activity was determined by broth microdilution using the Clinical and Laboratory Standards Institute (CLSI) recommended procedure, Document M7-A7, “Methods for Dilution Susceptibility Tests for Bacteria that Grow Aerobically”.

The compounds were tested in serial two-fold dilutions ranging from 0.016 to 16 mcg/mL.

Compounds were evaluated against Gram-positive organisms selected from Staphylococcus aureus, Streptococcus pneumoniae, Streptococcus pyogenes, Enterococcus faecalis and Enterococcus faecium.

In addition, compounds were evaluated against Gram-negative organisms selected from Haemophilus influenzae, Moraxella catarrhalis, Escherichia coli, Pseudomonas aeruginosa, Proteus mirabilis, Legionella pneumophila, Chlamydia pneumoniae, Enterobacter cloacae, Enterobacter aerogenes, Klebsiella pneumoniae and Stenotrophomonas maltophilia.

The L. pneumophila isolates were tested using a modified CLSI procedure for broth microdilution. For this assay, compounds were tested in serial doubling dilutions over a concentration range of 0.03 to 32 mcg/mL. An inoculum of each test isolate was prepared in buffered yeast broth and adjusted to a density equivalent to a 0.5 McFarland standard. After inoculation, the microtitre plates were incubated at 37° C. for 72 hours.

For the C. pneumoniae isolates, stocks were thawed and diluted in CCM to yield an inoculum containing ˜1×10⁴ inclusion forming units/ml (IFUs/ml). A 100 μL aliquot of the inoculum was added to all wells of a microtitre plate containing HEp-2 cells grown to confluence. Microtitre plates were centrifuged for 1 hour at 1700 g., then incubated for 1 hour at 35° C. in 5% CO₂. One hundred microliters of diluted test compounds, prepared as a 2-fold dilution series in CCM/cycloheximide was then added to the microtiter plates. After 72 hours incubation at 35° C. in 5% CO₂, the microtitre plates were stained with a murine monoclonal fluorescein-conjugated antibody (Kallestad Cat. #532 Roche Biomedical Products) in accordance with the manufacturer recommendations. Upon staining, the IFUs produced an apple-green color, visible against the red counter stained HEp-2 cells when viewed at 100× magnification. The MIC was defined as the lowest concentration of compound at which no IFUs were seen.

The minimum inhibitory concentration (MIC) was determined as the lowest concentration of compound that inhibited visible growth. A mirror reader was used to assist in determining the MIC endpoint.

Each of the listed Examples, as identified in the present application, were tested in at least one exemplified salt or free base form. Unless otherwise noted, the listed Examples had a MIC ≦2 μg/ml against a strain of at least one of the organisms listed above. For at least one strain of every organism listed above, at least one Example had a MIC ≦2 μg/ml with the exception of strains of Proteus mirabilis for which at least one example had a MIC ≦8 μg/ml, and Pseudomonas and Chlamydia pneumoniae for which at least one Example had a MIC ≦16 μg/ml.

Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 (Difco catalogue Ref. 271310)+ADC medium (Becton Dickinson Catalogue Ref. 211887). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9+ADC medium and 0.025% Tween 80 (Sigma P4780), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

Results of the Mycobacterium tuberculosis H37Rv Inhibition Assay

Examples 8, 10, 11, 16, 17, 19-21, 23-27, 31, 32 and 34-39 were tested in the Mycobacterium tuberculosis H37Rv inhibition assay. Examples 10, 11, 17, 32 and 37 showed an MIC value of 1.8 μg/ml or lower. Examples 11, 17 and 37 showed an MIC value of 1.0 μg/ml or lower.

1. A compound of formula (I) or a pharmaceutically acceptable salt and/or N-oxide thereof: 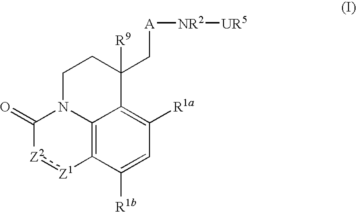
wherein:
Z¹ and Z² together are CH═CH or S or Z¹ is O and Z² is CH₂;
R^{1a} and R^{1b} are independently selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; or aminocarbonyl wherein the amino group is optionally substituted by (C₁₋₄)alkyl;
R² is hydrogen, or (C₁₋₄)alkyl, or together with R⁶ forms Y as defined below;
A is a group (i): 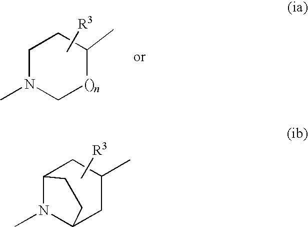
in which: R³ is as defined for R^{1a} or R^{1b} or is oxo and n is 1 or 2:
or A is a group (ii) 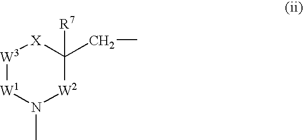
W¹, W² and W³ are CR⁴R⁸;
or W² and W³ are CR⁴R⁸ and W¹ represents a bond between W³ and N;
X is O, CR⁴R⁸, or NR⁶;
one R⁴ is as defined for R^{1a} and R^{1b} and the remainder and R⁸ are hydrogen or one R⁴ and R⁸ are together oxo and the remainder are hydrogen;
R⁶ is hydrogen or (C₁₋₆)alkyl; or together with R² forms Y;
R⁷ is hydrogen; halogen; hydroxy optionally substituted with (C₁₋₆)alkyl; or (C₁₋₆)alkyl;
Y is CR⁴R⁸CH₂; CH₂CR⁴R⁸; (C═O); CR⁴R⁸; CR⁴R⁸(C═O); or (C═O)CR⁴R⁸;
or when X is CR⁴R⁸, R⁸ and R⁷ together represent a bond;
U is selected from CO and CH₂ and
R⁵ is an optionally substituted bicyclic carbocyclic or heterocyclic ring system (B): 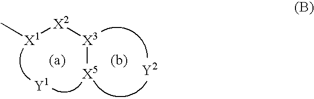
containing up to four heteroatoms in each ring in which
at least one of rings (a) and (b) is aromatic;
X¹ is C or N when part of an aromatic ring, or CR¹⁴ when part of a non-aromatic ring;
X² is N, NR¹³, O, S(O)ₓ, CO or CR¹⁴ when part of an aromatic or non-aromatic ring or may in addition be CR¹⁴R¹⁵ when part of a non aromatic ring;
X³ and X⁵ are independently N or C;
Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring;
Y² is a 2 to 6 atom linker group, each atom of Y² being independently selected from N, NR¹³, O, S(O)ₓ, CO, CR¹⁴ when part of an aromatic or non-aromatic ring or may additionally be CR¹⁴R¹⁵ when part of a non aromatic ring;
each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or
R¹⁴ and R¹⁵ may together represent oxo;
each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; and
each x is independently 0, 1 or 2; and
R⁹ is hydrogen or hydroxy.

2. A compound according to claim 1 wherein Z¹ and Z² together are CH═CH or S.

3. A compound according to claim 1 wherein Z¹ and Z² together are CH═CH, R^{1a} is hydrogen, fluoro or methoxy and R^{1b} is hydrogen.

4. A compound according to claim 1 wherein Z¹ and Z² together are S, R^{1a} is hydrogen, fluoro or chloro and R^{1b} is hydrogen.

5. A compound according to claim 1 wherein Z¹ and Z² together represent CH₂O, R^{1a} is fluoro and R^{1b} is hydrogen.

6. A compound according to claim 1 wherein A is (ia), n is 1 and R³ is H or hydroxy in the 3-position, A is (ii), X is CR⁴R⁸ and R⁸ is H and R⁴ is H or OH, or A is (ii), X is O, R⁷ is H and W¹, W² and W³ are each CH₂.

7. A compound according to claim 1 wherein U is CH₂.

8. A compound according to claim 1 wherein R⁵ is an aromatic heterocyclic ring (B) having 8-11 ring atoms including 2-4 heteroatoms of which at least one is N or NR¹³ in which Y² contains 2-3 heteroatoms, one of which is 5 and 1-2 are N, with one N bonded to X³, or the heterocyclic ring (B) has ring (a) aromatic selected from optionally substituted benzo, pyrido, pyridazino and pyrimidino and ring (b) non aromatic and Y² has 3-5 atoms, including at least one heteroatom, with O, S, CH₂ or NR¹³ bonded to X⁵ where R¹³ is other than hydrogen, and either NHCO bonded via N to X³, or O, S, CH₂ or NH bonded to X³.

9. A compound according to claim 1 wherein R⁵ is selected from the group consisting of:
3-oxo-3,4-dihydro-2H-pyrido[3,2-b][1,4]oxazin-6-yl;
3-oxo-3,4-dihydro-2H-pyrido[3,2-b][1,4]thiazin-6-yl;
6,7-dihydro[1,4]dioxino[2,3-c]pyridazin-3-yl;
6,7-dihydro[1,4]oxathiino[2,3-c]pyridazin-3-yl;
2,3-dihydro-[1,4]dioxino[2,3-c]pyridin-7-yl; and
[1,3]oxathiolo[5,4-c]pyridin-6-yl.

10. A compound according to claim 1 selected from the group consisting of:
1-({4-[(2,3-dihydro[1,4]dioxino[2,3-c]pyridin-7-ylmethyl)amino]-1-piperidinyl}methyl)-2,3-dihydro-1H,5H-pyrido[3,2,1-ij]quinolin-5-one;
1-({4-[(2,3-dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one;
1-({4-[(2,3-dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one (Enantiomer 1);
1-({4-[(2,3-dihydro[1,4]dioxino[2,3-c]pyridin-7-ylmethyl)amino]-1-piperidinyl}methyl)-10-fluoro-2,3-dihydro-1H,5H-pyrido[3,2,1-ij]quinolin-5-one (Enantiomer 2);
1-({4-[(2,3-dihydro[1,4]dioxino[2,3-c]pyridin-7-ylmethyl)amino]-1-piperidinyl}methyl)-8-fluoro-2,3-dihydro-1H,5H-pyrido[3,2,1-ij]quinolin-5-one;
1-({4-[(2,3-dihydro[1,4]dioxino[2,3-c]pyridin-7-ylmethyl)amino]-1-piperidinyl}methyl)-8,10-difluoro-2,3-dihydro-1H,5H-pyrido[3,2,1-ij]quinolin-5-one;
1-({4-[(6,7-dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one (Enantiomer 1);
1-({4-[(6,7-dihydro[1,4]dioxino[2,3-c]pyridazin-3-ylmethyl)amino]-1-piperidinyl}methyl)-10-fluoro-2,3-dihydro-1H,5H-pyrido[3,2,1-ij]quinolin-5-one (Enantiomer 2);
1-({4-[(6,7-dihydro[1,4]oxathiino[2,3-c]pyridazin-3-ylmethyl)amino]-1-piperidinyl}methyl)-10-fluoro-2,3-dihydro-1H,5H-pyrido[3,2,1-ij]quinolin-5-one;
10-fluoro-1-[(4-{[(3-oxo-3,4-dihydro-2H-pyrido[3,2-b][1,4]thiazin-6-yl)methyl]amino}-1-piperidinyl)methyl]-2,3-dihydro-1H,5H-pyrido[3,2,1-ij]quinolin-5-one;
10-fluoro-1-[(4-{[(3-oxo-3,4-dihydro-2H-pyrido[3,2-b][1,4]oxazin-6-yl)methyl]amino}-1-piperidinyl)methyl]-2,3-dihydro-1H,5H-pyrido[3,2,1-ij]quinolin-5-one;
1-({4-[(2,3-dihydro[1,4]dioxino[2,3-c]pyridin-7-ylmethyl)amino]-1-piperidinyl}methyl)-10-(methyloxy)-2,3-dihydro-1H,5H-pyrido[3,2,1-ij]quinolin-5-one;
1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one (diastereomer 1);
1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one (diastereomer 1);
1-({(3R,4S)-4-[(2,3-dihydro[1,4]dioxino[2,3-c]pyridin-7-ylmethyl)amino]-3-hydroxy-1-piperidinyl}methyl)-10-fluoro-2,3-dihydro-1H,5H-pyrido[3,2,1-ij]quinolin-5-one (diastereomer 2);
1-({(3R,4S)-4-[(2,3-dihydro[1,4]dioxino[2,3-c]pyridin-7-ylmethyl)amino]-3-hydroxy-1-piperidinyl}methyl)-8-fluoro-2,3-dihydro-1H,5H-pyrido[3,2,1-ij]quinolin-5-one;
10-fluoro-1-[((3R,4S)-3-hydroxy-4-{[(3-oxo-3,4-dihydro-2H-pyrido[3,2-b][1,4]oxazin-6-yl)methyl]amino}-1-piperidinyl)methyl]-2,3-dihydro-1H,5H-pyrido[3,2,1-ij]quinolin-5-one;
10-fluoro-1-[((3R,4S)-3-hydroxy-4-{[(3-oxo-3,4-dihydro-2H-pyrido[3,2-b][1,4]thiazin-6-yl)methyl]amino}-1-piperidinyl)methyl]-2,3-dihydro-1H,5H-pyrido[3,2,1-ij]quinolin-5-one;
1-({(3R,4S)-4-[(6,7-dihydro[1,4]dioxino[2,3-c]pyridazin-3-ylmethyl)amino]-3-hydroxy-1-piperidinyl}methyl)-10-fluoro-2,3-dihydro-1H,5H-pyrido[3,2,1-ij]quinolin-5-one;
1-({(3R,4S)-4-[(6,7-dihydro[1,4]oxathiino[2,3-c]pyridazin-3-ylmethyl)amino]-3-hydroxy-1-piperidinyl}methyl)-10-fluoro-2,3-dihydro-1H,5H-pyrido[3,2,1-ij]quinolin-5-one;
10-fluoro-1-({(3R,4S)-3-hydroxy-4-[([1,3]oxathiolo[5,4-c]pyridin-6-ylmethyl)amino]-1-piperidinyl}methyl)-2,3-dihydro-1H,5H-pyrido[3,2,1-ij]quinolin-5-one;
1-({4-[(2,3-dihydro[1,4]dioxino[2,3-c]pyridin-7-ylmethyl)amino]-1-piperidinyl}methyl)-10-fluoro-1-hydroxy-2,3-dihydro-1H,5H-pyrido[3,2,1-ij]quinolin-5-one;
1-({4-[(2,3-dihydro[1,4]dioxino[2,3-c]pyridin-7-ylmethyl)amino]-1-piperidinyl}methyl)-8-fluoro-1-hydroxy-2,3-dihydro-1H,5H-pyrido[3,2,1-ij]quinolin-5-one;
1-[(2-{[(2,3-Dihydro[1,4]dioxino[2,3-c]pyridin-7-ylmethyl)amino]methyl}-4-morpholinyl)methyl]-10-fluoro-2,3-dihydro-1H,5H-pyrido[3,2,1-ij]quinolin-5-one;
7-Chloro-6-({4-[(2,3-dihydro[1,4]dioxino[2,3-c]pyridin-7-ylmethyl)amino]-1-piperidinyl}methyl)-5,6-dihydro-4H-[1,3]thiazolo[5,4,3-ij]quinolin-2-one;
7-Chloro-6-({4-[(6,7-dihydro[1,4]dioxino[2,3-c]pyridazin-3-ylmethyl)amino]-1-piperidinyl}methyl)-5,6-dihydro-4H-[1,3]thiazolo[5,4,3-ij]quinolin-2-one;
6-({4-[(2,3-Dihydro[1,4]dioxino[2,3-c]pyridin-7-ylmethyl)amino]-1-piperidinyl}methyl)-5,6-dihydro-4H-[1,3]thiazolo[5,4,3-ij]quinolin-2-one;
6-({4-[(2,3-Dihydro[1,4]dioxino[2,3-c]pyridin-7-ylmethyl)amino]-1-piperidinyl}methyl)-7-fluoro-5,6-dihydro-4H-[1,3]thiazolo[5,4,3-ij]quinolin-2-one;
6-({4-[(2,3-Dihydro[1,4]dioxino[2,3-c]pyridin-7-ylmethyl)amino]-1-piperidinyl}methyl)-7-fluoro-5,6-dihydro-4H-[1,3]thiazolo[5,4,3-ij]quinolin-2-one (Enantiomer 1);
6-({4-[(2,3-Dihydro[1,4]dioxino[2,3-c]pyridin-7-ylmethyl)amino]-1-piperidinyl}methyl)-7-fluoro-5,6-dihydro-4H-[1,3]thiazolo[5,4,3-ij]quinolin-2-one (Enantiomer 2);
6-({4-[(6,7-Dihydro[1,4]dioxino[2,3-c]pyridazin-3-ylmethyl)amino]-1-piperidinyl}methyl)-7-fluoro-5,6-dihydro-4H-[1,3]thiazolo[5,4,3-ij]quinolin-2-one;
7-Fluoro-6-[(4-{[(3-oxo-3,4-dihydro-2H-pyrido[3,2-b][1,4]oxazin-6-yl)methyl]amino}-1-piperidinyl)methyl]-5,6-dihydro-4H-[1,3]thiazolo[5,4,3-ij]quinolin-2-one;
7-Fluoro-6-[(4-{[(3-oxo-3,4-dihydro-2H-pyrido[3,2-b][1,4]thiazin-6-yl)methyl]amino}-1-piperidinyl)methyl]-5,6-dihydro-4H-[1,3]thiazolo[5,4,3-ij]quinolin-2-one;
6-({(3R,4S)-4-[(2,3-dihydro[1,4]dioxino[2,3-c]pyridin-7-ylmethyl)amino]-3-hydroxy-1-piperidinyl}methyl)-7-fluoro-5,6-dihydro-4H-[1,3]thiazolo[5,4,3-ij]quinolin-2-one;
7-({4-[(2,3-Dihydro[1,4]dioxino[2,3-c]pyridin-7-ylmethyl)amino]-1-piperidinyl}methyl)-8-fluoro-6,7-dihydro-5H-[1,4]oxazino[2,3,4-ij]quinolin-3(2H)-one;
7-({4-[(6,7-Dihydro [1,4]dioxino[2,3-c]pyridazin-3-ylmethyl)amino]-1-piperidinyl}methyl)-8-fluoro-6,7-dihydro-5H-[1,4]oxazino[2,3,4-ij]quinolin-3(2H)-one;
8-Fluoro-7-[(4-{[(3-oxo-3,4-dihydro-2H-pyrido[3,2-b][1,4]oxazin-6-yl)methyl]amino}-1-piperidinyl)methyl]-6,7-dihydro-5H-[1,4]oxazino[2,3,4-ij]quinolin-3(2H)-one;
8-Fluoro-7-{[(3S,4S)-3-hydroxy-4-({[(3-oxo-3,4-dihydro-2H-pyrido[3,2-b][1,4]oxazin-6-yl)methyl]amino}methyl)-1-pyrrolidinyl]methyl}-6,7-dihydro-5H-[1,4]oxazino[2,3,4-ij]quinolin-3(2H)-one; and
8-Fluoro-7-{[(3S,4S)-3-hydroxy-4-({[(3-oxo-3,4-dihydro-2H-pyrido[3,2-b][1,4]thiazin-6-yl)methyl]amino}methyl)-1-pyrrolidinyl]methyl}-6,7-dihydro-5H-[1,4]oxazino[2,3,4-ij]quinolin-3(2H)-one;
or a pharmaceutically acceptable salt thereof.

11. A method of treatment of bacterial infections in mammals, particularly in man, which method comprises the administration to a mammal in need of such treatment an effective amount of a compound according to claim 1.

12. (canceled)
